SEQUENCE LISTING (11 sequences)

SEQ ID NO: 1 - DNA, 20 bp, Artificial Sequence
  primer
aacacatttcaagccccaaa

SEQ ID NO: 2 - DNA, 24 bp, Artificial Sequence
  primer
gaaactggtttcaaaatattcgtt

SEQ ID NO: 3 - DNA, 23 bp, Artificial Sequence
  primer
gctttttctgtaaatcatctgtg

SEQ ID NO: 4 - DNA, 21 bp, Artificial Sequence
  primer
ctgagatcagccaaattcagt

SEQ ID NO: 5 - DNA, 20 bp, Artificial Sequence
  primer
catttgctccaaactgacca

SEQ ID NO: 6 - DNA, 20 bp, Artificial Sequence
  primer
tactccaaagcctcttgctc

SEQ ID NO: 7 - DNA, 20 bp, Artificial Sequence
  primer
acattcgaaagaccctagcc

SEQ ID NO: 8 - DNA, 21 bp, Artificial Sequence
  primer
caattcctatgcaatcggtct

SEQ ID NO: 9 - protein, 188 aa, Homo sapiens [sequence omitted]

SEQ ID NO: 10 - protein, 766 aa, Homo sapiens [sequence omitted]

SEQ ID NO: 11 - protein, 1068 aa, Homo sapiens
MPPRPSSGELWGIHLMPPRILVECLLPNGMIVTLECLREATLITIKHELFKEARKYPLHQLLQDESSYIFVSVTQEAEREEFFDETRRLCDLRLFQPFLKVIEPVGNREEKILNREIGFAIGMPVCEFDMVKDPEVQDFRRNILNVCKEAVDLRDLNSPHSRAMYVYPPNVESSPELPKHIYNKLDKGQIIVVIWVIVSPNNDKQKYTLKINHDCVPEQVIAEAIRKKTRSMLLSSEQLKLCVLEYQGKYILKVCGCDEYFLEKYPLSQYKYIRSCIMLGRMPNLMLMAKESLYSQLPMDCFTMPSYSRRISTATPYMNGETSTKSLWVINSALRIKILCATYVNVNIRDIDKIYVRTGIYHGGEPLCDNVNTQRVPCSNPRWNEWLNYDIYIPDLPRAARLCLSICSVKGRKGAKEEHCPLAWGNINLFDYTDTLVSGKMALNLWPVPHGLEDLLNPIGVTGSNPNKETPCLELEFDWFSSVVKFPDMSVIEEHANWSVSREAGFSYSHAGLSNRLARDNELRENDKEQLKAISTRDPLSEITEQEKDFLWSHRHYCVTIPEILPKLLLSVKWNSRDEVAQMYCLVKDWPPIKPEQAMELLDCNYPDPMVRGFAVRCLEKYLTDDKLSQYLIQLVQVLKYEQYLDNLLVRFLLKKALTNQRIGHFFFWHLKSEMHNKTVSQRFGLLLESYCRACGMYLKHLNRQVEAMEKLINLTDILKQEKKDETQKVQMKFLVEQMRRPDFMDALQGFLSPLNPAHQLGNLRLEECRIMSSAKRPLWLNWENPDIMSELLFQNNEIIFKNGDDLRQDMLTLQIIRIMENIWQNQGLDLRMLPYGCLSIGDCVGLIEVVRNSHTIMQIQCKGGLKGALQFNSHTLHQWLKDKNKGEIYDAAIDLFTRSCAGYCVATFILGIGDRHNSNIMVKDDGQLFHIDFGHFLDHKKKKFGYKRERVPFVLTQDFLIVISKGAQECTKTREFERFQEMCYKAYLAIRQHANLFINLFSMMLGSGMPELQSFDDIAYIRKTLALDKTEQEALEYFMKQMNDAHHGGWTTKMDWIFHTIKQHALN

(19) United States
(12) Patent Application Publication
(10) Pub. No.: US 2011/0217309 A1
(43) Pub. Date: Sep. 8, 2011

(54) BIOLOGICAL MARKERS PREDICTIVE OF ANTI-CANCER RESPONSE TO INSULIN-LIKE GROWTH FACTOR-1 RECEPTOR KINASE INHIBITORS

(76) Inventors: Elizabeth A. Buck, Huntington, NY (US); Sharon M. Barr, Huntington, NY (US); Mark R. Miglarese, Superior, CO (US)

(21) Appl. No.: 13/039,717

(22) Filed: Mar. 3, 2011

Related U.S. Application Data
(60) Provisional application No. 61/310,038, filed on Mar. 3, 2010.

(51) Int. Cl. A61K 39/395; C12Q 1/68; A61K 31/4985; A61P 35/00

(52) U.S. Cl. 424/142.1; 435/6.11; 514/249; 424/158.1

(57) ABSTRACT

The present invention provides diagnostic methods for predicting the effectiveness of treatment of an ovarian cancer patient with an IGF-1R kinase inhibitor. Methods are provided for predicting the sensitivity of tumor cell growth to inhibition by an IGF-1R kinase inhibitor, comprising assessing whether the tumor cells possess mutant K-RAS. The present invention thus provides a method of identifying patients with ovarian cancer who are most likely to benefit from treatment with an IGF-1R kinase inhibitor. Improved methods for treating cancer patients with IGF-1R kinase inhibitors that incorporate this methodology are also provided. The present invention also provides diagnostic methods for predicting the effectiveness of treatment of cancer patients with IGF-1R kinase inhibitors, based on a determination of the mutation status of the genes K-RAS, B-RAF, PTEN and PIK3CA, which can be used to identify tumor cell types that will be sensitive to IGF-1R kinase inhibitors, and also those that will be insensitive.

### CROSS-REFERENCE TO RELATED APPLICATIONS

This application claims the benefit of U.S. Provisional Application No. 61/310,038, filed Mar. 3, 2010, which is herein incorporated by reference in its entirety.

### BACKGROUND OF THE INVENTION

Cancer is a generic name for a wide range of cellular malignancies characterized by unregulated growth, lack of differentiation, and the ability to invade local tissues and metastasize. These neoplastic malignancies affect, with various degrees of prevalence, every tissue and organ in the body. The present invention is directed to methods for diagnosing and treating cancer patients. In particular, the present invention is directed to methods for determining which patients will most benefit from treatment with an insulin-like growth factor-1 receptor (IGF-1R) kinase inhibitor.

IGF-1R belongs to the insulin receptor family that includes the Insulin Receptor (IR), IGF-1R (homodimer), IGF-1R/IR (hybrid receptor), and IGF-2R (mannose 6-phosphate receptor). IGF-1R/IR hybrids act as homodimers, preferentially binding and signaling with IGFs. IR exists in two isoforms: IR-B (traditional insulin receptor) and IR-A (a fetal form which is re-expressed in selected tumors and preferentially binds IGF-II). IGF-2R is a non-signaling receptor that acts as a “sink” for IGF-II (Pollak M. N., et al. Nat Rev Cancer 2004 4:505-18). Six well-characterized insulin-like growth factor binding proteins (IGFBP-1 through -6) associate with IGF ligands to stabilize the IGFs and modulate their ability to bind the IGF-IR.

IGF-1R is a transmembrane RTK that binds primarily to IGF-1 but also to IGF-II and insulin with lower affinity. Binding of IGF-1 to its receptor results activation of receptor tyrosine kinase activity, intermolecular receptor autophosphorylation and phosphorylation of cellular substrates (major substrates are IRS1 and Shc). The ligand-activated IGF-1R induces mitogenic activity in normal cells and plays an important role in abnormal growth. A major physiological role of the IGF-1 system is the promotion of normal growth and regeneration. Overexpressed IGF-1R (type 1 insulin-like growth factor receptor) can initiate mitogenesis and promote ligand-dependent neoplastic transformation. Furthermore, IGF-1R plays an important role in the establishment and maintenance of the malignant phenotype. Unlike the epidermal growth factor (EGF) receptor, no mutant oncogenic forms of the IGF-1R have been identified. However, several oncogenes have been demonstrated to affect IGF-1 and IGF-1R expression. The correlation between a reduction of IGF-1R expression and resistance to transformation has been seen. Exposure of cells to the mRNA antisense to IGF-1R RNA prevents soft agar growth of several human tumor cell lines. IGF-1R abrogates progression into apoptosis, both in vivo and in vitro. It has also been shown that a decrease in the level of IGF-1R below wild-type levels causes apoptosis of tumor cells in vivo. The ability of IGF-1R disruption to cause apoptosis appears to be diminished in normal, non-tumorigenic cells.

The IGF-1 pathway has an important role in human tumor development. IGF-1R overexpression is frequently found in various tumors (breast, colon, lung, sarcoma) and is often associated with an aggressive phenotype. High circulating IGF1 concentrations are strongly correlated with prostate, lung and breast cancer risk. Furthermore, IGF-1R is required for establishment and maintenance of the transformed phenotype in vitro and in vivo (Baserga R. Exp. Cell. Res., 1999, 253, 1-6). The kinase activity of IGF-1R is essential for the transforming activity of several oncogenes: EGFR, PDGFR, SV40 T antigen, activated Ras, Raf, and v-Src. The expression of IGF-1R in normal fibroblasts induces neoplastic phenotypes, which can then form tumors in vivo. IGF-1R expression plays an important role in anchorage-independent growth. IGF-1R has also been shown to protect cells from chemotherapy-, radiation-, and cytokine-induced apoptosis. Conversely, inhibition of endogenous IGF-1R by dominant negative IGF-1R, triple helix formation or antisense expression vector has been shown to repress transforming activity in vitro and tumor growth in animal models. The IGF-1R signaling pathway also appears to be a robust target in colorectal cancer (CRC), based upon data demonstrating overexpression of the receptor and ligands in CRC, association with a more malignant phenotype, chemotherapy resistance, and correlation with a poor prognosis (Saltz, L. B., et al. J Clin Oncol 2007; 25(30): 4793-4799; Tripkovic I., et al. Med Res. 2007 July; 38(5):519-25. Epub 2007 Apr. 26; Miyamoto S., et al. Clin Cancer Res. 2005 May 1; 11(9):3494-502; Nakamura M., et al. Clin Cancer Res. 2004 Dec. 15; 10(24):8434-41; Grothey A, et al. J Cancer Res Clin Oncol. 1999; 125(3-4):166-73).

It has been recognized that inhibitors of protein-tyrosine kinases are useful as selective inhibitors of the growth of mammalian cancer cells. For example, Gleevec™ (also known as imatinib mesylate), a 2-phenylpyrimidine tyrosine kinase inhibitor that inhibits the kinase activity of the BCR-ABL fusion gene product, has been approved by the U.S. Food and Drug Administration for the treatment of CML. The 4-anilinoquinazoline compound Tarceva™ (erlotinib HCl) has also been approved by the FDA, and selectively inhibits EGF receptor kinase with high potency. The development for use as anti-tumor agents of compounds that directly inhibit the kinase activity of IGF-1R, as well as antibodies that reduce IGF-1R kinase activity by blocking IGF-1R activation or antisense oligonucleotides that block IGF-1R expression, are areas of intense research effort (e.g. see Larsson, O. et al (2005) Brit. J. Cancer 92:2097-2101; Ibrahim, Y. H. and Yee, D. (2005) Clin. Cancer Res. 11:944s-950s; Mitsiades, C. S. et al. (2004) Cancer Cell 5:221-230; Camirand, A. et al. (2005) Breast Cancer Research 7:R570-R579 (DOI 10.1186/bcr1028); Camirand, A. and Pollak, M. (2004) Brit. J. Cancer 90:1825-1829; Garcia-Echeverria, C. et al. (2004) Cancer Cell 5:231-239; Sachdev D, and Yee D., Mol Cancer Ther. 2007 January; 6(1):1-12; Hofmann F., and Garcia-Echeverria C., Drug Discov Today 2005 10:1041-7). Agents inhibiting the IGF-1R pathway have demonstrated anti-tumor efficacy in multiple human cancer models both in vitro and in vivo, particularly in pediatric models of Ewing's sarcoma and rhabdomyosarcoma (Manara M C, et al. Int J Oncol 2005 27:1605-16). Despite early hints of efficacy in patients with sarcoma, results to date of IGF-1R inhibitors in early clinical trials have not been impressive, indicating that patient selection strategies and rational combinations may be needed to move forward with this approach (Tolcher A. W., et al. Journal of Clinical Oncology, 2007 ASCO Annual Meeting Proceedings Part I. Vol 25, No. 18S (June 20 Supplement), 2007: 3002). Data acquired this far, has not indicated that activation, overexpression, or amplification of members of the IGF-1R pathway will predict responsiveness.

IGF-1R/IR signaling can mediate activation of cellular survival in the presence of a multitude of other anti-tumor agents including cytotoxic chemotherapeutics and radiation as well as molecular targeted therapies (MTTs). The ability for IGF-1R/IR inhibitors to augment the efficacy for these agents has been extensively investigated in the preclinical setting and is currently being actively persued in the clinical setting. Resistance to both radiation and cytotoxic chemotherapies can be associated with increased activity through the AKT survival pathway, which can be driven by IGF-1R/IR signaling. Radiation treatment achieves augmented anti-tumor activity upon co-administration of an IGF-1R antagonist in in vivo xenograft models. In numerous settings IGF-1R inhibitors have been shown to augment the cytotoxic effects for chemotherapies including paclitaxel and doxorubicin (Wang, Y. H. et al., Mol. Cell Biochem., 2009, 327, 257; Allen, G. W. et al. Cancer Res., 2007, 67, 1155; Zeng, X., et al. Clin. Cancer Res., 2009, 15, 2840; Martins, A. S. et al. Clin. Cancer Res., 2006, 12, 3532). Similar to observations with radiation, tumor cells can also upregulate AKT survival signaling in response to cytotoxic chemotherapies. Recent studies have shown that cytotoxic agents including paclitaxel can evoke specific upregulation of IGF-1R activity, and IGF-1R inhibitors can augment the pro-apoptotic potential for such agents (P. Chinnaiyan, G. W. et al., (2006) Semin. Radiat. Oncol., 16, 59-64). These preclinical data have provided strong rationale for a multitude of clinical studies evaluating IGF-1R inhibitors in combination with chemtherapeutics.

Several groups have investigated or disclosed potential biomarkers to predict a patient's response to protein-tyrosine kinase inhibitors (see for example, PCT publications: WO 2004/063709, WO 2005/017493, WO 2004/111273, WO 2008/108986, WO 2007/001868 and WO 2004/071572; and US published patent applications: US 2005/0019785, US 2007/0065858, US 2009/0092596, US 2009/0093488, US 2006/0140960 and US 2004/0132097). Several biomarkers have been proposed for predicting the response to EGFR kinase inhibitors, including mutant KRAS as a predictor of non-responsiveness in colorectal cancer (e.g. see Brugger, W. et al. (2009) J Clin Oncol 27:15s, (suppl; abstr 8020); Siena, S et al (2009) JNCI 101(19):1308-1324; Riely and Ladanyi (2008) J Mol Diagnostics 10(6):493; Jimeno, A. et al. (2009) Cancer J. 15(2):110-13). In addition, several biomarkers, including mutant KRAS, have been disclosed that have potential in predicting a patient's response to IGF-1R kinase inhibitors, (e.g. see Rodon, J. et al (2008) Mol Cancer Ther. 7:2575-2588; T. Pitts et al. (2009) EORTC Conference, Boston, Mass., abstract #2141; Huang, F. et al. (2009) Cancer Res. 69(1):161-170; Rodon, J. et al., (2008) Mol. Cancer Ther. 7:2575-2588). However, in most instances no FDA-approved diagnostic tests have yet emerged that can effectively guide practicing physicians in the treatment of their patients with such inhibitors, or can indicate to the physician which tumors will respond most favorable to a combination of such an inhibitor with a standard chemotherapy agent.

The human KRAS gene is mutated in over 30% of colorectal cancers, and in many other tumor types. Somatic missense mutations in the KRAS gene lead to single amino acid substitutions. The most frequent alterations are detected in codons 12 and 13 in exon 2 of the KRAS gene. Mutations in other positions, such as codons 61 and 146, have also been reported, but these alterations account for a minor proportion of KRAS mutations. KRAS mutations in codons 12 and 13 appear to play a major role in the progression of colorectal cancer. The KRAS gene encodes a small G-protein that functions downstream in many receptor signaling pathways (e.g. EGFR, IGF-1R). It belongs to the family of RAS proteins that are involved in coupling signal transduction from cell surface receptors to intracellular targets via several signaling cascades, including the RAS-MAPK pathway. RAS proteins normally cycle between active GTP-bound (RAS-GTP) and inactive GDP-bound (RAS-GDP) conformations. RAS proteins are activated by guanine nucleotide exchange factors (GEFs), which are recruited to protein complexes at the intracellular domain of activated receptors. Signaling is terminated when RAS-GTP is hydrolyzed to the RAS-GDP inactive complex by GTPase-activating proteins (GAPs). Under physiological conditions, levels of RAS-GTP in vivo are tightly controlled by the counterbalancing activities of GEFs and GAPs. Mutations in genes that encode RAS proteins disrupt this balance, causing perturbations in downstream signaling activities. KRAS mutations result in RAS proteins that are permanently in the active GTP-bound form due to defective intrinsic GTPase activity and resistance to GAPs. Unlike wild-type RAS proteins which are inactivated after a short time, the aberrant proteins are able to continuously activate signaling pathways in the absence of any upstream stimulation of protein-tyrosine kinase receptors. Oncogenic activation of RAS signaling pathways has been implicated in many aspects of the malignant process, including abnormal cell growth, proliferation, and differentiation. KRAS mutations are, in most cases, an early event in the development and progression of colorectal cancers. Consistent with this concept, several studies have demonstrated that KRAS mutation status is an important prognostic factor in colorectal cancer.

The human B-RAF gene encodes a protein belonging to the raf/mil family of serine/threonine protein kinases. This protein plays a role in regulating the MAP kinase/ERKs signaling pathway, which affects cell division, differentiation, and secretion. Activating mutations of the B-RAF gene play a central role in the development of various cancer types, including non-Hodgkin lymphoma, colorectal cancer, malignant melanoma, papillary thyroid carcinoma, non-small cell lung carcinoma, and adenocarcinoma of lung. Over 30 single site missense mutations have been identified in human B-RAF, mostly located within the kinase domain. Significantly, one activating mutation, a glutamate (E) for valine (V) substitution at residue 600 in the activation segment, accounts for 90% of B-RAF mutations in human cancers. This V600E mutant has greatly elevated kinase activity, and constitutively stimulates the MAP kinase pathway in vivo, independent of RAS.

Phosphatidylinositol-3-kinases (PI 3-kinases or PI3Ks) are a family of enzymes involved in cellular functions such as cell growth, proliferation, differentiation, motility, survival and intracellular trafficking. Class I PI3Ks are responsible for the production of phosphatidylinositol 3-phosphate, are composed of a catalytic subunit known as p110 and a regulatory subunit p85, and are activated by G-protein coupled receptors and tyrosine kinase receptors. One of the human PI3K catalytic subunits is expressed by the gene PIK3CA, which is mutated in a number of human cancers. Somatic missense mutations cluster in specific domains, similar to that observed for activating mutations in other oncogenes, such as K-RAS and B-RAF. Mutant PIK3CA has increased lipid kinase activity compared to the wild-type protein. The most common activating mutations in PIK3CA are E542K, E545K, and H1047R.

The product of the tumor suppressor gene PTEN (Phosphatase and tensin homologue, also known as MMAC or PTEN-1) is a dual specificity phosphatase and has been shown to dephosphorylate inositol phospholipids in vivo, and has an important role in controlling cell growth, inducing cell cycle arrest, promoting apoptosis, down regulating adhesion and suppressing cell migration. The PTEN gene, which is located on the short arm of chromosome 10 (10q23), is mutated and/or deleted in 40-50% of high grade gliomas as well as many other tumor types, including those of the prostate, brain, endometrium, thyroid, breast, and lung, and a role for epigenetic and genetic changes of PTEN has been demonstrated in the development of sequamous cell carcinoma (SCC) of the cervix. In addition, PTEN is mutated in several rare autosomal dominant cancer predisposition syndromes, including Cowden disease, Lhermitte-Duclos disease and Bannayan-Zonana syndrome.

There remains a critical need for improved methods for determining the best mode of treatment for any given cancer patient. The present invention provides methods for determining which tumors will respond most effectively to treatment with IGF-1R kinase inhibitors based on whether the tumor cells possess mutant KRAS, B-RAF, PTEN and PIK3CA biomarkers, and for the incorporation of such a determination into more effective treatment regimens for cancer patients with IGF-1R kinase inhibitors.

### SUMMARY OF THE INVENTION

The present invention provides new diagnostic methods using mutant gene biomarkers for predicting the effectiveness of treatment of cancer patients with IGF-1R kinase inhibitors, and improved methods for treating cancer patients with IGF-1R kinase inhibitors that utilize said diagnostic methods prior to administration of drug.

The present invention provides diagnostic methods for predicting the effectiveness of treatment of an ovarian cancer patient with an IGF-1R kinase inhibitor. These methods are based on the surprising discovery that the sensitivity of ovarian tumor cell growth to inhibition by IGF-1R kinase inhibitors is predicted by whether such tumor cells possess a mutant K-RAS gene, wherein tumor cells that possess the latter are more sensitive to inhibition than tumor cells that possess wild type K-RAS.

Improved methods for treating ovarian cancer patients with IGF-1R kinase inhibitors that incorporate the above methodology are also provided. Thus, the present invention further provides a method for treating ovarian tumors or tumor metastases in a patient, comprising the steps of diagnosing a patient's likely responsiveness to an IGF-1R kinase inhibitor by assessing whether the tumor cells possess a mutant K-RAS gene, and administering to said patient a therapeutically effective amount of an IGF-1R kinase inhibitor (e.g. OSI-906) if the tumor cells possess mutant K-RAS.

The present invention also provides diagnostic methods for predicting the effectiveness of treatment of cancer patients with IGF-1R kinase inhibitors, based on a determination of the mutation status of the genes K-RAS, B-RAF and PIK3CA, which can be used to identify tumor cell types that will be sensitive to IGF-1R kinase inhibitors, and also many of those that will be insensitive.

For example, the invention provides a method of identifying patients with cancer who are most likely to benefit or not benefit from treatment with an IGF-1R kinase inhibitor, comprising: obtaining a sample of a patient's tumor, determining if tumor cells of the sample possess a mutant K-RAS gene; determining if tumor cells of the sample possess a mutant B-RAF gene; determining if tumor cells of the sample possess a mutant PIK3CA gene; and identifying the patient as likely to benefit from treatment with an IGF-1R kinase inhibitor if mutant K-ras or mutant B-RAF is present in the tumor cells of the patient in the absence of mutant PIK3CA expression; and identifying the patient as likely to not benefit from treatment with an IGF-1R kinase inhibitor if mutant PIK3CA is present in the tumor cells of the patient. The invention also provides methods of identifying patients with cancer who are not likely to benefit from treatment with an IGF-1R kinase inhibitor, based on a determination of the presence of mutant PIK3CA or PTEN expression in their tumor cells, which correlates with a relative lack of sensitivity of these cells to IGF-1R kinase inhibitors.

Improved methods for treating cancer patients with IGF-1R kinase inhibitors that incorporate the above methods are also provided. Thus, the invention also provides a method for treating cancer in a patient, comprising the steps of: (A) diagnosing a patient's likely responsiveness to an IGF-1R kinase inhibitor by determining if the patient has a tumor that is likely to respond to treatment with an IGF-1R kinase inhibitor by: obtaining a sample of the patient's tumor, determining if tumor cells of the sample possess a mutant K-RAS gene; determining if tumor cells of the sample possess a mutant B-RAF gene; determining if tumor cells of the sample possess a mutant PIK3CA gene; and identifying the patient as likely to benefit from treatment with an IGF-1R kinase inhibitor if mutant K-ras or mutant B-RAF is present in the tumor cells of the patient in the absence of mutant PIK3CA; and identifying the patient as likely to not benefit from treatment with an IGF-1R kinase inhibitor if mutant PIK3CA is present in the tumor cells of the patient; and (B) administering to said patient a therapeutically effective amount of an IGF-1R kinase inhibitor (e.g. OSI-906) if the patient is diagnosed to be potentially responsive to an IGF-1R kinase inhibitor. The invention also provides a method for treating cancer in a patient, comprising administering to said patient a therapeutically effective amount of an IGF-1R kinase inhibitor if the patient has been diagnosed to be potentially responsive to an IGF-1R kinase inhibitor by a determination that the tumor cells of the patient do not possess a mutant PTEN gene or a mutant PIK3CA gene.

### BRIEF DESCRIPTION OF THE FIGURES

FIG. 1: KRAS mutation status correlates with OSI-906 sensitivity for OvCa tumor cell lines. The effect of varying concentrations of OSI-906 (IGF-1R TKI) on cell proliferation for a panel of eight ovarian carcinoma (OvCa) tumor cell lines. Proliferation was assayed using Cell Titer Glo (Promega) and was determined 72 hours following dosing with OSI-906. KRAS mutation status, as reported by the Sanger Wellcome Trust, is noted. Results shown are typical of three or more independent experiments. Grey symbols indicate data for K-RAS mutant (mt) cell lines OVCAR-5 and MDAH2774. Black symbols indicate data for K-RAS wild-type (wt) cell lines OVCAR-4, SKOV-3, OVCAR-3, OVCAR-8, CaOV3-5, and IGROV-1.

FIG. 2: KRAS mutation status and OSI-906 sensitivity correlates with elevated expression of IGF2 ligand. The activation states for IGF-1R and IR and IGF2 transcript expression were determined for the OSI-906 sensitive tumor cell line MDAH-2774 and the OSI-906 insensitive cell lines OVK18 and OVCAR4. pIGF-1R and pIR were determined by RTK capture array (RTK Proteome Profiler, R&D Systems), and the expression of IGF2 mRNA was determined by quantitative PCR. Results shown are typical of three or more independent experiments. The open arrow indicates cell line data for the K-RAS mutant (mt) cell line MDAH2774. The other two cell lines, OVK18 and OVCAR4 (closed arrows), have wild type KRAS.

FIG. 3: Synergism for OSI-906 in combination with paclitaxel can be predicted by KRAS mutation status. The effect of OSI-906 in combination with paclitaxel was determined for the panel of eight OvCa tumor cell lines. Synergy is expressed as the fold gain in maximal efficacy in excess of that predicted for additivity as assessed using the BLISS drug combination effect model. IGF2 transcript expression, as determined by quantitative PCR is shown, and the KRAS mutation status for each cell line is indicated. Results are typical of three or more independent experiments. Open arrows indicate cell line data for K-RAS mutant (mt) cell lines OVCAR-5 and MDAH2774. All other cell lines (closed arrows) have wild type KRAS.

FIG. 4: The IGF-1R kinase inhibitor OSI-906 in combination with paclitaxel synergistically inhibits ovarian tumor cell growth. A. Effect of 3 nM or 10 nM paclitaxel in combination with OSI-906 on MDAH-2774 ovarian tumor cell growth. The dotted line in the plot represents the calculated theoretical expectation if the combination was additive in nature, and was determined using the Bliss model for additivity. B. Effect of OSI-906 on the induction of apoptosis by 10 nM pactitaxel in MDAH-2774 ovarian tumor cells. C. Effect of 5 μmM OSI-906 on the phosphorylation of Akt at varying concentrations of pactitaxel (left to right, 100, 30, 10, 3, and 1 nM).

FIG. 5: Expression in tumor cells of either mutant K-RAS or mutant B-RAF, in the absence of mutant PIK3CA, is predictive of sensitivity of tumor cell growth to IGF-1R kinase inhibitors. A. Sensitivity to OSI-906 for a panel of 32 tumor cell lines derived from 10 tumor types, expressed as EC₅₀ values. Cell lines were categorized as either sensitive (EC₅₀<1 μM) or insensitive (EC₅₀>10 μM) to OSI-906. Mutational status for KRAS, BRAF, and PIK3CA is indicated, as reported by the Sanger Wellcome database (Sanger Wellcome Trust, Wellcome Trust Genome Campus, Hinxton, Cambridge, CB10 1SA, UK; internet address—www.sanger.ac.uk/genetics/CGP/cosmic/), or other literature sources described herein below. Those mutation statuses that are not reported are shaded grey. B. Effect of varying concentrations of OSI-906 on cell growth for a representative panel of 5 sensitive tumor cell lines.

FIG. 6: Protein sequence of c-K-ras2 protein isoform b precursor [Homo sapiens], NCBI Reference Sequence: NP_{—}004976.2, encoded by the human K-RAS gene (GeneID: 3845). Amino acid residues encoded by codons 12, 13 and 61 are underlined.

FIG. 7: Protein sequence of B-Raf [Homo sapiens], NCBI Reference Sequence: NP_{—}004324.2, encoded by the human B-RAF gene (GeneID: 673). Amino acid residues encoded by codons 600 and 601 are underlined.

FIG. 8: Protein sequence of phosphoinositide-3-kinase, catalytic, alpha polypeptide [Homo sapiens], NCBI Reference Sequence: NP_{—}006209.2, encoded by the human PIK3CA gene (GeneID: 5290). Amino acid residues encoded by codons 111, 542, 545, 549, and 1047 are underlined.

FIG. 9: Expression in tumor cells of either mutant K-RAS or mutant B-RAF, in the absence of mutant PIK3CA, is predictive of sensitivity of tumor cell growth to IGF-1R kinase inhibitors, and expression in tumor cells of mutant PTEN or PI3K is predictive of insensitivity of tumor cell growth to IGF-1R kinase inhibitors. Sensitivity to OSI-906 for a panel of 50 tumor cell lines derived from 12 tumor types, including NSCLC, CRC, breast, ovarian cancer, hepatocellular carcinoma, multiple myeloma and Ewings sarcoma, expressed as EC₅₀ values. Cell lines were categorized as either sensitive (EC₅₀<1 μM) or insensitive (EC₅₀>10 μM) to OSI-906. Mutational status for KRAS, BRAF, PTEN and PIK3CA is indicated, as reported by the Sanger Wellcome database (Sanger Wellcome Trust, Wellcome Trust Genome Campus, Hinxton, Cambridge, CB10 1SA, UK; internet address www.sanger.ac.uk/genetics/CGP/cosmic/), or other literature sources described herein below. Those mutation statuses that are not reported are shaded grey.

### DETAILED DESCRIPTION OF THE INVENTION

The term “cancer” in a patient refers to the presence of cells possessing characteristics typical of cancer-causing cells, such as uncontrolled proliferation, immortality, metastatic potential, rapid growth and proliferation rate, and certain characteristic morphological features. Often, cancer cells will be in the form of a tumor, but such cells may exist alone within the subject, or may circulate in the blood stream as independent cells, such as leukemic cells.

“Cell growth”, as used herein, for example in the context of “tumor cell growth”, unless otherwise indicated, is used as commonly used in oncology, where the term is principally associated with growth in cell numbers, which occurs by means of cell reproduction (i.e. proliferation) when the rate of the latter is greater than the rate of cell death (e.g. by apoptosis or necrosis), to produce an increase in the size of a population of cells, although a small component of that growth may in certain circumstances be due also to an increase in cell size or cytoplasmic volume of individual cells. An agent that inhibits cell growth can thus do so by either inhibiting proliferation or stimulating cell death, or both, such that the equilibrium between these two opposing processes is altered.

“Tumor growth” or “tumor metastases growth”, as used herein, unless otherwise indicated, is used as commonly used in oncology, where the term is principally associated with an increased mass or volume of the tumor or tumor metastases, primarily as a result of tumor cell growth.

“Abnormal cell growth”, as used herein, unless otherwise indicated, refers to cell growth that is independent of normal regulatory mechanisms (e.g., loss of contact inhibition). This includes, for example, the abnormal growth of: (1) tumor cells (tumors) that proliferate by expressing a mutated tyrosine kinase or overexpression of a receptor tyrosine kinase; (2) benign and malignant cells of other proliferative diseases in which aberrant tyrosine kinase activation occurs; (3) any tumors that proliferate by receptor tyrosine kinases; (4 any tumors that proliferate by aberrant serine/threonine kinase activation; and (5) benign and malignant cells of other proliferative diseases in which aberrant serine/threonine kinase activation occurs.

The term “treating” as used herein, unless otherwise indicated, means to give medical aid to counteract a disease or condition. The phrase “a method of treating” or its equivalent, when applied to cancer refers to a procedure or course of action that is designed to reduce or eliminate the number of cancer cells in a patient, or to alleviate the symptoms of a cancer. “A method of treating” cancer or another proliferative disorder does not necessarily mean that the cancer cells or other disorder will, in fact, be eliminated, that the number of cells or disorder will, in fact, be reduced, or that the symptoms of a cancer or other disorder will, in fact, be alleviated. Often, a method of treating cancer will be performed even with a low likelihood of success, but which, given the medical history and estimated survival expectancy of a patient, is nevertheless deemed an overall beneficial course of action.

The term “therapeutically effective agent” means a composition that will elicit the biological or medical response of a tissue, system, or human that is being sought by the researcher, medical doctor or other clinician.

The term “therapeutically effective amount” or “effective amount” means the amount of the subject compound or combination that will elicit the biological or medical response of a tissue, system, or human that is being sought by the researcher, medical doctor or other clinician.

The terms “responsive”or “responsiveness” when used herein in referring to a patient's reaction to administration of an IGF-1R kinase inhibitor that inhibits both IGF-1R and IR kinases, refer to a response that is positive or effective, from which the patient is likely to benefit.

The NCBI GeneID numbers listed herein are unique identifiers of genes from the NCBI Entrez Gene database record (National Center for Biotechnology Information (NCBI), U.S. National Library of Medicine, 8600 Rockville Pike, Building 38A, Bethesda, Md. 20894; Internet address www.ncbi.nlm.nih.gov/).

The data presented in the Experimental Details section herein below demonstrates that ovarian tumor cells show a range of sensitivities to growth inhibition by an IGF-1R kinase inhibitor (e.g. OSI-906) and that the degree of sensitivity of the tumor cells to an IGF-1R kinase inhibitor can be assessed by determining the presence or absence of mutant K-RAS in the tumor cells, such that the presence of mutant K-RAS is indicative that the cells are likely to have high sensitivity to growth inhibition by an IGF-1R kinase inhibitor, or conversely, the absence of mutant K-RAS (i.e. wild type K-RAS) is indicative that the cells are likely to have low sensitivity, or be relatively resistant, to growth inhibition by an IGF-1R kinase inhibitor. Thus, these observations can form the basis of valuable new diagnostic methods for predicting the effects of IGF-1R kinase inhibitors on ovarian tumor growth, and give oncologists an additional biomarker to assist them in choosing the most appropriate treatment for their patients.

The data presented in the Experimental Details section herein below also demonstrates that in tumor cell types other than ovarian, K-RAS or B-RAF mutations are found in tumor cells that are sensitive as well as those that are resistant to IGF-1R inhibitors, though such mutations occurred more frequently in IGF-1R kinase inhibitor-sensitive tumor cell lines. In contrast, mutations in PIK3CA were observed in about half of the IGF-1R kinase inhibitor-insensitive tumor cell lines for which the mutational status is known, but occurred in few cell lines that were sensitive to an IGF-1R kinase inhibitor, and can thus be used as a biomarker for insensitivity to IGF-1R kinase inhibitors (e.g. OSI-906). Similarly, mutations in PTEN were also associated with lack of tumor cell sensitivity to IGF-1R kinase inhibitors (e.g. OSI-906), and have not been found in sensitive tumor cells. Furthermore, the data indicates that the presence in tumor cells of either mutant K-RAS or mutant B-RAF, in the absence of mutant PIK3CA, correlates with sensitivity of tumor cell growth to an IGF-1R kinase inhibitor, and thus this mutant gene biomarker signature can be used as a predictor of tumor cell sensitivity to IGF-1R kinase inhibitors (e.g. OSI-906). Thus, these observations can form the basis of valuable new diagnostic methods for predicting the effects of IGF-1R kinase inhibitors on tumor growth, and give oncologists additional biomarkers to assist them in choosing the most appropriate treatment for their patients.

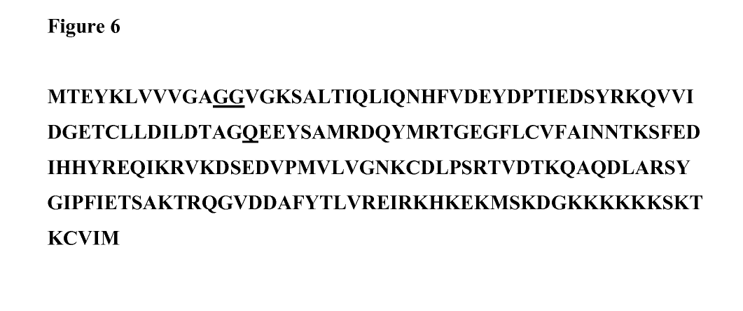

The “mutant K-RAS gene” as described herein refers to a human K-RAS gene (GeneID: 3845; encoding, for example, a protein with NCBI Accession number NP_{—}004976; see FIG. 6) with an activating mutation at any of the known KRAS activating point mutation sites. Certain exemplary activating mutations include any of those in codons 12, 13, and 61, including, but not limited to, the activating mutations: G12D (e.g. GGT>GAT), G12A (e.g. GGT>GCT), G12V (e.g. GGT>GTT), G12S (e.g. GGT>AGT), G12R (e.g. GGT>CGT), G12C (e.g. GGT>TGT), G13D (e.g. GGC>GAC), Q61H and Q61K). In one embodiment, the mutant KRAS gene has one activating mutation. In an alternative embodiment, the mutant KRAS gene has one or more activating mutations, e.g. one, two, three, or four activating mutations. Certain exemplary mutant K-RAS proteins expressed from this gene include, but are not limited to, allelic variants, splice variants, and other natural variants expressed by cells.

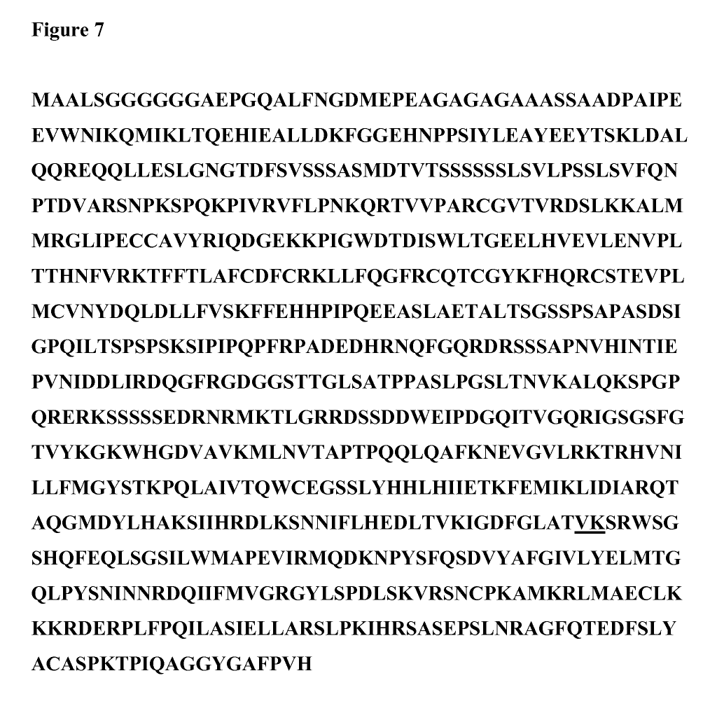

The “mutant B-RAF gene” as described herein refers to a human B-RAF gene (GeneID: 673; encoding, for example, a protein with NCBI Accession number NP_{—}004324; see FIG. 7) with an activating mutation at any of the known BRAF activating point mutation sites. Certain exemplary activating mutations include any of those in codons 600 and 601, including, but not limited to, the activating mutations V600E (e.g. T1799A), V600G (e.g. T1799G), V600A (e.g. T1799C), V600R, V600D, V600K, K601N, and K601E. In one embodiment, the mutant BRAF gene has one activating mutation. In an alternative embodiment, the mutant BRAF gene has one or more activating mutations, e.g. one, two, three, or four activating mutations. Certain exemplary mutant B-RAF proteins expressed from this gene include, but are not limited to, allelic variants, splice variants, and other natural variants expressed by cells.

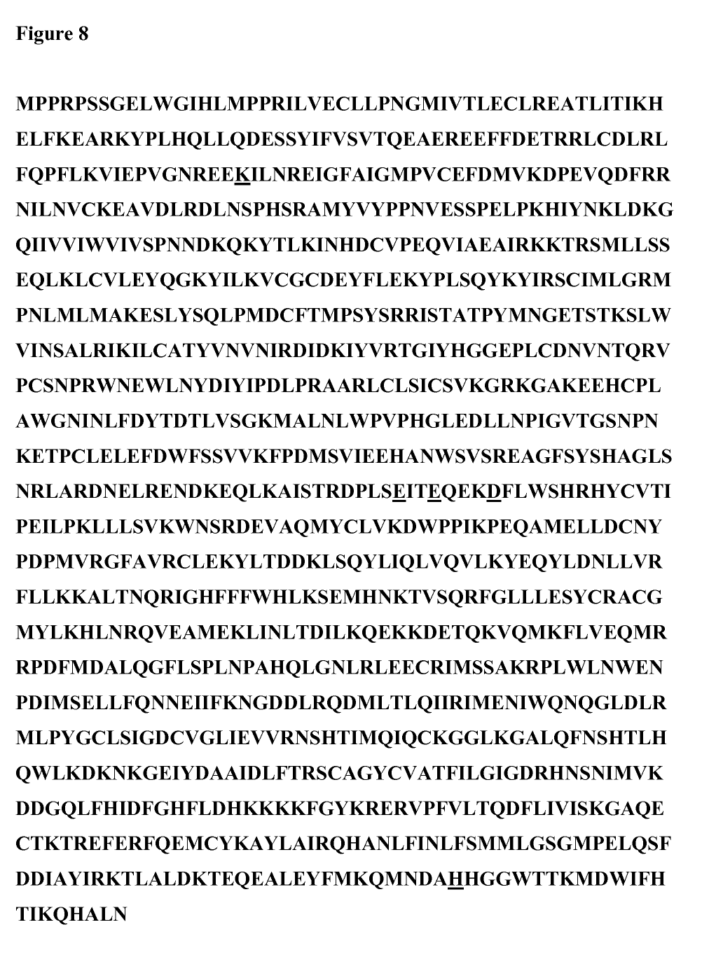

The “mutant PIK3CA gene” as described herein refers to a human PIK3CA gene (GeneID: 5290; encoding, for example, a protein with NCBI Accession number NP_{—}006209, also known as the phosphatidylinositol 3-kinase 110 kDa catalytic subunit, or p110-alpha; see FIG. 8) with an activating mutation at any of the known PIK3CA activating point mutation sites. Certain exemplary activating mutations include any of those in codons 111, 542, 545, 549, and 1047, including, but not limited to, the activating mutations E542K (e.g. G1624A), E545K (e.g. G1633A), E545G (e.g. A1634C), E545D (e.g. G1635T), H1047R (e.g. A3140G), H1047L (e.g. A3140T), K111N, K111E, and D549N. In one embodiment, the mutant PIK3CA gene has one activating mutation. In an alternative embodiment, the mutant PIK3CA gene has one or more activating mutations, e.g. one, two, three, or four activating mutations. Certain exemplary mutant PIK3CA proteins expressed from this gene include, but are not limited to, allelic variants, splice variants, and other natural variants expressed by cells.

The “mutant PTEN gene” as described herein refers to a human PTEN gene (GeneID: 5728; encoding, for example, a protein with NCBI Accession number NP_{—}000305, also known as the phosphatase and tensin homolog, MMAC1, or PTEN1) with a mutation that inactivates or reduces the activity of the enzyme in cells.

The term “activating mutation” refers to a mutation that results in a constitutively active protein. Such a mutation may cause the signal transduction pathway in which the protein is involved to be continuously active, even without extracellular stimulation by, for example, binding of an activating ligand(s) to a transmembrane receptor.

The terminology “X#Y” in the context of a mutation in a polypeptide sequence is art-recognized, where “#” indicates the location of the mutation in terms of the amino acid number of the polypeptide, “X” indicates the amino acid found at that position in the wild-type protein sequence, and “Y” indicates the amino acid at that position in the mutant protein. For example, the notation “V600E” with reference to the B-RAF polypeptide indicates that there is a valine at amino acid number 600 of the wild-type B-RAF sequence, and that valine is replaced with a glutamic acid in the mutant B-RAF sequence. One or three letter amino acid codes may be used. A similar terminology is also used to indicate the location of the mutation in the encoding nucleic acid sequence, and the change in nucleotide. The numbering of amino acids of the KRAS, BRAF and PIK3CA polypeptides is that used in NCBI databases, and amino acid residues at codons where mutations are found are indicated in FIGS. 6-8.

Thus, in any methods of the instant invention, the mutant K-RAS gene may be a human K-RAS gene with an activating mutation at any of the known KRAS activating point mutation sites. In an alternative embodiment, the mutant K-RAS gene is a human K-RAS gene with an activating mutation in codon 12, 13, or 61. In a further embodiment, the mutant K-RAS gene is a human K-RAS gene with an activating mutation in codon 12. In a further embodiment, the mutant K-RAS gene is a human K-RAS gene with an activating mutation selected from G12D, G12A, G12V, G12S, G12R, G12C, G13D, Q61H or Q61K. In another embodiment, the mutant K-RAS gene is a human K-RAS gene with an activating mutation selected from G12A, G12V, G12C, G13D, or Q61H. In another embodiment, the mutant K-RAS gene is a human K-RAS gene with the activating mutation G12V.

Thus, in any methods of the instant invention, the mutant B-RAF gene may be a human B-RAF gene with an activating mutation at any of the known B-RAF activating point mutation sites. In an alternative embodiment, the mutant B-RAF gene is a human B-RAF gene with an activating mutation in codon 600 or 601. In a further embodiment, the mutant B-RAF gene is a human B-RAF gene with an activating mutation selected from V600E, V600G, V600A, V600R, V600D, V600K, K601N, or K601E. In another embodiment, the mutant B-RAF gene is a human B-RAF gene with an activating mutation selected from V600E or K601N. In another embodiment, the mutant B-RAF gene is a human B-RAF gene with the activating mutation V600E.

Thus, in any methods of the instant invention, the mutant PIK3CA gene may be a human PIK3CA gene with an activating mutation at any of the known PIK3CA activating point mutation sites. In an alternative embodiment, the mutant PIK3CA gene is a human PIK3CA gene with an activating mutation in codon 111, 542, 545, 549, or 1047. In a further embodiment, the mutant PIK3CA gene is a human PIK3CA gene with an activating mutation in codon 111, 545, 549, or 1047. In a further embodiment, the mutant PIK3CA gene is a human PIK3CA gene with an activating mutation selected from E542K, E545K, E545G, E545D, H1047R, H1047L, K111N, K111E, or D549N. In another embodiment, the mutant PIK3CA gene is a human PIK3CA gene with an activating mutation selected from E545K, H1047R, K111N, K111E, or D549N.

In the context of this invention, the sensitivity of tumor cell growth to the IGF-1R kinase inhibitor OSI-906 is defined as high if the tumor cell is inhibited with an EC50 (half-maximal effective concentration) of less than 1 μM, and low (i.e. relatively resistant) if the tumor cell is inhibited with an EC50 of greater than 10 μM. Sensitivies between these values are considered intermediate. With other IGF-1R kinase inhibitors, particularly compounds of Formula I as described herein below, a qualitatively similar result is expected since they inhibit tumor cell growth by inhibiting the same signal transduction pathway, although quantitatively the EC50 values may differ depending on the relative cellular potency of the other inhibitor versus OSI-906. Thus, for example, the sensitivity of tumor cell growth to a more potent IGF-1R kinase inhibitor would be defined as high when the tumor cell is inhibited with an EC50 that is correspondingly lower. In tumor xenograft studies, using tumor cells of a variety of tumor cell types that all have high sensitivity to OSI-906 in culture in vitro, the tumors are consistently inhibited in vivo with a high percentage tumor growth inhibition (TGI) (see Experimental section herein). In contrast, in similar studies, using tumor cells that have low sensitivity to OSI-906 in culture in vitro, the tumors are inhibited in vivo with only a low pencentage tumor growth inhibition (TGI). These data indicate that sensitivity to IGF-1R kinase inhibitors such as OSI-906 in tumor cell culture is predictive of tumor sensitivity in vivo.

The term EC50 (half maximal effective concentration) refers to the concentration of compound which induces a response halfway between the baseline and maximum for the specified exposure time, and is used as a measure of the compound's potency.

The present invention thus provides a method of predicting the sensitivity of ovarian tumor cell growth to an IGF-1R kinase inhibitor, comprising: determining whether the tumor cells possess a mutant K-RAS gene; and predicting that tumor cell growth is likely to be sensitive to an IGF-1R kinase inhibitor if the tumor cells possess a mutant K-RAS gene. This method may be utilized to select a cancer patient who is predicted to benefit from therapeutic administration of an IGF-1R kinase inhibitor, by applying it to a sample of the cells of a tumor of the patient (e.g. a tumor biopsy, or circulating tumor cells isolated from a blood sample), either alone, or in addition to other diagnostic tests to predict response to administration of an IGF-1R kinase inhibitor. The present invention thus provides a method of identifying patients with ovarian cancer who are most likely to benefit from treatment with an IGF-1R kinase inhibitor, comprising: obtaining a sample of a patient's tumor; determining whether the tumor cells possess a mutant K-RAS gene; and identifying the patient as one most likely to benefit from treatment with an IGF-1R kinase inhibitor if the tumor cells possess a mutant K-RAS gene. Inherent in this method is the recognition that presence of a mutant KRAS gene in ovarian tumor cells correlates with higher sensitivity of the tumor cells to growth inhibition by an IGF-1R kinase inhibitor than ovarian tumor cells that have wild type KRAS.

The present invention thus provides a method of predicting the sensitivity of ovarian tumor cell growth to inhibition by an IGF-1R kinase inhibitor, comprising: determining if the ovarian tumor cells possess a mutant K-RAS gene; and concluding that if the tumor cells possess mutant K-ras, high sensitivity to growth inhibition by IGF-1R kinase inhibitors is predicted, based upon a predetermined correlation of the presence of mutant K-ras with high sensitivity.

The present invention thus provides method for treating ovarian cancer in a patient, comprising the steps of: predicting the sensitivity of ovarian tumor cell growth to inhibition by an IGF-1R kinase inhibitor, by determining if the ovarian tumor cells possess a mutant K-RAS gene; and concluding that if the tumor cells possess mutant K-ras, high sensitivity to growth inhibition by IGF-1R kinase inhibitors is predicted, based upon a predetermined correlation of the presence of mutant K-ras with high sensitivity; and administering to said patient a therapeutically effective amount of an IGF-1R kinase inhibitor if high sensitivity of the ovarian tumor cells to growth inhibition by IGF-1R kinase inhibitors is predicted.

The present invention also provides a method of identifying patients with ovarian cancer who are most likely to benefit from treatment with an IGF-1R kinase inhibitor, comprising: determining whether the ovarian tumor cells possess a mutant K-RAS gene; and identifying the patient as one most likely to benefit from treatment with an IGF-1R kinase inhibitor if the ovarian tumor cells possess a mutant K-RAS gene.

The present invention also provides a method of identifying patients with ovarian cancer who are most likely to benefit from treatment with an IGF-1R kinase inhibitor, comprising: obtaining a sample of a patient's tumor, determining if tumor cells of the sample possess a mutant K-RAS gene; and identifying the patient as likely to benefit from treatment with an IGF-1R kinase inhibitor if mutant K-ras is present in the tumor cells of the patient.

The present invention also provides a method of identifying patients with ovarian cancer who are most likely to benefit from treatment with an IGF-1R kinase inhibitor, comprising: determining whether tumor cells from a sample of a patient's tumor possess a mutant K-RAS gene; and identifying the patient as one most likely to benefit from treatment with an IGF-1R kinase inhibitor if the tumor cells possess a mutant K-RAS gene.

The present invention also provides a method for treating ovarian tumors or tumor metastases in a patient, comprising the steps of: diagnosing a patient's likely responsiveness to an IGF-1R kinase inhibitor by determining if the ovarian tumor cells of the patient possess a mutant K-RAS gene, identifying the patient as likely to benefit from treatment with an IGF-1R kinase inhibitor if mutant K-ras is present in the ovarian tumor cells of the patient, and administering to said patient a therapeutically effective amount of an IGF-1R kinase inhibitor.

The present invention also provides a method of predicting whether a patient with ovarian cancer will be responsive to treatment with an IGF-1R kinase inhibitor, comprising determining the presence or absence of a K-ras mutation in a tumor of the patient, wherein the K-ras mutation is in codon 12 or codon 13; and wherein if a K-ras mutation is present, the patient is predicted to be responsive to treatment with an IGF-1R kinase inhibitor.

The invention further provides a method for treating ovarian cancer in a patient, comprising the steps of: (A) diagnosing a patient's likely responsiveness to an IGF-1R kinase inhibitor by determining if the patient has a tumor that is likely to respond to treatment with an IGF-1R kinase inhibitor by: obtaining a sample of the patient's tumor; determining whether the tumor cells possess a mutant K-RAS gene; and identifying the patient as likely to benefit from treatment with an IGF-1R kinase inhibitor if the tumor cells possess a mutant K-RAS gene, and (B) administering to said patient a therapeutically effective amount of an IGF-1R kinase inhibitor if the patient is diagnosed to be potentially responsive to an IGF-1R kinase inhibitor.

The invention further provides a method of identifying patients with ovarian cancer who are most likely to benefit from treatment with an IGF-1R kinase inhibitor in combination with a chemotherapeutic agent, comprising: obtaining a sample of a patient's tumor, determining if tumor cells of the sample possess a mutant K-RAS gene; and identifying the patient as likely to benefit from treatment with an IGF-1R kinase inhibitor in combination with a chemotherapeutic agent if the tumor cells possess a mutant KRAS gene.

The invention further provides a method for treating ovarian cancer in a patient, comprising the steps of: (A) diagnosing a patient's likely responsiveness to an IGF-1R kinase inhibitor in combination with a chemotherapeutic agent, by determining if the patient has a tumor that is likely to respond to treatment with an IGF-1R kinase inhibitor in combination with a chemotherapeutic agent by: obtaining a sample of the patient's tumor; determining whether the tumor cells possess a mutant K-RAS gene; and identifying the patient as likely to benefit from treatment with an IGF-1R kinase inhibitor in combination with a chemotherapeutic agent if the tumor cells possess a mutant K-RAS gene, and (B) administering to said patient a therapeutically effective amount of an IGF-1R kinase inhibitor in combination with a chemotherapeutic agent if the patient is diagnosed to be potentially responsive to an IGF-1R kinase inhibitor in combination with a chemotherapeutic agent.

The chemotherapeutic agent of any of the methods of this invention which comprise a step of “identifying patients with ovarian cancer who are most likely to benefit from treatment with an IGF-1R kinase inhibitor in combination with a chemotherapeutic agent” may be selected from the following agents: pactitaxel, docetaxel, doxorubicin, or erlotinib. Thus, in one embodiment the chemotherapeutic agent is paclitaxel or docetaxel. In another embodiment the chemotherapeutic agent is doxorubicin. In another embodiment the chemotherapeutic agent is erlotinib.

The present invention further provides a method for treating ovarian tumors or tumor metastases in a patient, comprising the steps of diagnosing a patient's likely responsiveness to an IGF-1R kinase inhibitor using any of the methods described herein for determining the presence of mutant KRAS, and administering to said patient a therapeutically effective amount of an IGF-1R kinase inhibitor. For this method, an example of a preferred IGF-1R kinase inhibitor is OSI-906, or a compound with similar characteristics (e.g. selectivity, potency), including pharmacologically acceptable salts or polymorphs thereof. In this method one or more additional anti-cancer agents or treatments can be co-administered simultaneously or sequentially with the IGF-1R kinase inhibitor, as judged to be appropriate by the administering physician given the prediction of the likely responsiveness of the patient to an IGF-1R kinase inhibitor, combined with any additional circumstances pertaining to the individual patient.

It will be appreciated by one of skill in the medical arts that the exact manner of administering to a patient with ovarian cancer, a therapeutically effective amount of an IGF-1R kinase inhibitor following a diagnosis of a patient's likely responsiveness to an IGF-1R kinase inhibitor, will be at the discretion of the attending physician. The mode of administration, including dosage, combination with other anti-cancer agents, timing and frequency of administration, and the like, may be affected by the diagnosis of a patient's likely responsiveness to an IGF-1R kinase inhibitor, as well as the patient's condition and history. Thus, even patients diagnosed with ovarian tumors predicted to be relatively insensitive to IGF-1R kinase inhibitors may still benefit from treatment with such inhibitors, particularly in combination with other anti-cancer agents, or agents that may alter a tumor's sensitivity to IGF-1R kinase inhibitors.

The present invention further provides a method for treating ovarian tumors or tumor metastases in a patient, comprising the steps of diagnosing a patient's likely responsiveness to an IGF-1R kinase inhibitor by assessing whether the tumor cells are sensitive to inhibition by an IGF-1R kinase inhibitor, by for example any of the methods described herein for determining the presence of mutant KRAS in tumor cells, identifying the patient as one who is likely to demonstrate an effective response to treatment with an IGF-1R kinase inhibitor, and administering to said patient a therapeutically effective amount of an IGF-1R kinase inhibitor. In one embodiment the IGF-1R kinase inhibitor used for treatment comprises OSI-906.

The present invention also provides a method for inhibiting ovarian tumor cell growth in a patient, comprising the steps of diagnosing a patient's likely responsiveness to an IGF-1R kinase inhibitor by using any of the methods described herein to predict the sensitivity of tumor cell growth to inhibition by an IGF-1R kinase inhibitor, identifying the patient as one who is likely to demonstrate an effective response to treatment with an IGF-1R kinase inhibitor, and administering to said patient a therapeutically effective amount of an IGF-1R kinase inhibitor. In one embodiment the IGF-1R kinase inhibitor used for treatment comprises OSI-906.

The present invention further provides a method for treating ovarian tumors or tumor metastases in a patient, comprising the steps of diagnosing a patient's likely responsiveness to an IGF-1R kinase inhibitor by any of the methods described herein for determining mutant KRAS biomarkers, identifying the patient as one who is less likely or not likely to demonstrate an effective response to treatment with an IGF-1R kinase inhibitor, and treating said patient with an anti-cancer therapy other than an IGF-1R kinase inhibitor. In one embodiment of this method, the anti-cancer therapy other than an IGF-1R kinase inhibitor is a standard treatment for ovarian cancer, e.g. paclitaxel in combination with either cisplatin or carboplatin.

The present invention provides for any of the methods of identifying patients with cancer who are most likely to benefit from treatment with an IGF-1R kinase inhibitor described herein, the method as described but including an additional step of assessment of the level of IGF-1 and/or IGF-2 (i.e. insulin-like growth factors 1 and/or 2) in the tumor of the patient. The present invention also provides for any of the methods of treatment with an IGF-1R kinase inhibitor described herein, the method as described but including prior to the step of administering to the patient an IGF-1R kinase inhibitor, an additional step of assessment of the level of IGF-1 and/or IGF-2 (i.e. insulin-like growth factors 1 and/or 2) in the tumor of the patient. Since IGF-1R has been reported to be activated only upon ligand (i.e. IGF-1 and/or IGF-2) binding, if there is no IGF-1R ligand present in a tumor, then even if one or more of the methods of the instant invention predict that it should be sensitive to inhibition by IGF-1R kinase inhibitors, the tumor cells cannot under such circumstances be relying on the IGF-1R signaling pathway for growth and survival, and thus an IGF-1R kinase inhibitor would probably not be an effective treatment. Many tumors have been found to express elevated levels of IGF-1 and/or IGF-2 (Pollack, M. N. et al. (2004) Nature Reviews Cancer 4:505-518), which could originate from the tumor cells themselves, from stromal cells present in the tumor, or via the vascular system from non-tumor cells (e.g. liver cells). Assessment of the level of IGF-1 and/or IGF-2 can be performed by any method known in the art, such as for example any of the methods described herein for assessment of biomarkers levels, e.g. immunoassay determination of IGF-1 and/or IGF-2 protein levels; determination of IGF-1 and/or IGF-2 mRNA transcript levels. In an alternative embodiment, the of step of assessment of the level of IGF-1 and/or IGF-2 (i.e. insulin-like growth factors 1 and/or 2) in the tumor of the patient can be replaced with a step of assessment of the level of IGF-1 and/or IGF-2 (i.e. insulin-like growth factors 1 and/or 2) in the blood or serum of the patient. This alternative, though not a direct measure of the level of IGF-1 and/or IGF-2 in the tumor, can give an indication of the potential availability of ligand to the IGF-1R in the tumor, and is a simpler and less expensive test. The potential disadvantage of this indirect assessment of IGF-1 and/or IGF-2 is that it may not give a true indication of the levels of ligand in the tumor if IGF-1 and/or IGF-2 is produced locally in the tumor, either by the tumor cells themselves, or by stromal cells within the tumor. In these methods with the additional step of assessment of the level of IGF-1 and/or IGF-2, the presence of IGF-1 and/or IGF-2 is an additional condition required for identifying the patient as likely to benefit from treatment with an IGF-1R kinase inhibitor, or to be diagnosed to be potentially responsive to an IGF-1R kinase inhibitor, and thus required prior to administering to said patient a therapeutically effective amount of an IGF-1R kinase inhibitor.

Accordingly, the invention provides a method of identifying patients with ovarian cancer who are most likely to benefit from treatment with an IGF-1R kinase inhibitor, comprising: obtaining a sample of a patient's tumor; determining whether the tumor cells possess a mutant K-RAS gene; assessing whether IGF-1 and/or IGF-2 is present in the tumor; and identifying the patient as one most likely to benefit from treatment with an IGF-1R kinase inhibitor if the tumor cells possess a mutant K-RAS gene and IGF-1 and/or IGF-2 is present.

The invention also provides a method for treating ovarian tumors or tumor metastases in a patient, comprising the steps of: diagnosing a patient's likely responsiveness to an IGF-1R kinase inhibitor, by determining the presence or absence of mutant KRAS in the tumor cells, wherein the presence of mutant KRAS correlates with high sensitivity to inhibition by IGF-1R kinase inhibitors; assessing the level of IGF-1 and/or IGF-2 in the tumor (or blood or serum) of the patient; and administering to said patient a therapeutically effective amount of an IGF-1R kinase inhibitor if the patient is diagnosed to be potentially responsive to an IGF-1R kinase inhibitor, and IGF-1 and/or IGF-2 is determined to be present in the tumor (or blood or serum levels indicate the potential availability of IGF-1 and/or IGF-2 to the tumor cells). In one embodiment the presence of IGF-1 and/or IGF-2 in the tumor is determined by assessing the level of IGF-1 and/or IGF-2 protein in the tumor cells (e.g. by immunohistochemistry). In another embodiment the presence of IGF-1 and/or IGF-2 in the tumor is determined by assessing the level of IGF-1 and/or IGF-2 RNA transcripts in the tumor cells (e.g. by quantitative RT-PCR).

The invention also provides a method for treating ovarian tumors or tumor metastases in a patient, comprising the steps of: diagnosing a patient's likely responsiveness to an IGF-1R kinase inhibitor, by determining whether the tumor cells possess a mutant K-RAS gene and assessing whether IGF-1 and/or IGF-2 is present in the tumor; and administering to said patient a therapeutically effective amount of an IGF-1R kinase inhibitor if the patient is diagnosed to be potentially responsive to an IGF-1R kinase inhibitor by having tumor cells that possess a mutant KRAS gene and the presence of IGF-1 and/or IGF-2 in the tumor.

The invention also provides a method of identifying patients with ovarian cancer who are most likely to benefit from treatment with an IGF-1R kinase inhibitor, comprising: obtaining a sample of a patient's tumor; determining whether the tumor cells possess a mutant K-RAS gene; assessing whether IGF-1 and/or IGF-2 is present in the tumor; and identifying the patient as one most likely to benefit from treatment with an IGF-1R kinase inhibitor if the tumor cells possess a mutant K-RAS gene and IGF-1 and/or IGF-2 is present in the tumor.

The invention also provides a method for treating ovarian cancer in a patient, comprising the steps of: (A) diagnosing a patient's likely responsiveness to an IGF-1R kinase inhibitor by determining if the patient has an ovarian tumor that is likely to respond to treatment with an IGF-1R kinase inhibitor by: obtaining a sample of the patient's tumor; determining whether the tumor cells possess a mutant K-RAS gene and assessing whether IGF-1 and/or IGF-2 is present in the tumor; and identifying the patient as likely to benefit from treatment with an IGF-1R kinase inhibitor if the tumor cells possess a mutant K-RAS gene and IGF-1 and/or IGF-2 is present in the tumor, and (B) administering to said patient a therapeutically effective amount of an IGF-1R kinase inhibitor if the patient is diagnosed to be potentially responsive to an IGF-1R kinase inhibitor by having tumor cells that possess a mutant KRAS gene and the presence of IGF-1 and/or IGF-2 in the tumor.

The effectiveness of treatment in the preceding methods can be determined for example by measuring the decrease in size of the ovarian tumors present in the patients, or a biomarker that correlates with the presence of ovarian tumor cells, or by assaying a molecular determinant of the degree of proliferation of the ovarian tumor cells.

The invention provides a method of identifying patients with cancer who are most likely to benefit from treatment with an IGF-1R kinase inhibitor, comprising: obtaining a sample of a patient's tumor; determining if tumor cells of the sample possess a mutant K-RAS gene; determining if tumor cells of the sample possess a mutant PIK3CA gene; and identifying the patient as likely to benefit from treatment with an IGF-1R kinase inhibitor if mutant K-ras is present in the tumor cells of the patient in the absence of mutant PIK3CA.

The invention also provides a method for treating cancer in a patient, comprising the steps of: (A) diagnosing a patient's likely responsiveness to an IGF-1R kinase inhibitor by determining if the patient has a tumor that is likely to respond to treatment with an IGF-1R kinase inhibitor by: obtaining a sample of the patient's tumor; determining if tumor cells of the sample possess a mutant K-RAS gene; determining if tumor cells of the sample possess a mutant PIK3CA gene; and identifying the patient as likely to benefit from treatment with an IGF-1R kinase inhibitor if mutant K-ras is present in the tumor cells of the patient in the absence of mutant PIK3CA; and (B) administering to said patient a therapeutically effective amount of an IGF-1R kinase inhibitor if the patient is diagnosed to be potentially responsive to an IGF-1R kinase inhibitor.

The invention also provides a method of identifying patients with cancer who are most likely to benefit from treatment with an IGF-1R kinase inhibitor, comprising: obtaining a sample of a patient's tumor, determining if tumor cells of the sample possess a mutant B-RAF gene; determining if tumor cells of the sample possess a mutant PIK3CA gene; and identifying the patient as likely to benefit from treatment with an IGF-1R kinase inhibitor if mutant B-RAF is present in the tumor cells of the patient in the absence of mutant PIK3CA.

The present invention also provides a method of identifying patients with cancer who are most likely to benefit from treatment with an IGF-1R kinase inhibitor, comprising: determining if tumor cells from a sample of a patient's tumor possess a mutant K-RAS gene or a mutant B-RAF gene; determining if tumor cells of the sample possess a mutant PIK3CA gene; and identifying the patient as likely to benefit from treatment with an IGF-1R kinase inhibitor if mutant K-ras or mutant B-RAF is present in the tumor cells of the patient in the absence of mutant PIK3CA.

The invention provides a method of identifying patients with cancer who are most likely to benefit from treatment with an IGF-1R kinase inhibitor, comprising: obtaining a sample of a patient's tumor; determining if tumor cells of the sample possess a mutant PIK3CA gene; and identifying the patient as likely to benefit from treatment with an IGF-1R kinase inhibitor if mutant PIK3CA is not present in the tumor cells of the patient.

The invention provides a method for treating cancer in a patient, comprising the steps of: (A) diagnosing a patient's likely responsiveness to an IGF-1R kinase inhibitor by determining if the patient has a tumor that is likely to respond to treatment with an IGF-1R kinase inhibitor by: obtaining a sample of a patient's tumor; determining if tumor cells of the sample possess a mutant PIK3CA gene; and identifying the patient as likely to benefit from treatment with an IGF-1R kinase inhibitor if mutant PIK3CA is not present in the tumor cells of the patient; and (B) administering to said patient a therapeutically effective amount of an IGF-1R kinase inhibitor if the patient is diagnosed to be potentially responsive to an IGF-1R kinase inhibitor.

The invention also provides a method for treating cancer in a patient, comprising the steps of: (A) diagnosing a patient's likely responsiveness to an IGF-1R kinase inhibitor by determining if the patient has a tumor that is likely to respond to treatment with an IGF-1R kinase inhibitor by: obtaining a sample of the patient's tumor; determining if tumor cells of the sample possess a mutant B-RAF gene; determining if tumor cells of the sample possess a mutant PIK3CA gene; and identifying the patient as likely to benefit from treatment with an IGF-1R kinase inhibitor if mutant B-RAF is present in the tumor cells of the patient in the absence of mutant PIK3CA; and (B) administering to said patient a therapeutically effective amount of an IGF-1R kinase inhibitor if the patient is diagnosed to be potentially responsive to an IGF-1R kinase inhibitor.

The invention also provides a method of identifying patients with cancer who are most likely to benefit from treatment with an IGF-1R kinase inhibitor, comprising: obtaining a sample of a patient's tumor, determining if tumor cells of the sample possess a mutant K-RAS gene; determining if tumor cells of the sample possess a mutant B-RAF gene; determining if tumor cells of the sample possess a mutant PIK3CA gene; and identifying the patient as likely to benefit from treatment with an IGF-1R kinase inhibitor if mutant K-ras or mutant B-RAF is present in the tumor cells of the patient in the absence of mutant PIK3CA.

The invention also provides a method for treating cancer in a patient, comprising the steps of: (A) diagnosing a patient's likely responsiveness to an IGF-1R kinase inhibitor by determining if the patient has a tumor that is likely to respond to treatment with an IGF-1R kinase inhibitor by: obtaining a sample of the patient's tumor, determining if tumor cells of the sample possess a mutant K-RAS gene; determining if tumor cells of the sample possess a mutant B-RAF gene; determining if tumor cells of the sample possess a mutant PIK3CA gene; and identifying the patient as likely to benefit from treatment with an IGF-1R kinase inhibitor if mutant K-ras or mutant B-RAF is present in the tumor cells of the patient in the absence of mutant PIK3CA; and (B) administering to said patient a therapeutically effective amount of an IGF-1R kinase inhibitor if the patient is diagnosed to be potentially responsive to an IGF-1R kinase inhibitor.

The invention also provides a method of identifying patients with cancer who are most likely to benefit or not benefit from treatment with an IGF-1R kinase inhibitor, comprising: obtaining a sample of a patient's tumor, determining if tumor cells of the sample possess a mutant K-RAS gene; determining if tumor cells of the sample possess a mutant B-RAF gene; determining if tumor cells of the sample possess a mutant PIK3CA gene; and identifying the patient as likely to benefit from treatment with an IGF-1R kinase inhibitor if mutant K-ras or mutant B-RAF is present in the tumor cells of the patient in the absence of mutant PIK3CA; and identifying the patient as likely to not benefit from treatment with an IGF-1R kinase inhibitor if mutant PIK3CA is present in the tumor cells of the patient.

The invention also provides a method for treating cancer in a patient, comprising the steps of: (A) diagnosing a patient's likely responsiveness to an IGF-1R kinase inhibitor by determining if the patient has a tumor that is likely to respond to treatment with an IGF-1R kinase inhibitor by: obtaining a sample of the patient's tumor, determining if tumor cells of the sample possess a mutant K-RAS gene; determining if tumor cells of the sample possess a mutant B-RAF gene; determining if tumor cells of the sample possess a mutant PIK3CA gene; and identifying the patient as likely to benefit from treatment with an IGF-1R kinase inhibitor if mutant K-ras or mutant B-RAF is present in the tumor cells of the patient in the absence of mutant PIK3CA; and identifying the patient as likely to not benefit from treatment with an IGF-1R kinase inhibitor if mutant PIK3CA is present in the tumor cells of the patient; and (B) administering to said patient a therapeutically effective amount of an IGF-1R kinase inhibitor if the patient is diagnosed to be potentially responsive to an IGF-1R kinase inhibitor.

For any of the methods described herein involving determining if tumor cells of the sample possess a mutant K-RAS or B-RAF gene, and a mutant PIK3CA gene, to assess a patient's likely responsiveness to an IGF-1R kinase inhibitor, this invention also provides a corresponding method to assess a patient's likely responsiveness to a combination of an IGF-1R kinase inhibitor and a chemotherapeutic agent, and method of treatment with a combination of an IGF-1R kinase inhibitor and a chemotherapeutic agent. For example, the invention provides a method of identifying patients with cancer who are most likely to benefit from treatment with a combination of an IGF-1R kinase inhibitor and a chemotherapeutic agent, comprising: obtaining a sample of a patient's tumor, determining if tumor cells of the sample possess a mutant K-RAS gene; determining if tumor cells of the sample possess a mutant B-RAF gene; determining if tumor cells of the sample possess a mutant PIK3CA gene; and identifying the patient as likely to benefit from treatment with a combination of an IGF-1R kinase inhibitor and a chemotherapeutic agent if mutant K-ras or mutant B-RAF is present in the tumor cells of the patient in the absence of mutant PIK3CA. The invention also provides a method for treating cancer in a patient, comprising the steps of: (A) diagnosing a patient's likely responsiveness to a combination of an IGF-1R kinase inhibitor and a chemotherapeutic agent by determining if the patient has a tumor that is likely to respond to treatment with an a combination of an IGF-1R kinase inhibitor and a chemotherapeutic agent by: obtaining a sample of the patient's tumor, determining if tumor cells of the sample possess a mutant K-RAS gene; determining if tumor cells of the sample possess a mutant B-RAF gene; determining if tumor cells of the sample possess a mutant PIK3CA gene; and identifying the patient as likely to benefit from treatment with a combination of an IGF-1R kinase inhibitor and a chemotherapeutic agent if mutant K-ras or mutant B-RAF is present in the tumor cells of the patient in the absence of mutant PIK3CA; and (B) administering to said patient a therapeutically effective amount of a combination of an IGF-1R kinase inhibitor and a chemotherapeutic agent if the patient is diagnosed to be potentially responsive to a combination of an IGF-1R kinase inhibitor and a chemotherapeutic agent.

The invention also provides a method of identifying patients with cancer who are most likely to benefit from treatment with an IGF-1R kinase inhibitor, comprising: obtaining a sample of a patient's tumor, determining if tumor cells of the sample possess a mutant K-RAS gene; determining if tumor cells of the sample possess a mutant B-RAF gene; determining if tumor cells of the sample possess a mutant PIK3CA gene; assessing whether IGF-1 and/or IGF-2 is present in the tumor; and identifying the patient as likely to benefit from treatment with an IGF-1R kinase inhibitor if mutant K-ras or mutant B-RAF is present in the tumor cells of the patient in the absence of mutant PIK3CA, and IGF-1 and/or IGF-2 is present in the tumor.

The invention also provides a method for treating cancer in a patient, comprising the steps of: (A) diagnosing a patient's likely responsiveness to an IGF-1R kinase inhibitor by determining if the patient has a tumor that is likely to respond to treatment with an IGF-1R kinase inhibitor by: obtaining a sample of the patient's tumor, determining if tumor cells of the sample possess a mutant K-RAS gene; determining if tumor cells of the sample possess a mutant B-RAF gene; determining if tumor cells of the sample possess a mutant PIK3CA gene; assessing whether IGF-1 and/or IGF-2 is present in the tumor; and identifying the patient as likely to benefit from treatment with an IGF-1R kinase inhibitor if mutant K-ras or mutant B-RAF is present in the tumor cells of the patient in the absence of mutant PIK3CA, and IGF-1 and/or IGF-2 is present in the tumor; and (B) administering to said patient a therapeutically effective amount of an IGF-1R kinase inhibitor if the patient is diagnosed to be potentially responsive to an IGF-1R kinase inhibitor.

The invention also provides a method of identifying patients with cancer who are most likely to benefit or not benefit from treatment with an IGF-1R kinase inhibitor, comprising: obtaining a sample of a patient's tumor, determining if tumor cells of the sample possess a mutant K-RAS gene; determining if tumor cells of the sample possess a mutant B-RAF gene; determining if tumor cells of the sample possess a mutant PIK3CA gene; assessing whether IGF-1 and/or IGF-2 is present in the tumor; and identifying the patient as likely to benefit from treatment with an IGF-1R kinase inhibitor if mutant K-ras or mutant B-RAF is present in the tumor cells of the patient in the absence of mutant PIK3CA, and IGF-1 and/or IGF-2 is present in the tumor; and identifying the patient as likely to not benefit from treatment with an IGF-1R kinase inhibitor if mutant PIK3CA is present in the tumor cells of the patient.

The invention also provides a method for treating cancer in a patient, comprising the steps of: (A) diagnosing a patient's likely responsiveness to an IGF-1R kinase inhibitor by determining if the patient has a tumor that is likely to respond to treatment with an IGF-1R kinase inhibitor by: obtaining a sample of the patient's tumor, determining if tumor cells of the sample possess a mutant K-RAS gene; determining if tumor cells of the sample possess a mutant B-RAF gene; determining if tumor cells of the sample possess a mutant PIK3CA gene; assessing whether IGF-1 and/or IGF-2 is present in the tumor; and identifying the patient as likely to benefit from treatment with an IGF-1R kinase inhibitor if mutant K-ras or mutant B-RAF is present in the tumor cells of the patient in the absence of mutant PIK3CA, and IGF-1 and/or IGF-2 is present in the tumor; and identifying the patient as likely to not benefit from treatment with an IGF-1R kinase inhibitor if mutant PIK3CA is present in the tumor cells of the patient; and (B) administering to said patient a therapeutically effective amount of an IGF-1R kinase inhibitor if the patient is diagnosed to be potentially responsive to an IGF-1R kinase inhibitor.

The invention provides a method of predicting the sensitivity of tumor cell growth to inhibition by an IGF-1R kinase inhibitor, comprising: determining if the tumor cells possess a mutant K-RAS gene; determining if the tumor cells possess a mutant PIK3CA gene; and concluding that if the tumor cells possess mutant K-RAS, in the absence of mutant PIK3CA, high sensitivity to growth inhibition by an IGF-1R kinase inhibitor is predicted, based upon a predetermined correlation of the presence of mutant K-RAS in the absence of mutant PIK3CA with high sensitivity.

The invention provides a method for treating a patient with a tumor, comprising the steps of: predicting the sensitivity of tumor cell growth to inhibition by an IGF-1R kinase inhibitor, by determining if the tumor cells possess a mutant K-RAS gene; determining if the tumor cells possess a mutant PIK3CA gene; and concluding that if the tumor cells possess mutant K-RAS, in the absence of mutant PIK3CA, high sensitivity to growth inhibition by an IGF-1R kinase inhibitor is predicted, based upon a predetermined correlation of the presence of mutant K-RAS in the absence of mutant PIK3CA with high sensitivity; and administering to said patient a therapeutically effective amount of an IGF-1R kinase inhibitor if high sensitivity of the tumor cells to growth inhibition by IGF-1R kinase inhibitor is predicted.

The invention provides a method of predicting the sensitivity of tumor cell growth to inhibition by an IGF-1R kinase inhibitor, comprising: determining if the tumor cells possess a mutant B-RAF gene; determining if the tumor cells possess a mutant PIK3CA gene; and concluding that if the tumor cells possess mutant B-RAF, in the absence of mutant PIK3CA, high sensitivity to growth inhibition by an IGF-1R kinase inhibitor is predicted, based upon a predetermined correlation of the presence of mutant B-RAF in the absence of mutant PIK3CA with high sensitivity.

The invention provides a method for treating a patient with a tumor, comprising the steps of: predicting the sensitivity of tumor cell growth to inhibition by an IGF-1R kinase inhibitor, by determining if the tumor cells possess a mutant B-RAF gene; determining if the tumor cells possess a mutant PIK3CA gene; and concluding that if the tumor cells possess mutant B-RAF, in the absence of mutant PIK3CA, high sensitivity to growth inhibition by an IGF-1R kinase inhibitor is predicted, based upon a predetermined correlation of the presence of mutant B-RAF in the absence of mutant PIK3CA with high sensitivity; and administering to said patient a therapeutically effective amount of an IGF-1R kinase inhibitor if high sensitivity of the tumor cells to growth inhibition by IGF-1R kinase inhibitor is predicted.

The invention provides a method of predicting the sensitivity of tumor cell growth to inhibition by an IGF-1R kinase inhibitor, comprising: determining if the tumor cells possess a mutant PIK3CA gene; and concluding that if the tumor cells possess mutant PIK3CA, low sensitivity to growth inhibition by an IGF-1R kinase inhibitor is predicted, based upon a predetermined correlation of the presence of mutant PIK3CA with low sensitivity.

The invention provides a method for treating a patient with a tumor, comprising the steps of: predicting the sensitivity of tumor cell growth to inhibition by an IGF-1R kinase inhibitor, by determining if the tumor cells possess a mutant PIK3CA gene; and concluding that if the tumor cells possess mutant PIK3CA, low sensitivity to growth inhibition by an IGF-1R kinase inhibitor is predicted, based upon a predetermined correlation of the presence of mutant PIK3CA with low sensitivity; and administering to said patient a therapeutically effective amount of an IGF-1R kinase inhibitor if low sensitivity of the tumor cells to growth inhibition by IGF-1R kinase inhibitor is not predicted (i.e. a mutant PIK3CA gene is not found).

The invention provides a method of predicting the sensitivity of tumor cell growth to inhibition by an IGF-1R kinase inhibitor, comprising: determining if the tumor cells possess a mutant PTEN gene; and concluding that if the tumor cells possess mutant PTEN, low sensitivity to growth inhibition by an IGF-1R kinase inhibitor is predicted, based upon a predetermined correlation of the presence of mutant PTEN with low sensitivity, as described herein.

The invention provides a method of predicting the sensitivity of tumor cell growth to inhibition by an IGF-1R kinase inhibitor in a patient, comprising: determining if tumor cells from a sample of a patient's tumor possess a mutant PTEN gene or a mutant PIK3CA gene; and concluding that if the tumor cells possess mutant PTEN or mutant PIK3CA, low sensitivity to growth inhibition by an IGF-1R kinase inhibitor is predicted in the patient, based upon a predetermined correlation of the presence of mutant PTEN or mutant PIK3CA with low sensitivity, as described herein.

The invention provides a method for treating a patient with a tumor, comprising the steps of: predicting the sensitivity of tumor cell growth to inhibition by an IGF-1R kinase inhibitor, by determining if the tumor cells possess a mutant PTEN gene; and concluding that if the tumor cells possess mutant PTEN, low sensitivity to growth inhibition by an IGF-1R kinase inhibitor is predicted, based upon a predetermined correlation of the presence of mutant PTEN with low sensitivity; and administering to said patient a therapeutically effective amount of an IGF-1R kinase inhibitor if low sensitivity of the tumor cells to growth inhibition by IGF-1R kinase inhibitor is not predicted. Determining if the tumor cells possess a mutant PTEN gene may be performed on a sample of tumor cells from the patient, using for example any of the methods described herein.

The invention also provides a method for treating cancer in a patient, comprising administering to said patient a therapeutically effective amount of an IGF-1R kinase inhibitor (e.g. OSI-906) if the patient has been diagnosed to be potentially responsive to an IGF-1R kinase inhibitor by a determination that the tumor cells of the patient do not possess a mutant PTEN gene.

The invention also provides a method for treating cancer in a patient, comprising administering to said patient a therapeutically effective amount of an IGF-1R kinase inhibitor (e.g. OSI-906) if the patient has been diagnosed to be potentially responsive to an IGF-1R kinase inhibitor by a determination that the tumor cells of the patient do not possess a mutant PTEN gene or a mutant PIK3CA gene.

The invention provides a method of predicting the sensitivity of tumor cell growth to inhibition by an IGF-1R kinase inhibitor, comprising: determining if the tumor cells possess a mutant K-RAS gene; determining if the tumor cells possess a mutant B-RAF gene; determining if the tumor cells possess a mutant PIK3CA gene; and concluding that if the tumor cells possess mutant K-RAS or mutant B-RAF, in the absence of mutant PIK3CA, high sensitivity to growth inhibition by an IGF-1R kinase inhibitor is predicted, based upon a predetermined correlation of the presence of mutant K-RAS or mutant B-RAF, in the absence of mutant PIK3CA, with high sensitivity.

The invention provides a method for treating a patient with a tumor, comprising the steps of: predicting the sensitivity of tumor cell growth to inhibition by an IGF-1R kinase inhibitor, by determining if the tumor cells possess a mutant K-RAS gene; determining if the tumor cells possess a mutant B-RAF gene; determining if the tumor cells possess a mutant PIK3CA gene; and concluding that if the tumor cells possess mutant K-RAS or mutant B-RAF, in the absence of mutant PIK3CA, high sensitivity to growth inhibition by an IGF-1R kinase inhibitor is predicted, based upon a predetermined correlation of the presence of mutant K-RAS or mutant B-RAF, in the absence of mutant PIK3CA, with high sensitivity; and administering to said patient a therapeutically effective amount of an IGF-1R kinase inhibitor if high sensitivity of the tumor cells to growth inhibition by IGF-1R kinase inhibitor is predicted.

The invention also provides a method for treating cancer in a patient, comprising administering to said patient a therapeutically effective amount of an IGF-1R kinase inhibitor if the patient is diagnosed to be potentially responsive to an IGF-1R kinase inhibitor by determining that the tumor cells of the patient possess a mutant K-ras or mutant B-RAF gene in the absence of a mutant PIK3CA gene.

The invention also provides a method for treating cancer in a patient, comprising administering to said patient a therapeutically effective amount of an IGF-1R kinase inhibitor if the tumor cells of the patient possess a mutant K-ras or mutant B-RAF gene in the absence of a mutant PIK3CA gene.

The invention further provides a method for treating ovarian cancer in a patient, comprising administering to said patient a therapeutically effective amount of an IGF-1R kinase inhibitor if the patient is diagnosed to be potentially responsive to an IGF-1R kinase inhibitor by determining that the tumor cells of the patient possess a mutant K-ras gene.

The invention further provides a method for treating ovarian cancer in a patient, comprising administering to said patient a therapeutically effective amount of an IGF-1R kinase inhibitor if the tumor cells of the patient possess a mutant K-ras gene.

This invention also encompasses any of the methods of the invention described herein, wherein the step of “obtaining a sample of a patient's tumor” is omitted. In such cases, the step of determining tumor biomarker expression (e.g. mutant KRAS, BRAF, PTEN or PIK3CA) may for example be performed on a previously processed or prepared tumor sample, e.g. a frozen tumor sample, a fixed tumor preparation, a cell extract, an RNA preparation, a protein preparation, or the like, from which biomarker expression can be assessed, or a biological fluid where the tumor biomarker can be found, as an alternative to the tumor sample itself (e.g. a biopsy).

Although the experimental examples provided herein involve the IGF-1R kinase inhibitor, OSI-906, the methods of the present invention are not limited to the prediction of patients or tumors that will respond or not respond to any particular IGF-1R kinase inhibitor, but rather, can be used to predict patient's outcome to any IGF-1R kinase inhibitor, including IGF-1R kinase inhibitors that inhibit both IGF-1R and IR kinases (e.g. OSI-906 (OSI Pharmaceuticals, Inc.), BMS-554417 (Haluska P, et al. Cancer Res 2006; 66(1):362-71); BMS 536924 (Huang, F. et al. (2009) Cancer Res. 69(1):161-170), BMS-754807 (Bristol-Myers Squibb)), inhibitors that are small molecules (e.g. AXL-1717 (Axelar AB), XL-228 (Exelixus), INSM-18 (Insmed Inc.)), peptides, antibodies (e.g. IMCL-A12 (a.k.a. cixutumumab; Imclone), MK-0646 (Merck), CP-751871 (a.k.a. figitumumab; Pfizer), AMG-479 (Amgen), SCH-717454 (a.k.a. robatumumab; Schering-Plough/Merck), antibody fragments, nucleic acids, or other types of IGF-1R kinase inhibitor inhibitors. Similarly, the methods of treatment with an IGF-1R kinase inhibitor described herein may use any of these types of IGF-1R kinase inhibitor. Furthermore, in another embodiment of any of the methods described herein the IGF-1R kinase inhibitor may be an IGF-1R kinase inhibitor approved by a government regulatory authority (e.g. US Food and Drug Administration (FDA); European Medicines Agency; Japanese Ministry of Health, Labour & Welfare; UK Medicines and Healthcare Products Regulatory Agency (MHRA)) (e.g. any of the IGF-1R kinase inhibitors disclosed herein that have been so approved).

In any of the methods, compositions or kits of the invention described herein, the term “small molecule IGF-1R kinase inhibitor” refers to a low molecular weight (i.e. less than 5000 Daltons; preferably less than 1000, and more preferably between 300 and 700 Daltons) organic compound that inhibits IGF-1R kinase by binding to the kinase domain of the enzyme. Examples of such compounds include IGF-1R kinase inhibitors of Formula (I) as described herein. The IGF-1R kinase inhibitor of Formula (I) can be any IGF-1R kinase inhibitor compound encompassed by Formula (I) that inhibits IGF-1R kinase upon administration to a patient. Examples of such inhibitors have been published in US Published Patent Application US 2006/0235031, which is incorporated herein in its entirety, and include OSI-906 (cis-3-[8-amino-1-(2-phenyl-quinolin-7-yl)-imidazo[1,5-a]pyrazin-3-yl]-1-methyl-cyclobutanol), as used in the experiments described herein.

One of skill in the medical arts, particularly pertaining to the application of diagnostic tests and treatment with therapeutics, will recognize that biological systems are somewhat variable and not always entirely predictable, and thus many good diagnostic tests or therapeutics are occasionally ineffective. Thus, it is ultimately up to the judgment of the attending physician to determine the most appropriate course of treatment for an individual patient, based upon test results, patient condition and history, and his own experience. There may even be occasions, for example, when a physician will choose to treat a patient with an IGF-1R kinase inhibitor even when a tumor is not predicted to be particularly sensitive to IGF-1R kinase inhibitors, based on data from diagnostic tests or from other criteria, particularly if all or most of the other obvious treatment options have failed, or if some synergy is anticipated when given with another treatment. The fact that the IGF-1R kinase inhibitors as a class of compounds are relatively well tolerated compared to many other anti-cancer compounds, such as more traditional chemotherapy or cytotoxic agents used in the treatment of cancer, makes this a more viable option. Also, it should be noted that while the mutant biomarkers disclosed herein predict which patients with tumors are likely to receive the most benefit from IGF-1R kinase inhibitors, it does not necessarily mean that patients with tumors which do not possess a mutant biomarker signature predicting sensitivity will receive no benefit, just that a more modest effect is to be anticipated.

As described herein, this invention provides methods using mutant biomarker gene status to predict tumor sensitivity to inhibition by IGF-1R kinase inhibitors. All diagnostic methods have potential advantages and disadvantages, and while the preferred method will ultimately depend on individual patient circumstances, the use of multiple diagnostic methods will likely improve one's ability to accurately predict the likely outcome of a therapeutic regimen comprising use of an IGF-1R kinase inhibitor. Therefore, this invention also provides methods for diagnosing or for treating a patient with cancer, comprising the use of two or more diagnostic methods for predicting sensitivity to inhibition by IGF-1R kinase inhibitors, followed in the case of a treatment method by administering to said patient of a therapeutically effective amount of an IGF-1R kinase inhibitor if two or more of the diagnostic methods indicate that the patient is potentially responsive to an IGF-1R kinase inhibitor. One of the diagnostic methods for predicting sensitivity to inhibition by IGF-1R kinase inhibitors may be a method as described herein using mutant KRAS, BRAF, PTEN and/or PIK3CA gene status to predict tumor sensitivity to inhibition by IGF-1R kinase inhibitors. The other diagnostic method(s) may be any method already known in the art for using biomarkers to predict sensitivity to inhibition by IGF-1R kinase inhibitors, e.g. determination of epithelial or mesenchymal biomarker expression level to assess tumor cell EMT status (e.g. E-cadherin; US 2007/0065858; US 20090092596); biomarkers predicting sensitivity or resistance to IGF-1R kinase inhibitors as described in T. Pitts et al. (2009) EORTC Conference, Boston, Mass., abstract #2141; pERK, pHER3 or HER3 (US 2009/0093488); IGF-1, IGF-2, or other biomarkers reported to predict sensitivity to IGF-1R kinase inhibitors (e.g. see Huang F. H. W., et al. Identification of sensitivity markers for BMS-536924, an inhibitor for insulin-like growth factor-1 receptor. J Clin Oncol ASCO Ann Meet Proc Part I 2007; 25:3506).

Determination of mutant KRAS biomarker status can be assessed by a number of different approaches known in the art, including direct analysis of KRAS proteins by, for example, immunoassay using mutant KRAS specific antibodies (e.g. U.S. Pat. Nos. 5,262,523; 5,081,230; 4,898,932). An advantage of this approach is that expressed biomarkers are read directly. However, this approach also requires sufficient quantities of tissue in order to perform an analysis (e.g. immunohistochemistry). Sufficient quantities of tissue may be difficult to obtain from certain procedures such as FNA (fine needle aspiration). Core biopsies provide larger amounts of tissue, but are sometimes not routinely performed during diagnoses. Alternatively, mutant KRAS biomarker can be evaluated from DNA, or protein-encoding RNA transcripts, using a quantitative PCR based approach. An advantage of this approach is that very few tumor cells are required for this measurement, and it is very likely that sufficient material may be obtained via an FNA. Mutant B-RAF, mutant PTEN, or mutant PIK3CA biomarker status can be determined using analogous techniques.

In the methods of this invention, mutant KRAS, mutant B-RAF, mutant PTEN, or mutant PIK3CA biomarker is preferably assessed by assaying a tumor biopsy. However, in an alternative embodiment, mutant KRAS, B-RAF, PTEN, or PIK3CA biomarker can be assessed in bodily fluids or excretions containing detectable levels of mutant KRAS, B-RAF, PTEN or PIK3CA biomarkers originating from the tumor or tumor cells. Bodily fluids or excretions useful in the present invention include blood, urine, saliva, stool, pleural fluid, lymphatic fluid, sputum, ascites, prostatic fluid, cerebrospinal fluid (CSF), or any other bodily secretion or derivative thereof. By blood it is meant to include whole blood, plasma, serum or any derivative of blood. Assessment of mutant KRAS, B-RAF, PTEN, or PIK3CA in such bodily fluids or excretions can sometimes be preferred in circumstances where an invasive sampling method is inappropriate or inconvenient.

Patient samples or biopsies containing tumor cells can be subjected to a variety of well-known post-collection preparative and storage techniques (e.g., nucleic acid and/or protein extraction, fixation, storage, freezing, ultrafiltration, concentration, evaporation, centrifugation, etc.) prior to assessing the amount of the mutant KRAS, B-RAF, PTEN, or PIK3CA biomarker in the sample. Likewise, tumor biopsies may also be subjected to post-collection preparative and storage techniques, e.g., fixation. Macrodissection and/or microdissection methods (e.g. Laser Microdissection and Pressure Catapulting (LMPC), for example, using the PALM® Micro Beam microscope (P.A.L.M. Microlaser Technologies AG, Bernried, Germany); SL-Microtest UV laser microdissection system (Molecular Machines & Industries, Glattbrugg, Switzerland)) may be used to enrich the tumor cell population of a tumor sample by removing normal tissue cells or stromal cells (e.g. de Bruin E C. et al. BMC Genomics. 2005 Oct. 14; 6:142; Dhal, E. et al. Clinical Cancer Research July 2006 12; 3950; Funel, N. et al. Laboratory Investigation (2008) 88, 773-784, doi:10.1038/labinvest.2008.40, published online 19 May 2008). Primary tumor cell cultures may also be prepared in order to produce a pure tumor cell population.

In the methods of this invention, assessment of KRAS mutation status of tumor cells can be based on any of a number of well-established molecular assays known in the art which have been found to be sufficiently sensitive, specific, and reliable. Many molecular diagnostic laboratories exist to which a sample of a tumor can be sent for KRAS mutation status analysis. The sample can be fresh, frozen or paraffin-embedded tissue depending on the methodology used. Preferably, a pathologist should confirm that a tissue specimen contains cancer cells and estimate the content of tumor cells (percentage tumor nuclei out of all nuclei present) in the specimen. This estimation of tumor cell content is important since different KRAS assays have different analytical sensitivities and an attempt should be made to enrich to a level that is acceptable for the assay being used. For most nucleic acid based assays, the DNA from the tumor sample is extracted for analysis.

Two of the most commonly used methods to evaluate tumor samples for KRAS mutations are real-time PCR and direct sequencing analysis. In real-time PCR, fluorescent probes specific for the most common mutations in codons 12 and 13 are utilized. When a mutation is present, the probe binds and fluorescence is detected. The main requirement for conclusive KRAS genotyping by a PCR assay is the ability to discriminate between different mutant alleles and wild type. In direct sequencing analysis, KRAS mutations are identified using direct sequencing of exon 2 in the KRAS gene. This technique identifies all possible mutations in the exon. Direct sequence analysis has lower analytical sensitivity than some of the real time PCR assays.

A plethora of methods is available for the detection of mutations in the KRAS gene, including for example two KRAS mutation test kits (TheraScreen® by DxS Ltd. (Manchester, UK), and KRAS LightMix® by TIB MolBiol (Berlin, Germany)). An advantage of these commercially available tests is the validation process that these have gone through. Methods available for KRAS genotyping include the following (For a review, see van Krieken J. H. J. M. et al. Virchows Arch (2008) 453:417-431, DOI 10.1007/s00428-008-0665-y):

(A) Gel electrophoresis assays, including temporal temperature gradient electrophoresis [e.g. Kressner U, et al. (1998) Eur J Cancer 34: 518-521], denaturing gradient gel electrophoresis [e.g Hayes V M, et al. (2000) Genes Chromosomes Cancer 29: 309-314], constant denaturant capillary electrophoresis [e.g Zhao C, et al. (2004) Biomed Chromatogr 18: 538-541], and SSCP (single-strand conformation polymorphism) assay [e.g Chaubert P, et al. (1993). Biotechniques 15: 586];

(B) Sequencing methods, including dideoxy sequencing [e.g Khanna M, et al. (1999) Oncogene 18: 27-38], pyrosequencing [e.g Ogino S, et al. (2005) J Mol Diagn 7: 413-421; Poehlmann A, et al. (2007) Pathol Res Pract 203: 489-497], PyroMark™ KRAS assays;

(C) Allele-specific PCR assays, including those based on (i) Allele discrimination based on primer design, e.g. ARMS-PCR [e.g Fox J C, et al. (1998) Br J Cancer 77: 1267-1274; van Heek N T, et al. (2005) J Clin Pathol 58: 1315-1320], a TheraScreen® kit [e.g Cross J. (2008) DxS Ltd. Pharmacogenomics 9: 463-467], a KRAS LightMix® kit, REMS-PCR [e.g Mixich F, et al. (2007) J Gastrointestin Liver Dis 16: 5-10], a FLAG assay [e.g Amicarelli G, et al. (2007) Nucleic Acids Res 35: e131], enriched PCR-RFLP [e.g Kimura K, et al. (2007) J Int Med Res 35: 450-457]; (ii) Allele discrimination based on allele-specific ligation detection reaction, e.g. PCR-LDR [e.g Hashimoto M, et al. (2007) Analyst 132: 913-921], and PCR-LDR spFRET (single-pair fluorescence resonance energy transfer) assay [e.g Wabuyele M B, et al. (2003) J Am Chem Soc 125: 6937-6945]; and (iii) Allele discrimination based on discriminating amplification efficiencies at low melting temperatures, e.g. COLD-PCR [e.g Li J, et al. (2007) Anal Chem 79: 9030-9038].

Other methods for mutant K-RAS determination include surface ligation reaction and biometallization [e.g Zhang P, et al. (2008) Biosens Bioelectron 23: 1435-1441]; multi-target DNA assay panel [e.g Syngal S, et al. (2006) Cancer 106: 277-283]; and allele-specific oligonucleotide hybridization (Invigene®).

Further methods for mutant K-RAS determination are also disclosed in Krypuy M, et al. High resolution melting analysis for the rapid and sensitive detection of mutations in clinical samples: KRAS codon 12 and 13 mutations in non-small cell lung cancer. BMC Cancer 2006, 6: 295 doi: 10.1186/1471-2407-6-295. www.biomedcentral.com/1471-2407/6/295/; and in Ogino S, et al. Sensitive Sequencing Method for KRAS Mutation Detection by Pyrosequencing. J Mol Diagn 7: 413-421, 2005. http//jmd.amjpathol.org/cgi/content/abstract/7/3/413.

Many of the methods described herein for detection of mutations in the KRAS gene can be readily applied for detection of mutations in other genes, including the B-RAF gene, the PTEN gene, or the PIK3CA gene.

Specific methods and kits available for the detection of mutations in the B-RAF gene, include the following: (a) A B-RAF Mutation Test Kit that can detect the V600E mutation in tumor cell samples, the most common B-RAF mutation (DxS Ltd., Manchester, UK, now part of QIAGEN N.V., Frankfurt, Germany), based on a combination of ARMS® (allele specific PCR) with Scorpions® real-time PCR technology used on tumor cell extracted DNA; (b) a BFAF gene mutation (V600E) assay (EntroGen, Tarzana, Calif.), as part of a KRAS/BRAF mutation panel, using allele-specific PCR methodology; (c) A shifted-termination PCR assay for enriching mutation signals, with mutation detection by fragment analysis, available for V600E (T1799A), V600G (T1799G), and V600A (T1799C) mutations (Trimgen Genetic Diagnostics, Sparks, Md.); and (d) a BRAF Pyrosequencing Assay for Mutation Detection (Spittle, C et al, (2007) Journal of Molecular Diagnostics 2007, Vol. 9, No. 4, 464-471; DOI: 10.2353/jmoldx.2007.060191), that can readily detect the common V600E mutation, and additional mutations affecting codons 600 or 601 (e.g. V600K, V600D, V600R, and K601E).

Specific methods and kits available for the detection of mutations in the PIK3CA gene, include the following: (a) A PIK3CA Mutation Test Kit that can detect the E542K (G1624A), E545K (G1633A), E545D (G1635T), and H1047R (A3140G) mutations in tumor cell samples (DxS Ltd., Manchester, UK, now part of QIAGEN N.V., Frankfurt, Germany), based on a combination of ARMS® (allele specific PCR) with Scorpions® real-time PCR technology used on tumor cell extracted DNA; and (b) A shifted-termination PCR assay for enriching mutation signals, with mutation detection by fragment analysis, available for E542K (G1624A), E545K (G1633A), E545G (A1634C), H1047R (A3140G), H1047L (A3140T) mutations (Trimgen Genetic Diagnostics, Sparks, Md.).

In an alternative embodiment, mutant KRAS, B-RAF, PTEN, or PIK3CA protein is assessed using an antibody (e.g. a radio-labeled, chromophore-labeled, fluorophore-labeled, or enzyme-labeled antibody), an antibody derivative (e.g. an antibody conjugated with a substrate or with the protein or ligand of a protein-ligand pair (e.g. biotin-streptavidin)), or an antibody fragment (e.g. a single-chain antibody, an isolated antibody hypervariable domain, etc.) which binds specifically to mutant KRAS, BRAF, PTEN, or PIK3CA protein.

In another embodiment of the present invention, mutant KRAS, B-RAF, PTEN, or PIK3CA biomarker protein is detected. A preferred agent for detecting biomarker protein of the invention is an antibody capable of specific binding to mutant KRAS, B-RAF, PTEN, or PIK3CA protein, or a fragment thereof, preferably an antibody with a detectable label. Antibodies can be polyclonal, or more preferably, monoclonal. An intact antibody, or a fragment or derivative thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

Proteins from tumor cells can be isolated using techniques that are well known to those of skill in the art. The protein isolation methods employed can, for example, be such as those described in Harlow and Lane (Harlow and Lane, 1988, Antibodies: A Laboratory Manual, Cold Spring Harbor Laboratory Press, Cold Spring Harbor, N.Y.).

A variety of formats can be employed to determine whether a sample contains a protein that binds to a given antibody. Examples of such formats include, but are not limited to, enzyme immunoassay (EIA), radioimmunoassay (RIA), Western blot analysis and enzyme linked immunoabsorbant assay (ELISA). A skilled artisan can readily adapt known protein/antibody detection methods for use in determining whether tumor cells express a mutant protein biomarker of the present invention.

In one format, antibodies, or antibody fragments or derivatives, can be used in methods such as Western blots or immunofluorescence techniques to detect the expressed proteins. In such uses, it is generally preferable to immobilize either the antibody or proteins on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present invention. For example, protein isolated from tumor cells can be run on a polyacrylamide gel electrophoresis and immobilized onto a solid phase support such as nitrocellulose. The support can then be washed with suitable buffers followed by treatment with the detectably labeled antibody. The solid phase support can then be washed with the buffer a second time to remove unbound antibody. The amount of bound label on the solid support can then be detected by conventional means.

For ELISA assays, specific binding pairs can be of the immune or non-immune type. Immune specific binding pairs are exemplified by antigen-antibody systems or hapten/anti-hapten systems. There can be mentioned fluorescein/anti-fluorescein, dinitrophenyl/anti-dinitrophenyl, biotin/anti-biotin, peptide/anti-peptide and the like. The antibody member of the specific binding pair can be produced by customary methods familiar to those skilled in the art. Such methods involve immunizing an animal with the antigen member of the specific binding pair. If the antigen member of the specific binding pair is not immunogenic, e.g., a hapten, it can be covalently coupled to a carrier protein to render it immunogenic. Non-immune binding pairs include systems wherein the two components share a natural affinity for each other but are not antibodies. Exemplary non-immune pairs are biotin-streptavidin, intrinsic factor-vitamin B₁₂, folic acid-folate binding protein and the like.

A variety of methods are available to covalently label antibodies with members of specific binding pairs. Methods are selected based upon the nature of the member of the specific binding pair, the type of linkage desired, and the tolerance of the antibody to various conjugation chemistries. Biotin can be covalently coupled to antibodies by utilizing commercially available active derivatives. Some of these are biotin-N-hydroxy-succinimide which binds to amine groups on proteins; biotin hydrazide which binds to carbohydrate moieties, aldehydes and carboxyl groups via a carbodiimide coupling; and biotin maleimide and iodoacetyl biotin which bind to sulfhydryl groups. Fluorescein can be coupled to protein amine groups using fluorescein isothiocyanate. Dinitrophenyl groups can be coupled to protein amine groups using 2,4-dinitrobenzene sulfate or 2,4-dinitrofluorobenzene. Other standard methods of conjugation can be employed to couple monoclonal antibodies to a member of a specific binding pair including dialdehyde, carbodiimide coupling, homofunctional crosslinking, and heterobifunctional crosslinking. Carbodiimide coupling is an effective method of coupling carboxyl groups on one substance to amine groups on another. Carbodiimide coupling is facilitated by using the commercially available reagent 1-ethyl-3-(dimethyl-aminopropyl)-carbodiimide (EDAC).

Homobifunctional crosslinkers, including the bifunctional imidoesters and bifunctional N-hydroxysuccinimide esters, are commercially available and are employed for coupling amine groups on one substance to amine groups on another. Heterobifunctional crosslinkers are reagents which possess different functional groups. The most common commercially available heterobifunctional crosslinkers have an amine reactive N-hydroxysuccinimide ester as one functional group, and a sulfhydryl reactive group as the second functional group. The most common sulfhydryl reactive groups are maleimides, pyridyl disulfides and active halogens. One of the functional groups can be a photoactive aryl nitrene, which upon irradiation reacts with a variety of groups.

The detectably-labeled antibody or detectably-labeled member of the specific binding pair is prepared by coupling to a reporter, which can be a radioactive isotope, enzyme, fluorogenic, chemiluminescent or electrochemical materials. Two commonly used radioactive isotopes are ¹²⁵I and ³H. Standard radioactive isotopic labeling procedures include the chloramine T, lactoperoxidase and Bolton-Hunter methods for ¹²⁵I and reductive methylation for ³H. The term “detectably-labeled” refers to a molecule labeled in such a way that it can be readily detected by the intrinsic enzymic activity of the label or by the binding to the label of another component, which can itself be readily detected.

Enzymes suitable for use in this invention include, but are not limited to, horseradish peroxidase, alkaline phosphatase, β-galactosidase, glucose oxidase, luciferases, including firefly and renilla, β-lactamase, urease, green fluorescent protein (GFP) and lysozyme. Enzyme labeling is facilitated by using dialdehyde, carbodiimide coupling, homobifunctional crosslinkers and heterobifunctional crosslinkers as described above for coupling an antibody with a member of a specific binding pair.

The labeling method chosen depends on the functional groups available on the enzyme and the material to be labeled, and the tolerance of both to the conjugation conditions. The labeling method used in the present invention can be one of, but not limited to, any conventional methods currently employed including those described by Engvall and Pearlmann, Immunochemistry 8, 871 (1971), Avrameas and Ternynck, Immunochemistry 8, 1175 (1975), Ishikawa et al., J. Immunoassay 4(3):209-327 (1983) and Jablonski, Anal. Biochem. 148:199 (1985).

Labeling can be accomplished by indirect methods such as using spacers or other members of specific binding pairs. An example of this is the detection of a biotinylated antibody with unlabeled streptavidin and biotinylated enzyme, with streptavidin and biotinylated enzyme being added either sequentially or simultaneously. Thus, according to the present invention, the antibody used to detect can be detectably-labeled directly with a reporter or indirectly with a first member of a specific binding pair. When the antibody is coupled to a first member of a specific binding pair, then detection is effected by reacting the antibody-first member of a specific binding complex with the second member of the binding pair that is labeled or unlabeled as mentioned above.

Moreover, the unlabeled detector antibody can be detected by reacting the unlabeled antibody with a labeled antibody specific for the unlabeled antibody. In this instance “detectably-labeled” as used above is taken to mean containing an epitope by which an antibody specific for the unlabeled antibody can bind. Such an anti-antibody can be labeled directly or indirectly using any of the approaches discussed above. For example, the anti-antibody can be coupled to biotin which is detected by reacting with the streptavidin-horseradish peroxidase system discussed above.

In one embodiment of this invention biotin is utilized. The biotinylated antibody is in turn reacted with streptavidin-horseradish peroxidase complex. Orthophenylenediamine, 4-chloro-naphthol, tetramethylbenzidine (TMB), ABTS, BTS or ASA can be used to effect chromogenic detection.

In one immunoassay format for practicing this invention, a forward sandwich assay is used in which the capture reagent has been immobilized, using conventional techniques, on the surface of a support. Suitable supports used in assays include synthetic polymer supports, such as polypropylene, polystyrene, substituted polystyrene, e.g. aminated or carboxylated polystyrene, polyacrylamides, polyamides, polyvinylchloride, glass beads, agarose, or nitrocellulose.

The invention also encompasses kits for detecting the presence of a mutant KRAS, BRAF or PIK3CA protein or nucleic acid in a biological sample. Such kits can be used to determine whether a subject is suffering from a tumor that is either susceptible or resistant to inhibition by IGF-1R kinase inhibitors. For example, the kit can comprise a labeled compound or agent capable of detecting a mutant protein or nucleic acid in a biological sample and means for determining the amount of the protein or mRNA in the sample (e.g., an antibody which binds the protein or a fragment thereof, or an oligonucleotide probe which binds to DNA or mRNA encoding the protein). Kits can also include instructions for interpreting the results obtained using the kit.

For antibody-based kits, the kit can comprise, for example: (1) a first antibody (e.g., attached to a solid support) which binds to a biomarker protein; and, optionally, (2) a second, different antibody which binds to either the protein or the first antibody and is conjugated to a detectable label.

The present invention further provides any of the methods described herein for treating tumors or tumor metastases, or cancer, in a patient comprising administering to the patient a therapeutically effective amount of an IGF-1R kinase inhibitor, and in addition, simultaneously or sequentially, one or more other cytotoxic, chemotherapeutic or anti-cancer agents, or compounds that enhance the effects of such agents. In the context of this invention, other anti-cancer agents includes, for example, other cytotoxic, chemotherapeutic or anti-cancer agents, or compounds that enhance the effects of such agents, anti-hormonal agents, angiogenesis inhibitors, agents that inhibit or reverse EMT (e.g. TGF-beta receptor inhibitors), tumor cell pro-apoptotic or apoptosis-stimulating agents, histone deacetylase (HDAC) inhibitors, histone demethylase inhibitors, DNA methyltransferase inhibitors, signal transduction inhibitors, anti-proliferative agents, anti-HER2 antibody or an immunotherapeutically active fragment thereof, anti-proliferative agents, COX II (cyclooxygenase II) inhibitors, and agents capable of enhancing antitumor immune responses, as described herein.

In the context of this invention, additional other cytotoxic, chemotherapeutic or anti-cancer agents, or compounds that enhance the effects of such agents, include, for example: alkylating agents or agents with an alkylating action, such as cyclophosphamide (CTX; e.g. CYTOXAN®), chlorambucil (CHL; e.g. LEUKERAN®), cisplatin (CisP; e.g. PLATINOL®) busulfan (e.g. MYLERAN®), melphalan, carmustine (BCNU), streptozotocin, triethylenemelamine (TEM), mitomycin C, and the like; anti-metabolites, such as methotrexate (MTX), etoposide (VP16; e.g. VEPESID®), 6-mercaptopurine (6MP), 6-thiocguanine (6TG), cytarabine (Ara-C), 5-fluorouracil (5-FU), capecitabine (e.g. XELODA®), dacarbazine (DTIC), and the like; antibiotics, such as actinomycin D, doxorubicin (DXR; e.g. ADRIAMYCIN®), daunorubicin (daunomycin), bleomycin, mithramycin and the like; alkaloids, such as vinca alkaloids such as vincristine (VCR), vinblastine, and the like; and other antitumor agents, such as paclitaxel (e.g. TAXOL®) and pactitaxel derivatives, the cytostatic agents, glucocorticoids such as dexamethasone (DEX; e.g. DECADRON®) and corticosteroids such as prednisone, nucleoside enzyme inhibitors such as hydroxyurea, amino acid depleting enzymes such as asparaginase, leucovorin and other folic acid derivatives, and similar, diverse antitumor agents. The following agents may also be used as additional agents: arnifostine (e.g. ETHYOL®), dactinomycin, mechlorethamine (nitrogen mustard), streptozocin, cyclophosphamide, lomustine (CCNU), doxorubicin lipo (e.g. DOXIL®), gemcitabine (e.g. GEMZAR®), daunorubicin lipo (e.g. DAUNOXOME®), procarbazine, mitomycin, docetaxel (e.g. TAXOTERE®), aldesleukin, carboplatin, oxaliplatin, cladribine, camptothecin, CPT 11 (irinotecan), 10-hydroxy 7-ethyl-camptothecin (SN38), floxuridine, fludarabine, ifosfamide, idarubicin, mesna, interferon beta, interferon alpha, mitoxantrone, topotecan, leuprolide, megestrol, melphalan, mercaptopurine, plicamycin, mitotane, pegaspargase, pentostatin, pipobroman, plicamycin, tamoxifen, teniposide, testolactone, thioguanine, thiotepa, uracil mustard, vinorelbine, chlorambucil and pemetrexed.

The present invention further provides any of the methods described herein for treating cancer, or tumors or tumor metastases, in a patient comprising administering to the patient a therapeutically effective amount of an IGF-1R kinase inhibitor, and in addition, simultaneously or sequentially, one or more anti-hormonal agents. As used herein, the term “anti-hormonal agent” includes natural or synthetic organic or peptidic compounds that act to regulate or inhibit hormone action on tumors.

Antihormonal agents include, for example: steroid receptor antagonists, anti-estrogens such as tamoxifen, raloxifene, aromatase inhibiting 4(5)-imidazoles, other aromatase inhibitors, exemestane, anastrozole, letrozole, vorozole, formestane, fadrozole, aminoglutethimide, testolactone, 42-hydroxytamoxifen, trioxifene, keoxifene, LY 117018, onapristone, and toremifene (e.g. FARESTON®); anti-androgens such as flutamide, nilutamide, bicalutamide, leuprolide, and goserelin; and pharmaceutically acceptable salts, acids or derivatives of any of the above; agonists and/or antagonists of glycoprotein hormones such as follicle stimulating hormone (FSH), thyroid stimulating hormone (TSH), and luteinizing hormone (LH) and LHRH (leuteinizing hormone-releasing hormone); the LHRH agonist goserelin acetate, commercially available as ZOLADEX® (AstraZeneca); the LHRH antagonist D-alaninamide N-acetyl-3-(2-naphthalenyl)-D-alanyl-4-chloro-D-phenylalanyl-3-(3-pyridinyl)-D-alanyl-L-seryl-N6-(3-pyridinylcarbonyl)-L-lysyl-N6-(3-pyridinylcarbonyl)-D-lysyl-L-leucyl-N6-(1-methylethyl)-L-lysyl-L-proline (e.g ANTIDE®, Ares-Serono); the LHRH antagonist ganirelix acetate; the steroidal anti-androgens cyproterone acetate (CPA) and megestrol acetate, commercially available as MEGACE® (Bristol-Myers Oncology); the nonsteroidal anti-androgen flutamide (2-methyl-N-[4,20-nitro-3-(trifluoromethyl)phenylpropanamide), commercially available as EULEXIN® (Schering Corp.); the non-steroidal anti-androgen nilutamide, (5,5-dimethyl-3-[4-nitro-3-(trifluoromethyl-4′-nitrophenyl)-4,4-dimethyl-imidazolidine-dione); and antagonists for other non-permissive receptors, such as antagonists for RAR, RXR, TR, VDR, and the like.

The use of the cytotoxic and other anticancer agents described above in chemotherapeutic regimens is generally well characterized in the cancer therapy arts, and their use herein falls under the same considerations for monitoring tolerance and effectiveness and for controlling administration routes and dosages, with some adjustments. For example, the actual dosages of the cytotoxic agents may vary depending upon the patient's cultured cell response determined by using histoculture methods. Generally, the dosage will be reduced compared to the amount used in the absence of additional other agents.

Typical dosages of an effective cytotoxic agent can be in the ranges recommended by the manufacturer, and where indicated by in vitro responses or responses in animal models, can be reduced by up to about one order of magnitude concentration or amount. Thus, the actual dosage will depend upon the judgment of the physician, the condition of the patient, and the effectiveness of the therapeutic method based on the in vitro responsiveness of the primary cultured malignant cells or histocultured tissue sample, or the responses observed in the appropriate animal models.

The present invention further provides any of the methods described herein for treating tumors or tumor metastases in a patient comprising administering to the patient a therapeutically effective amount of an IGF-1R kinase inhibitor, and in addition, simultaneously or sequentially, one or more angiogenesis inhibitors.

Anti-angiogenic agents include, for example: VEGFR inhibitors, such as SU-5416 and SU-6668 (Sugen Inc. of South San Francisco, Calif., USA), or as described in, for example International Application Nos. WO 99/24440, WO 99/62890, WO 95/21613, WO 99/61422, WO 98/50356, WO 99/10349, WO 97/32856, WO 97/22596, WO 98/54093, WO 98/02438, WO 99/16755, and WO 98/02437, and U.S. Pat. Nos. 5,883,113, 5,886,020, 5,792,783, 5,834,504 and 6,235,764; VEGF inhibitors such as IM862 (Cytran Inc. of Kirkland, Wash., USA); sunitinib (Pfizer); angiozyme, a synthetic ribozyme from Ribozyme (Boulder, Colo.) and Chiron (Emeryville, Calif.); and antibodies to VEGF, such as bevacizumab (e.g. AVASTIN™, Genentech, South San Francisco, Calif.), a recombinant humanized antibody to VEGF; integrin receptor antagonists and integrin antagonists, such as to αᵥβ₃, αᵥβ₅ and αᵥβ₆ integrins, and subtypes thereof, e.g. cilengitide (EMD 121974), or the anti-integrin antibodies, such as for example αᵥβ₃ specific humanized antibodies (e.g. VITAXIN®); factors such as IFN-alpha (U.S. Pat. Nos. 41530,901, 4,503,035, and 5,231,176); angiostatin and plasminogen fragments (e.g. kringle 1-4, kringle 5, kringle 1-3 (O'Reilly, M. S. et al. (1994) Cell 79:315-328; Cao et al. (1996) J. Biol. Chem. 271: 29461-29467; Cao et al. (1997) J. Biol. Chem. 272:22924-22928); endostatin (O'Reilly, M. S. et al. (1997) Cell 88:277; and International Patent Publication No. WO 97/15666); thrombospondin (TSP-1; Frazier, (1991) Curr. Opin. Cell Biol. 3:792); platelet factor 4 (PF4); plasminogen activator/urokinase inhibitors; urokinase receptor antagonists; heparinases; fumagillin analogs such as TNP-4701; suramin and suramin analogs; angiostatic steroids; bFGF antagonists; flk-1 and flt-1 antagonists; anti-angiogenesis agents such as MMP-2 (matrix-metalloproteinase 2) inhibitors and MMP-9 (matrix-metalloproteinase 9) inhibitors. Examples of useful matrix metalloproteinase inhibitors are described in International Patent Publication Nos. WO 96/33172, WO 96/27583, WO 98/07697, WO 98/03516, WO 98/34918, WO 98/34915, WO 98/33768, WO 98/30566, WO 90/05719, WO 99/52910, WO 99/52889, WO 99/29667, and WO 99/07675, European Patent Publication Nos. 818,442, 780,386, 1,004,578, 606,046, and 931,788; Great Britain Patent Publication No. 9912961, and U.S. Pat. Nos. 5,863,949 and 5,861,510. Preferred MMP-2 and MMP-9 inhibitors are those that have little or no activity inhibiting MMP-1. More preferred, are those that selectively inhibit MMP-2 and/or MMP-9 relative to the other matrix-metalloproteinases (i.e. MMP-1, MMP-3, MMP-4, MMP-5, MMP-6, MMP-7, MMP-8, MMP-10, MMP-11, MMP-12, and MMP-13).

The present invention further provides any of the methods described herein for treating cancer, or tumors or tumor metastases, in a patient comprising administering to the patient a therapeutically effective amount of an IGF-1R kinase inhibitor, and in addition, simultaneously or sequentially, one or more tumor cell pro-apoptotic or apoptosis-stimulating agents.

The present invention further provides any of the methods described herein for treating cancer, or tumors or tumor metastases, in a patient comprising administering to the patient a therapeutically effective amount of an IGF-1R kinase inhibitor, and in addition, simultaneously or sequentially, one or more signal transduction inhibitors.

Signal transduction inhibitors include, for example: erbB2 receptor inhibitors, such as organic molecules, or antibodies that bind to the erbB2 receptor, for example, trastuzumab (e.g. HERCEPTIN®); inhibitors of other protein tyrosine-kinases, e.g. imitinib (e.g. GLEEVEC®); EGFR kinase inhibitors (see herein below); Met kinase inhibitors (e.g. PF-2341066); ras inhibitors; raf inhibitors; MEK inhibitors; mTOR inhibitors, including mTOR inhibitors that bind to and directly inhibits both mTORC1 and mTORC2 kinases (e.g. OSI-027, OSI Pharmaceuticals); mTOR inhibitors that are dual PI3K/mTOR kinase inhibitors, such as for example the compound PI-103 as described in Fan, Q-W et al (2006) Cancer Cell 9:341-349 and Knight, Z. A. et al. (2006) Cell 125:733-747; mTOR inhibitors that are dual inhibitors of mTOR kinase and one or more other PIKK (or PIK-related) kinase family members. Such members include MEC1, TEL1, RAD3, MEI-41, DNA-PK, ATM, ATR, TRRAP, PI3K, and PI4K kinases; cyclin dependent kinase inhibitors; protein kinase C inhibitors; PI-3 kinase inhibitors; and PDK-1 inhibitors (see Dancey, J. and Sausville, E. A. (2003) Nature Rev. Drug Discovery 2:92-313, for a description of several examples of such inhibitors, and their use in clinical trials for the treatment of cancer).

EGFR kinase inhibitors include, for example: [6,7-bis(2-methoxyethoxy)-4-quinazolin-4-yl]-(3-ethynylphenyl)amine (also known as OSI-774, erlotinib, or TARCEVA™ (erlotinib HCl); OSI Pharmaceuticals/Genentech/Roche) (U.S. Pat. No. 5,747,498; International Patent Publication No. WO 01/34574, and Moyer, J. D. et al. (1997) Cancer Res. 57:4838-4848); CI-1033 (formerly known as PD183805; Pfizer) (Sherwood et al., 1999, Proc. Am. Assoc. Cancer Res. 40:723); PD-158780 (Pfizer); AG-1478 (University of California); CGP-59326 (Novartis); PKI-166 (Novartis); EKB-569 (Wyeth); GW-2016 (also known as GW-572016 or lapatinib ditosylate; GSK); gefitinib (also known as ZD1839 or IRESSA™; Astrazeneca) (Woodburn et al., 1997, Proc. Am. Assoc. Cancer Res. 38:633); and antibody-based EGFR kinase inhibitors. A particularly preferred low molecular weight EGFR kinase inhibitor that can be used according to the present invention is [6,7-bis(2-methoxyethoxy)-4-quinazolin-4-yl]-(3-ethynylphenyl)amine (i.e. erlotinib), its hydrochloride salt (i.e. erlotinib HCl, TARCEVA™), or other salt forms (e.g. erlotinib mesylate). Antibody-based EGFR kinase inhibitors include any anti-EGFR antibody or antibody fragment that can partially or completely block EGFR activation by its natural ligand. Non-limiting examples of antibody-based EGFR kinase inhibitors include those described in Modjtahedi, H., et al., 1993, Br. J. Cancer 67:247-253; Teramoto, T., et al., 1996, Cancer 77:639-645; Goldstein et al., 1995, Clin. Cancer Res. 1:1311-1318; Huang, S. M., et al., 1999, Cancer Res. 15:59(8):1935-40; and Yang, X., et al., 1999, Cancer Res. 59:1236-1243. Thus, the EGFR kinase inhibitor can be the monoclonal antibody Mab E7.6.3 (Yang, X. D. et al. (1999) Cancer Res. 59:1236-43), or Mab C225 (ATCC Accession No. HB-8508), or an antibody or antibody fragment having the binding specificity thereof. Suitable monoclonal antibody EGFR kinase inhibitors include, but are not limited to, IMC-C225 (also known as cetuximab or ERBITUX™; Imclone Systems), ABX-EGF (Abgenix), EMD 72000 (Merck KgaA, Darmstadt), RH3 (York Medical Bioscience Inc.), and MDX-447 (Medarex/Merck KgaA).

EGFR kinase inhibitors also include, for example multi-kinase inhibitors that have activity on EGFR kinase, i.e. inhibitors that inhibit EGFR kinase and one or more additional kinases. Examples of such compounds include the EGFR and HER2 inhibitor CI-1033 (formerly known as PD183805; Pfizer); the EGFR and HER2 inhibitor GW-2016 (also known as GW-572016 or lapatinib ditosylate; GSK); the EGFR and JAK 2/3 inhibitor AG490 (a tyrphostin); the EGFR and HER2 inhibitor ARRY-334543 (Array BioPharma); BIBW-2992, an irreversible dual EGFR/HER2 kinase inhibitor (Boehringer Ingelheim Corp.); the EGFR and HER2 inhibitor EKB-569 (Wyeth); the VEGF-R2 and EGFR inhibitor ZD6474 (also known as ZACTIMA™; AstraZeneca Pharmaceuticals), and the EGFR and HER2 inhibitor BMS-599626 (Bristol-Myers Squibb).

ErbB2 receptor inhibitors include, for example: ErbB2 receptor inhibitors, such as lapatinib or GW-282974 (both Glaxo Wellcome plc), monoclonal antibodies such as AR-209 (Aronex Pharmaceuticals Inc. of The Woodlands, Tex., USA) and 2B-1 (Chiron), and erbB2 inhibitors such as those described in International Publication Nos. WO 98/02434, WO 99/35146, WO 99/35132, WO 98/02437, WO 97/13760, and WO 95/19970, and U.S. Pat. Nos. 5,587,458, 5,877,305, 6,465,449 and 6,541,481.

The present invention further provides any of the methods described herein for treating cancer, or tumors or tumor metastases, in a patient comprising administering to the patient a therapeutically effective amount of an IGF-1R kinase inhibitor, and in addition, simultaneously or sequentially, an anti-HER2 antibody (e.g. trastuzumab, Genentech) or an immunotherapeutically active fragment thereof.

The present invention further provides any of the methods described herein for treating cancer, or tumors or tumor metastases, in a patient comprising administering to the patient a therapeutically effective amount of an IGF-1R kinase inhibitor, and in addition, simultaneously or sequentially, one or more additional anti-proliferative agents.

Additional antiproliferative agents include, for example: Inhibitors of the enzyme farnesyl protein transferase and inhibitors of the receptor tyrosine kinase PDGFR, including the compounds disclosed and claimed in U.S. Pat. Nos. 6,080,769, 6,194,438, 6,258,824, 6,586,447, 6,071,935, 6,495,564, 6,150,377, 6,596,735 and 6,479,513, and International Patent Publication WO 01/40217, and FGFR kinase inhibitors.

Examples of PDGFR kinase inhibitors that can be used according to the present invention include Imatinib (GLEEVEC®; Novartis); SU-12248 (sunitinib malate, SUTENT®; Pfizer); Dasatinib (SPRYCEL®; BMS; also known as BMS-354825); Sorafenib (NEXAVAR®; Bayer; also known as Bay-43-9006); AG-13736 (Axitinib; Pfizer); RPR127963 (Sanofi-Aventis); CP-868596 (Pfizer/OSI Pharmaceuticals); MLN-518 (tandutinib; Millennium Pharmaceuticals); AMG-706 (Motesanib; Amgen); ARAVA® (leflunomide; Sanofi-Aventis; also known as SU101), and OSI-930 (OSI Pharmaceuticals); Additional preferred examples of low molecular weight PDGFR kinase inhibitors that are also FGFR kinase inhibitors that can be used according to the present invention include XL-999 (Exelixis); SU6668 (Pfizer); CHIR-258/TKI-258 (Chiron); RO4383596 (Hoffmann-La Roche) and BIBF-1120 (Boehringer Ingelheim).

Examples of FGFR kinase inhibitors that can be used according to the present invention include RO-4396686 (Hoffmann-La Roche); CHIR-258 (Chiron; also known as TKI-258); PD 173074 (Pfizer); PD 166866 (Pfizer); ENK-834 and ENK-835 (both Enkam Pharmaceuticals A/S); and SU5402 (Pfizer). Additional preferred examples of low molecular weight FGFR kinase inhibitors that are also PDGFR kinase inhibitors that can be used according to the present invention include XL-999 (Exelixis); SU6668 (Pfizer); CHIR-258/TKI-258 (Chiron); RO4383596 (Hoffmann-La Roche), and BIBF-1120 (Boehringer Ingelheim).

The present invention further provides any of the methods described herein for treating cancer, or tumors or tumor metastases, in a patient comprising administering to the patient a therapeutically effective amount of an IGF-1R kinase inhibitor, and in addition, simultaneously or sequentially, a COX II (cyclooxygenase II) inhibitor. Examples of useful COX-II inhibitors include alecoxib (e.g. CELEBREX™), valdecoxib, and rofecoxib.

The present invention further provides any of the methods described herein for treating cancer, or tumors or tumor metastases, in a patient comprising administering to the patient a therapeutically effective amount of an IGF-1R kinase inhibitor, and in addition, simultaneously or sequentially, treatment with radiation or a radiopharmaceutical.

The source of radiation can be either external or internal to the patient being treated. When the source is external to the patient, the therapy is known as external beam radiation therapy (EBRT). When the source of radiation is internal to the patient, the treatment is called brachytherapy (BT). Radioactive atoms for use in the context of this invention can be selected from the group including, but not limited to, radium, cesium-137, iridium-192, americium-241, gold-198, cobalt-57, copper-67, technetium-99, iodine-123, iodine-131, and indium-111. Where the IGF-1R kinase inhibitor according to this invention is an antibody, it is also possible to label the antibody with such radioactive isotopes.

Radiation therapy is a standard treatment for controlling unresectable or inoperable tumors and/or tumor metastases. Improved results have been seen when radiation therapy has been combined with chemotherapy. Radiation therapy is based on the principle that high-dose radiation delivered to a target area will result in the death of reproductive cells in both tumor and normal tissues. The radiation dosage regimen is generally defined in terms of radiation absorbed dose (Gy), time and fractionation, and must be carefully defined by the oncologist. The amount of radiation a patient receives will depend on various considerations, but the two most important are the location of the tumor in relation to other critical structures or organs of the body, and the extent to which the tumor has spread. A typical course of treatment for a patient undergoing radiation therapy will be a treatment schedule over a 1 to 6 week period, with a total dose of between 10 and 80 Gy administered to the patient in a single daily fraction of about 1.8 to 2.0 Gy, 5 days a week. In a preferred embodiment of this invention there is synergy when tumors in human patients are treated with the combination treatment of the invention and radiation. In other words, the inhibition of tumor growth by means of the agents comprising the combination of the invention is enhanced when combined with radiation, optionally with additional chemotherapeutic or anticancer agents. Parameters of adjuvant radiation therapies are, for example, contained in International Patent Publication WO 99/60023.

The present invention further provides any of the methods described herein for treating cancer, or tumors or tumor metastases, in a patient comprising administering to the patient a therapeutically effective amount of an IGF-1R kinase inhibitor, and in addition, simultaneously or sequentially, treatment with one or more agents capable of enhancing antitumor immune responses.

Agents capable of enhancing antitumor immune responses include, for example: CTLA4 (cytotoxic lymphocyte antigen 4) antibodies (e.g. MDX-CTLA4, ipilimumab, MDX-010), and other agents capable of blocking CTLA4. Specific CTLA4 antibodies that can be used in the present invention include those described in U.S. Pat. No. 6,682,736.

In the context of this invention, an “effective amount” of an agent or therapy is as defined above. A “sub-therapeutic amount” of an agent or therapy is an amount less than the effective amount for that agent or therapy, but when combined with an effective or sub-therapeutic amount of another agent or therapy can produce a result desired by the physician, due to, for example, synergy in the resulting efficacious effects, or reduced side effects.

As used herein, the term “patient” preferably refers to a human in need of treatment with an IGF-1R kinase inhibitor for cancer, including refractory versions of such cancers that have failed to respond to other treatments. The cancers, or tumors and tumor metastases, of this invention include NSCL (non-small cell lung), pancreatic, head and neck, oral or nasal squamous cell carcinoma, colon, ovarian or breast cancers, lung cancer, bronchioloalveolar cell lung cancer, bone cancer, skin cancer, cancer of the head or neck, HNSCC, cutaneous or intraocular melanoma, uterine cancer, ovarian cancer, rectal cancer, cancer of the anal region, stomach cancer, gastric cancer, uterine cancer, carcinoma of the fallopian tubes, carcinoma of the endometrium, carcinoma of the vagina, carcinoma of the vulva, Hodgkin's Disease, cancer of the esophagus, cancer of the small intestine, colorectal cancer, cancer of the endocrine system, cancer of the thyroid gland, cancer of the parathyroid gland, cancer of the adrenal gland, adrenocortical carcinoma (ACC), sarcoma of soft tissue, Ewing's sarcoma, rhabdomyosarcoma, myeloma, multiple myeloma, cancer of the urethra, cancer of the penis, prostate cancer, cancer of the bladder, cancer of the ureter, carcinoma of the renal pelvis, mesothelioma, hepatocellular cancer, biliary cancer, cancer of the kidney, renal cell carcinoma, chronic or acute leukemia, lymphocytic lymphomas, neuroblastoma, neoplasms of the central nervous system (CNS), spinal axis tumors, brain stem glioma, glioblastoma multiforme, astrocytomas, schwannomas, ependymomas, medulloblastomas, meningiomas, squamous cell carcinomas, pituitary adenomas, including refractory versions of any of the above cancers, or a combination of one or more of the above cancers. In addition to cancer, the methods of this invention may also be used for precancerous conditions or lesions, including, for example, oral leukoplakia, actinic keratosis (solar keratosis), precancerous polyps of the colon or rectum, gastric epithelial dysplasia, adenomatous dysplasia, hereditary nonpolyposis colon cancer syndrome (HNPCC), Barrett's esophagus, bladder dysplasia, liver cirrhosis or scarring, and precancerous cervical conditions.

The term “refractory” as used herein is used to define a cancer for which treatment (e.g. chemotherapy drugs, biological agents, and/or radiation therapy) has proven to be ineffective. A refractory cancer tumor may shrink, but not to the point where the treatment is determined to be effective. Typically however, the tumor stays the same size as it was before treatment (stable disease), or it grows (progressive disease). As used herein the term can apply to any of the treatments or agents described herein, when used as single agents or combinations.

For purposes of the present invention, “co-administration of” and “co-administering” an IGF-1R kinase inhibitor with an additional anti-cancer agent (both components referred to hereinafter as the “two active agents”) refer to any administration of the two active agents, either separately or together, where the two active agents are administered as part of an appropriate dose regimen designed to obtain the benefit of the combination therapy. Thus, the two active agents can be administered either as part of the same pharmaceutical composition or in separate pharmaceutical compositions. The additional agent can be administered prior to, at the same time as, or subsequent to administration of the IGF-1R kinase inhibitor, or in some combination thereof. Where the IGF-1R kinase inhibitor is administered to the patient at repeated intervals, e.g., during a standard course of treatment, the additional agent can be administered prior to, at the same time as, or subsequent to, each administration of the IGF-1R kinase inhibitor, or some combination thereof, or at different intervals in relation to the IGF-1R kinase inhibitor treatment, or in a single dose prior to, at any time during, or subsequent to the course of treatment with the IGF-1R kinase inhibitor.

The IGF-1R kinase inhibitor will typically be administered to the patient in a dose regimen that provides for the most effective treatment of the cancer (from both efficacy and safety perspectives) for which the patient is being treated, as known in the art, and as disclosed, e.g. in International Patent Publication No. WO 01/34574. In conducting the treatment method of the present invention, the IGF-1R kinase inhibitor can be administered in any effective manner known in the art, such as by oral, topical, intravenous, intra-peritoneal, intramuscular, intra-articular, subcutaneous, intranasal, intra-ocular, vaginal, rectal, or intradermal routes, depending upon the type of cancer being treated, the type of IGF-1R kinase inhibitor being used (for example, small molecule, antibody, RNAi, ribozyme or antisense construct), and the medical judgment of the prescribing physician as based, e.g., on the results of published clinical studies.

The amount of IGF-1R kinase inhibitor administered and the timing of IGF-1R kinase inhibitor administration will depend on the type (species, gender, age, weight, etc.) and condition of the patient being treated, the severity of the disease or condition being treated, and on the route of administration. For example, small molecule IGF-1R kinase inhibitors can be administered to a patient in doses ranging from 0.001 to 100 mg/kg of body weight per day or per week in single or divided doses, or by continuous infusion (see for example, International Patent Publication No. WO 01/34574). In particular, compounds such as OSI-906, or similar compounds, can be administered to a patient in doses ranging from 5-200 mg per day, or 100-1600 mg per week, in single or divided doses, or by continuous infusion. Antibody-based IGF-1R kinase inhibitors, or antisense, RNAi or ribozyme constructs, can be administered to a patient in doses ranging from 0.1 to 100 mg/kg of body weight per day or per week in single or divided doses, or by continuous infusion. In some instances, dosage levels below the lower limit of the aforesaid range may be more than adequate, while in other cases still larger doses may be employed without causing any harmful side effect, provided that such larger doses are first divided into several small doses for administration throughout the day.

The IGF-1R kinase inhibitors and other additional agents can be administered either separately or together by the same or different routes, and in a wide variety of different dosage forms. For example, the IGF-1R kinase inhibitor is preferably administered orally or parenterally. Where the IGF-1R kinase inhibitor is OSI-906, or a similar such compound, oral administration is preferable. Both the IGF-1R kinase inhibitor and other additional agents can be administered in single or multiple doses.

The IGF-1R kinase inhibitor can be administered with various pharmaceutically acceptable inert carriers in the form of tablets, capsules, lozenges, troches, hard candies, powders, sprays, creams, salves, suppositories, jellies, gels, pastes, lotions, ointments, elixirs, syrups, and the like. Administration of such dosage forms can be carried out in single or multiple doses. Carriers include solid diluents or fillers, sterile aqueous media and various non-toxic organic solvents, etc. Oral pharmaceutical compositions can be suitably sweetened and/or flavored.

The IGF-1R kinase inhibitor can be combined together with various pharmaceutically acceptable inert carriers in the form of sprays, creams, salves, suppositories, jellies, gels, pastes, lotions, ointments, and the like. Administration of such dosage forms can be carried out in single or multiple doses. Carriers include solid diluents or fillers, sterile aqueous media, and various non-toxic organic solvents, etc.

All formulations comprising proteinaceous IGF-1R kinase inhibitors should be selected so as to avoid denaturation and/or degradation and loss of biological activity of the inhibitor.

Methods of preparing pharmaceutical compositions comprising an IGF-1R kinase inhibitor are known in the art, and are described, e.g. in International Patent Publication No. WO 01/34574. In view of the teaching of the present invention, methods of preparing pharmaceutical compositions comprising an IGF-1R kinase inhibitor will be apparent from the above-cited publications and from other known references, such as Remington's Pharmaceutical Sciences, Mack Publishing Company, Easton, Pa., 18^{th} edition (1990).

For oral administration of IGF-1R kinase inhibitors, tablets containing one or both of the active agents are combined with any of various excipients such as, for example, micro-crystalline cellulose, sodium citrate, calcium carbonate, dicalcium phosphate and glycine, along with various disintegrants such as starch (and preferably corn, potato or tapioca starch), alginic acid and certain complex silicates, together with granulation binders like polyvinyl pyrrolidone, sucrose, gelatin and acacia. Additionally, lubricating agents such as magnesium stearate, sodium lauryl sulfate and talc are often very useful for tableting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the IGF-1R kinase inhibitor may be combined with various sweetening or flavoring agents, coloring matter or dyes, and, if so desired, emulsifying and/or suspending agents as well, together with such diluents as water, ethanol, propylene glycol, glycerin and various like combinations thereof.

For parenteral administration of either or both of the active agents, solutions in either sesame or peanut oil or in aqueous propylene glycol may be employed, as well as sterile aqueous solutions comprising the active agent or a corresponding water-soluble salt thereof. Such sterile aqueous solutions are preferably suitably buffered, and are also preferably rendered isotonic, e.g., with sufficient saline or glucose. These particular aqueous solutions are especially suitable for intravenous, intramuscular, subcutaneous and intraperitoneal injection purposes. The oily solutions are suitable for intra-articular, intramuscular and subcutaneous injection purposes. The preparation of all these solutions under sterile conditions is readily accomplished by standard pharmaceutical techniques well known to those skilled in the art. Any parenteral formulation selected for administration of proteinaceous IGF-1R kinase inhibitors should be selected so as to avoid denaturation and loss of biological activity of the inhibitor.

Additionally, it is possible to topically administer either or both of the active agents, by way of, for example, creams, lotions, jellies, gels, pastes, ointments, salves and the like, in accordance with standard pharmaceutical practice. For example, a topical formulation comprising an IGF-1R kinase inhibitor in about 0.1% (w/v) to about 5% (w/v) concentration can be prepared.

As used herein, the term “IGF-1R kinase inhibitor” refers to any IGF-1R kinase inhibitor that is currently known in the art, and includes any chemical entity that, upon administration to a patient, results in inhibition of a biological activity specifically associated with activation of the IGF-1 receptor (e.g. in humans, the protein encoded by GeneID: 3480) in the patient, and resulting from the binding to IGF-1R of its natural ligand(s). Such IGF-1R kinase inhibitors include any agent that can block IGF-1R activation and the downstream biological effects of IGF-1R activation that are relevant to treating cancer in a patient. Such an inhibitor can act by binding directly to the intracellular domain of the receptor and inhibiting its kinase activity. Alternatively, such an inhibitor can act by occupying the ligand binding site or a portion thereof of the IGF-1 receptor, thereby making the receptor inaccessible to its natural ligand so that its normal biological activity is prevented or reduced. Alternatively, such an inhibitor can act by modulating the dimerization of IGF-1R polypeptides, or interaction of IGF-1R polypeptide with other proteins, or enhance ubiquitination and endocytotic degradation of IGF-1R. An IGF-1R kinase inhibitor can also act by reducing the amount of IGF-1 available to activate IGF-1R, by for example antagonizing the binding of IGF-1 to its receptor, by reducing the level of IGF-1, or by promoting the association of IGF-1 with proteins other than IGF-1R such as IGF binding proteins (e.g. IGFBP3). IGF-1R kinase inhibitors include but are not limited to low molecular weight inhibitors, antibodies or antibody fragments, antisense constructs, small inhibitory RNAs (i.e. RNA interference by dsRNA; RNAi), and ribozymes. In a preferred embodiment, the IGF-1R kinase inhibitor is a small organic molecule or an antibody that binds specifically to the human IGF-1R.

IGF-1R kinase inhibitors include, for example imidazopyrazine IGF-1R kinase inhibitors, quinazoline IGF-1R kinase inhibitors, pyrido-pyrimidine IGF-1R kinase inhibitors, pyrimido-pyrimidine IGF-1R kinase inhibitors, pyrrolo-pyrimidine IGF-1R kinase inhibitors, pyrazolo-pyrimidine IGF-1R kinase inhibitors, phenylamino-pyrimidine IGF-1R kinase inhibitors, oxindole IGF-1R kinase inhibitors, indolocarbazole IGF-1R kinase inhibitors, phthalazine IGF-1R kinase inhibitors, isoflavone IGF-1R kinase inhibitors, quinalone IGF-1R kinase inhibitors, and tyrphostin IGF-1R kinase inhibitors, and all pharmaceutically acceptable salts and solvates of such IGF-1R kinase inhibitors.

Additional examples of IGF-1R kinase inhibitors include those in International Patent Publication No. WO 05/097800, that describes 6,6-bicyclic ring substituted heterobicyclic protein kinase inhibitors, International Patent Publication No. WO 05/037836, that describes imidazopyrazine IGF-1R kinase inhibitors, International Patent Publication Nos. WO 03/018021 and WO 03/018022, that describe pyrimidines for treating IGF-1R related disorders, International Patent Publication Nos. WO 02/102804 and WO 02/102805, that describe cyclolignans and cyclolignans as IGF-1R inhibitors, International Patent Publication No. WO 02/092599, that describes pyrrolopyrimidines for the treatment of a disease which responds to an inhibition of the IGF-1R tyrosine kinase, International Patent Publication No. WO 01/72751, that describes pyrrolopyrimidines as tyrosine kinase inhibitors, and in International Patent Publication No. WO 00/71129, that describes pyrrolotriazine inhibitors of kinases, and in International Patent Publication No. WO 97/28161, that describes pyrrolo[2,3-d]pyrimidines and their use as tyrosine kinase inhibitors, Parrizas, et al., which describes tyrphostins with in vitro and in vivo IGF-1R inhibitory activity (Endocrinology, 138:1427-1433 (1997)), International Patent Publication No. WO 00/35455, that describes heteroaryl-aryl ureas as IGF-1R inhibitors, International Patent Publication No. WO 03/048133, that describes pyrimidine derivatives as modulators of IGF-1R, International Patent Publication No. WO 03/024967, WO 03/035614, WO 03/035615, WO 03/035616, and WO 03/035619, that describe chemical compounds with inhibitory effects towards kinase proteins, International Patent Publication No. WO 03/068265, that describes methods and compositions for treating hyperproliferative conditions, International Patent Publication No. WO 00/17203, that describes pyrrolopyrimidines as protein kinase inhibitors, Japanese Patent Publication No. JP 07/133280, that describes a cephem compound, its production and antimicrobial composition, Albert, A. et al., Journal of the Chemical Society, 11: 1540-1547 (1970), which describes pteridine studies and pteridines unsubstituted in the 4-position, and A. Albert et al., Chem. Biol. Pteridines Proc. Int. Symp., 4th, 4: 1-5 (1969) which describes a synthesis of pteridines (unsubstituted in the 4-position) from pyrazines, via 3-4-dihydropteridines.

IGF-1R kinase inhibitors particularly useful in this invention include compounds represented by Formula (I) (see below), as described in US Published Patent Application US 2006/0235031, where their preparation is described in detail. PQIP (cis-3-[3-(4-Methyl-piperazin-1-yl)-cyclobutyl]1-(2-phenyl-quinolin-7-yl)-imidazo[1,5-a]pyrazin-8-ylamine) and OSI-906 (cis-3-[8-amino-1-(2-phenyl-quinolin-7-yl)-imidazo[1,5-a]pyrazin-3-yl]-1-methyl-cyclobutanol) represents IGF-1R kinase inhibitors according to Formula (I).

OSI-906 has the structure as follows:

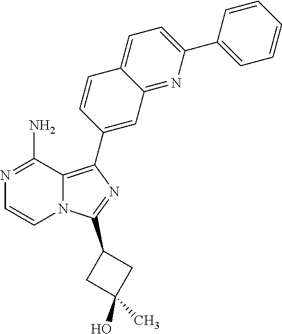

PQIP has the structure as follows:

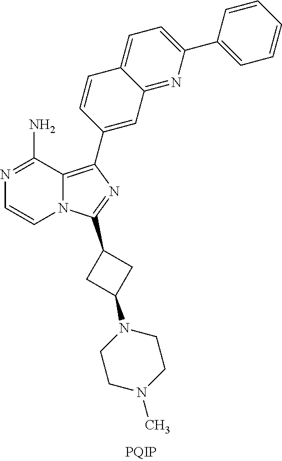

An IGF-1R kinase inhibitor of Formula (I), as described in US Published Patent Application US 2006/0235031, is represented by the formula:

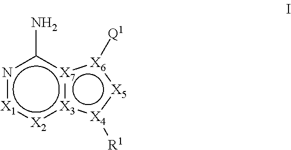

or a pharmaceutically acceptable salt thereof, wherein:

X₁, and X₂ are each independently N or C-(E¹)ₐₐ;

X₅ is N, C-(E¹)ₐₐ, or N-(E¹)ₐₐ;

X₃, X₄, X₆, and X₇ are each independently N or C;

wherein at least one of X₃, X₄, X₅, X₆, and X₇ is independently N or N-(E¹)ₐₐ;

Q¹ is

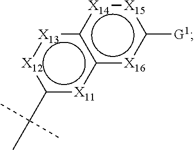

X₁₁, X₁₂, X₁₃, X₁₄, X₁₅, and X₁₆ are each independently N, C-(E¹¹)_{bb}, or N⁺—O⁻;

wherein at least one of X₁₁, X₁₂, X₁₃, X₁₄, X₁₅, and X₁₆ is N or N⁺—O⁻;

R¹ is absent, C₀₋₁₀alkyl, cycloC₃₋₁₀alkyl, bicycloC₅₋₁₀alkyl, aryl, heteroaryl, aralkyl, heteroaralkyl, heterocyclyl, heterobicycloC₅₋₁₀alkyl, spiroalkyl, or heterospiroalkyl, any of which is optionally substituted by one or more independent G¹¹ substituents;

E¹, E¹¹, G¹, and G⁴¹ are each independently halo, —CF₃, —OCF₃, —OR², —NR²R³(R^{2a})ⱼ₁, —C(═O)R², —CO₂R², —CONR²R³, —NO₂, —CN, —S(O)ⱼ₁R², —SO₂NR²R³, —NR²C(═O)R³, —NR²C(═O)OR³, —NR²C(═O)NR³R^{2a}, —NR²S(O)ⱼ₁R³, —C(═S)OR², —C(═O)SR², —NR²C(═NR³)NR^{2a}R^{3a}, —NR²C(═NR³)OR^{2a}, —NR²C(═NR³)SR^{2a}, —OC(═O)OR², —OC(═O)NR²R³, —OC(═O)SR², —SC(═O)OR², —SC(═O)NR²R³, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, oxo, —CF₃, —OCF₃, —OR²²², —NR²²²R³³³(R^{222a})ⱼ₁ₐ, —C(═O)R²²², —CO₂R²²², —C(═O)NR²²²R³³³, —NO₂, —CN, —S(═O)ⱼ₁ₐR²²², —SO₂NR²²²R³³³, —NR²²²C(═O)R³³³, —NR²²²C(═O)OR³³³, —NR²²²C(═O)NR³³³R^{222a}, —NR²²²S(O)ⱼ₁ₐR³³³, —C(═S)OR²²², —C(═O)SR²²², —NR²²²C(═NR³³³)NR^{222a}R^{333a}, —NR²²²C(═NR³³³)OR^{222a}, —NR²²²C(═NR³³³)SR^{222a}, —OC(═O)OR²²², —OC(═O)NR²²²R³³³, —OC(═O)SR²²², —SC(═O)OR²²², or —SC(═O)NR²²²R³³³ substituents;

or E¹, E¹¹, or G¹ optionally is —(W¹)ₙ—(Y¹)ₘ—R⁴;

or E¹, E¹¹, G¹, or G⁴¹ optionally independently is aryl-C₀₋₁₀alkyl, aryl-C₂₋₁₀alkenyl, aryl-C₂₋₁₀alkynyl, hetaryl-C₀₋₁₀alkyl, hetaryl-C₂₋₁₀alkenyl, or hetaryl-C₂₋₁₀alkynyl, any of which is optionally substituted with one or more independent halo, —CF₃, —OCF₃, —OR²²², —NR²²²R³³³(R^{222a})ⱼ₂ₐ, —C(O)R²²², —CO₂R²²², —C(═O)NR²²²R³³³, —NO₂, —CN, —S(O)ⱼ₂ₐR²²², —SO₂NR²²²R³³³, —NR²²²C(═O)R³³³, —NR²²²C(═O)OR³³³, —NR²²²C(═O)NR³³³R^{222a}, —NR²²²S(O)ⱼ₂ₐR³³³, —C(═S)OR²²², —C(═O)SR²²², —NR²²²C(═NR³³³)NR^{222a}R^{333a}, —NR²²²C(═NR³³³)OR^{222a}, —NR²²²C(═NR³³³)SR^{222a}, —OC(═O)OR²²², —OC(═O)NR²²²R³³³, —OC(═O)SR²²², —SC(═O)OR²²², or —SC(═O)NR²²²R³³³ substituents;

G¹¹ is halo, oxo, —CF₃, —OCF₃, —OR²¹, —NR²¹R³¹(R^{2a1})ⱼ₄, —C(O)R²¹, —CO₂R²¹, —C(═O)NR²¹R³¹, —NO₂, —CN, —S(O)ⱼ₄R²¹, —SO₂NR²¹R³¹, NR²¹(C═O)R³¹, NR²¹C(═O)OR³¹, NR²¹C(═O)NR³¹R^{2a1}, NR²¹S(O)ⱼ₄R³¹, —C(═S)OR²¹, —C(═O)SR²¹, —NR²¹C(═NR³¹)NR^{2a1}R^{3a1}, —NR²¹C(═NR³¹)OR^{2a1}, —NR²¹C(═NR³¹)SR^{2a1}, —OC(═O)OR²¹, —OC(═O)NR²¹R³¹, —OC(═O)SR²¹, —SC(═O)OR²¹, —SC(═O)NR²¹R³¹, —P(O)OR²¹OR³¹, C₁₋₁₀alkylidene, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, oxo, —CF₃, —OCF₃, —OR²²²¹, —NR²²²¹R³³³¹(R^{222a1})ⱼ₄ₐ, —C(O)R²²²¹, —CO₂R²²²¹, —C(═O)NR²²²¹R³³³¹, —NO₂, —CN, —S(O)ⱼ₄ₐR²²²¹, —SO₂NR²²²¹R³³³¹, —NR²²²¹C(═O)R³³³¹, —NR²²²¹C(═O)OR³³³¹, —NR²²²¹C(═O)NR³³³¹R^{222a1}, —NR²²²¹S(O)ⱼ₄ₐR³³³¹, —C(═S)OR²²²¹, —C(═O)SR²²²¹, —NR²²²¹C(═NR³³³¹)NR^{222a1}R^{333a1}, —NR²²²¹C(═NR³³³¹)OR^{222a1}, —NR²²²¹C(═NR³³³¹)SR^{222a1}, —OC(═O)OR²²²¹, —OC(═O)NR²²²¹R³³³¹, —OC(═O)SR²²²¹, —SC(═O)OR²²²¹, —P(O)OR²²²¹OR³³³¹, or —SC(═O)NR²²²¹R³³³¹ substituents;

or G¹¹ is aryl-C₀₋₁₀alkyl, aryl-C₂₋₁₀alkenyl, aryl-C₂₋₁₀alkynyl, hetaryl-C₀₋₁₀alkyl, hetaryl-C₂₋₁₀alkenyl, or hetaryl-C₂₋₁₀alkynyl, any of which is optionally substituted with one or more independent halo, —CF₃, —OCF₃, —OR²²²¹, —NR²²²¹R³³³¹(R^{222a1})ⱼ₅ₐ, —C(O)R²²²¹, —CO₂R²²²¹, —C(═O)NR²²²¹R³³³¹, —NO₂, —CN, —S(O)ⱼ₅ₐR²²²¹, —SO₂NR²²²¹R³³³¹, —NR²²²¹C(═O)R³³³¹, —NR²²²¹C(═O)OR³³³¹, —NR²²²¹C(═O)NR³³³¹R^{222a1}, —NR²²²¹S(O)ⱼ₅ₐR³³³¹, —C(═S)OR²²²¹, —C(═O)SR²²²¹, —NR²²²¹C(═NR³³³¹)NR^{222a1}R^{333a1}, —NR²²²¹C(═NR³³³¹)OR^{222a1}, —NR²²²¹C(═NR³³³¹)SR^{222a1}, —OC(═O)OR²²²¹, —OC(═O)NR²²²¹R³³³¹, —OC(═O)SR²²²¹, —SC(═O)OR²²²¹, —P(O)OR²²²¹OR³³³¹, or —SC(═O)NR²²²¹R³³³¹ substituents;

or G¹¹ is C, taken together with the carbon to which it is attached forms a C═C double bond which is substituted with R⁵ and G¹¹¹;

R², R^{2a}, R³, R^{3a}, R²²², R^{222a}, R³³³, R^{333a}, R²¹, R^{2a1}, R³¹, R^{3a1}, R²²²¹, R^{222a1}, R³³³¹, and R^{333a1} are each independently C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, aryl-C₀₋₁₀alkyl, aryl-C₂₋₁₀alkenyl, or aryl-C₂₋₁₀alkynyl, hetaryl-C₀₋₁₀alkyl, hetaryl-C₂₋₁₀alkenyl, or hetaryl-C₂₋₁₀alkynyl, any of which is optionally substituted by one or more independent G¹¹¹ substituents;

or in the case of —NR²R³(R^{2a})ⱼ₁ or —NR²²²R³³³(R^{222a})ⱼ₁ₐ or —NR²²²R³³³(R^{222a})ⱼ₂ₐ or —NR²¹R³¹(R^{2a1})ⱼ₄ or —NR²²²¹R³³³¹(R^{222a1})ⱼ₄ₐ or —NR²²²¹R³³³¹(R^{222a1})ⱼ₅ₐ, then R² and R³, or R²²² and R³³³, or R²²²¹ and R³³³¹, respectfully, are optionally taken together with the nitrogen atom to which they are attached to form a 3-10 membered saturated or unsaturated ring, wherein said ring is optionally substituted by one or more independent G¹¹¹¹ substituents and wherein said ring optionally includes one or more heteroatoms other than the nitrogen to which R² and R³, or R²²² and R³³³, or R²²²¹ and R³³³¹ are attached;

W¹ and Y¹ are each independently —O—, —NR⁷—, —S(O)ⱼ₇—, —CR⁵R⁶—, —N(C(O)OR⁷)—, —N(C(O)R⁷)—, —N(SO₂R⁷)—, —CH₂O—, —CH₂S—, —CH₂N(R⁷)—, —CH(NR⁷)—, —CH₂N(C(O)R⁷)—, —CH₂N(C(O)OR⁷)—, —CH₂N(SO₂R⁷)—, —CH(NHR⁷)—, —CH(NHC(O)R⁷)—, —CH(NHSO₂R⁷)—, —CH(NHC(O)OR⁷)—, —CH(OC(O)R⁷)—, —CH(OC(O)NHR⁷)—, —CH═CH—, —C≡C—, —C(═NOR⁷)—, —C(O)—, —CH(OR⁷)—, —C(O)N(R⁷)—, —N(R⁷)C(O)—, —N(R⁷)S(O)—, —N(R⁷)S(O)₂—, —OC(O)N(R⁷)—, —N(R⁷)C(O)N(R⁸)—, —NR⁷C(O)O—, —S(O)N(R⁷)—, —S(O)₂N(R⁷)—, —N(C(O)R⁷)S(O)—, —N(C(O)R⁷)S(O)₂—, —N(R⁷)S(O)N(R⁸)—, —N(R⁷)S(O)₂N(R⁸)—, —C(O)N(R⁷)C(O)—, —S(O)N(R⁷)C(O)—, —S(O)₂N(R⁷)C(O)—, —OS(O)N(R⁷)—, —OS(O)₂N(R⁷)—, —N(R⁷)S(O)O—, —N(R⁷)S(O)₂O—, —N(R⁷)S(O)C(O)—, —N(R⁷)S(O)₂C(O)—, —SON(C(O)R⁷)—, —SO₂N(C(O)R⁷)—, —N(R⁷)SON(R⁸)—, —N(R⁷)SO₂N(R⁸)—, —C(O)O—, —N(R⁷)P(OR⁸)O—, —N(R⁷)P(OR⁸)—, —N(R⁷)P(O)(OR⁸)O—, —N(R⁷)P(O)(OR⁸)—, —N(C(O)R⁷)P(OR⁸)O—, —N(C(O)R⁷)P(OR⁸)—, —N(C(O)R⁷)P(O)(OR⁸)O—, —N(C(O)R⁷)P(OR⁸)—, —CH(R⁷)S(O)—, —CH(R⁷)S(O)₂—, —CH(R⁷)N(C(O)OR⁸)—, —CH(R⁷)N(C(O)R⁸)—, —CH(R⁷)N(SO₂R⁸)—, —CH(R⁷)O—, —CH(R⁷)S—, —CH(R⁷)N(R⁸)—, —CH(R⁷)N(C(O)R⁸)—, —CH(R⁷)N(C(O)OR⁸)—, —CH(R⁷)N(SO₂R⁸)—, —CH(R⁷)C(═NOR⁸)—, —CH(R⁷)C(O)—, —CH(R⁷)CH(OR⁸)—, —CH(R⁷)C(O)N(R⁸)—, —CH(R⁷)N(R⁸)C(O)—, —CH(R⁷)N(R⁸)S(O)—, —CH(R⁷)N(R⁸)S(O)₂—, —CH(R⁷)OC(O)N(R⁸)—, —CH(R⁷)N(R⁸)C(O)N(R^{7a})—, —CH(R⁷)NR⁸C(O)O—, —CH(R⁷)S(O)N(R⁸)—, —CH(R⁷)S(O)₂N(R⁸)—, —CH(R⁷)N(C(O)R⁸)S(O)—, —CH(R⁷)N(C(O)R⁸)S(O)—, —CH(R⁷)N(R⁸)S(O)N(R^{7a})—, —CH(R⁷)N(R⁸)S(O)₂N(R^{7a})—, —CH(R⁷)C(O)N(R⁸)C(O)—, —CH(R⁷)S(O)N(R⁸)C(O)—, —CH(R⁷)S(O)₂N(R⁸)C(O)—, —CH(R⁷)OS(O)N(R⁸)—, —CH(R⁷)OS(O)₂N(R⁸)—, —CH(R⁷)N(R⁸)S(O)O—, —CH(R⁷)N(R⁸)S(O)₂O—, —CH(R⁷)N(R⁸)S(O)C(O)—, —CH(R⁷)N(R⁸)S(O)₂C(O)—, —CH(R⁷)SON(C(O)R⁸)—, —CH(R⁷)SO₂N(C(O)R⁸)—, —CH(R⁷)N(R⁸)SON(R^{7a})—, —CH(R⁷)N(R⁸)SO₂N(R^{7a})—, —CH(R⁷)C(O)O—, —CH(R⁷)N(R⁸)P(OR^{7a})O—, —CH(R⁷)N(R⁸)P(OR^{7a})—, —CH(R⁷)N(R⁸)P(O)(OR^{7a})O—, —CH(R⁷)N(R⁸)P(O)(OR^{7a})—, —CH(R⁷)N(C(O)R⁸)P(OR^{7a})O—, —CH(R⁷)N(C(O)R⁸)P(OR^{7a})—, —CH(R⁷)N(C(O)R⁸)P(O)(OR^{7a})O—, or —CH(R⁷)N(C(O)R⁸)P(OR^{7a})—;

R⁵, R⁶, G¹¹¹, and G¹¹¹¹ are each independently C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, aryl-C₀₋₁₀alkyl, aryl-C₂₋₁₀alkenyl, aryl-C₂₋₁₀alkynyl, hetaryl-C₀₋₁₀alkyl, hetaryl-C₂₋₁₀alkenyl, or hetaryl-C₂₋₁₀alkynyl, any of which is optionally substituted with one or more independent halo, —CF₃, —OCF₃, —OR⁷⁷, —NR⁷⁷R⁸⁷, —C(O)R⁷⁷, —CO₂R⁷⁷, —CONR⁷⁷R⁸⁷, —NO₂, —CN, —S(O)ⱼ₅ₐR⁷⁷, —SO₂NR⁷⁷R⁸⁷, —NR⁷⁷C(═O)R⁸⁷, —NR⁷⁷C(═O)OR⁸⁷, —NR⁷⁷C(═O)NR⁷⁸R⁸⁷, —NR⁷⁷S(O)ⱼ₅ₐR⁸⁷, —C(═S)OR⁷⁷, —C(═O)SR⁷⁷, —NR⁷⁷C(═NR⁸⁷)NR⁷⁸R⁸⁸, —NR⁷⁷C(═NR⁸⁷)OR⁷⁸, —NR⁷⁷C(═NR⁸⁷)SR⁷⁸, —OC(═O)OR⁷⁷, —OC(═O)NR⁷⁷R⁸⁷, —OC(═O)SR⁷⁷, —SC(═O)OR⁷⁷, —P(O)OR⁷⁷OR⁸⁷, or —SC(═O)NR⁷⁷R⁸⁷ substituents;

or R⁵ with R⁶ are optionally taken together with the carbon atom to which they are attached to form a 3-10 membered saturated or unsaturated ring, wherein said ring is optionally substituted with one or more independent R⁶⁹ substituents and wherein said ring optionally includes one or more heteroatoms;

R⁷, R^{7a}, and R⁸ are each independently acyl, C₀₋₁₀alkyl, C₂₋₁₀alkenyl, aryl, heteroaryl, heterocyclyl or cycloC₃₋₁₀alkyl, any of which is optionally substituted by one or more independent G¹¹¹ substituents;

R⁴ is C₀₋₁₀alkyl, C₂₋₁₀alkenyl, C₂₋₁₀alkynyl, aryl, heteroaryl, cycloC₃₋₁₀alkyl, heterocyclyl, cycloC₃₋₈alkenyl, or heterocycloalkenyl, any of which is optionally substituted by one or more independent G⁴¹ substituents;

R⁶⁹ is halo, —OR⁷⁸, —SH, —NR⁷⁸R⁸⁸, —CO₂R⁷⁸, —C(═O)NR⁷⁸R⁸⁸, —NO₂, —CN, —S(O)ⱼ₈R⁷⁸, —SO₂NR⁷⁸R⁸⁸, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, cyano, nitro, —OR⁷⁷⁸, —SO₂NR⁷⁷⁸R⁸⁸⁸, or —NR⁷⁷⁸R⁸⁸⁸ substituents;

or R⁶⁹ is aryl-C₀₋₁₀alkyl, aryl-C₂₋₁₀alkenyl, aryl-C₂₋₁₀alkynyl, hetaryl-C₀₋₁₀alkyl, hetaryl-C₂₋₁₀alkenyl, hetaryl-C₂₋₁₀alkynyl, mono(C₁₋₆alkyl)aminoC₁₋₆alkyl, di(C₁₋₆alkyl)aminoC₁₋₆alkyl, mono(aryl)aminoC₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —C(═O)NR⁷⁷⁸R⁸⁸⁸, —SO₂NR⁷⁷⁸R⁸⁸⁸, or —NR⁷⁷⁸R⁸⁸⁸ substituents;

or in the case of —NR⁷⁸R⁸⁸, R⁷⁸ and R⁸⁸ are optionally taken together with the nitrogen atom to which they are attached to form a 3-10 membered saturated or unsaturated ring, wherein said ring is optionally substituted with one or more independent halo, cyano, hydroxy, nitro, C₁₋₁₀alkoxy, —SO₂NR⁷⁷⁸R⁸⁸⁸, or —NR⁷⁷⁸R⁸⁸⁸ substituents, and wherein said ring optionally includes one or more heteroatoms other than the nitrogen to which R⁷⁸ and R⁸⁸ are attached;

R⁷⁷, R⁷⁸, R⁸⁷, R⁸⁸, R⁷⁷⁸, and R⁸⁸⁸ are each independently C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀alkylcarbonyl, C₂₋₁₀alkenylcarbonyl, C₂₋₁₀alkynylcarbonyl, C₁₋₁₀alkoxycarbonyl, C₁₋₁₀alkoxycarbonylC₁₋₁₀alkyl, monoC₁₋₆alkylaminocarbonyl, diC₁₋₆alkylaminocarbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂N(C₀₋₄alkyl)(C₀₋₄alkyl), or —N(C₀₋₄alkyl)(C₀₋₄alkyl) substituents;

or R⁷⁷, R⁷⁸, R⁸⁷, R⁸⁸, R⁷⁷⁸, and R⁸⁸⁸ are each independently aryl-C₀₋₁₀alkyl, aryl-C₂₋₁₀alkenyl, aryl-C₂₋₁₀alkynyl, hetaryl-C₀₋₁₀alkyl, hetaryl-C₂₋₁₀alkenyl, hetaryl-C₂₋₁₀alkynyl, mono(C₁₋₆alkyl)aminoC₁₋₆alkyl, di(C₁₋₆alkyl)aminoC₁₋₆alkyl, mono(aryl)aminoC₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —O(C₀₋₄alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄alkyl)(C₀₋₁₀alkyl), —SO₂N(C₀₋₄alkyl)(C₀₋₄alkyl), or —N(C₀₋₄alkyl)(C₀₋₄alkyl) substituents;

n, m, j1, j1a, j2a, j4, j4a, j5a, j7, and j8 are each independently 0, 1, or 2; and aa and bb are each independently 0 or 1.

Additional, specific examples of IGF-1R kinase inhibitors that can be used according to the present invention include h7C10 (Centre de Recherche Pierre Fabre), an IGF-1 antagonist; EM-164 (ImmunoGen Inc.), an IGF-1R modulator; CP-751871 (Pfizer Inc.), an IGF-1 antagonist; lanreotide (Ipsen), an IGF-1 antagonist; IGF-1R oligonucleotides (Lynx Therapeutics Inc.); IGF-1 oligonucleotides (National Cancer Institute); IGF-1R protein-tyrosine kinase inhibitors in development by Novartis (e.g. NVP-AEW541, Garcia-Echeverria, C. et al. (2004) Cancer Cell 5:231-239; or NVP-ADW742, Mitsiades, C. S. et al. (2004) Cancer Cell 5:221-230); IGF-1R protein-tyrosine kinase inhibitors (Ontogen Corp); OSI-906 (OSI Pharmaceuticals); AG-1024 (Camirand, A. et al. (2005) Breast Cancer Research 7:R570-R579 (DOI 10.1186/bcr1028); Camirand, A. and Pollak, M. (2004) Brit. J. Cancer 90:1825-1829; Pfizer Inc.), an IGF-1 antagonist; the tyrphostins AG-538 and I-OMe-AG 538; BMS-536924, a small molecule inhibitor of IGF-1R; PNU-145156E (Pharmacia & Upjohn SpA), an IGF-1 antagonist; BMS 536924, a dual IGF-1R and IR kinase inhibitor (Bristol-Myers Squibb; Huang, F. et al. (2009) Cancer Res. 69(1):161-170); BMS-554417, a dual IGF-1R and IR kinase inhibitor (Bristol-Myers Squibb; Haluska P, et al. Cancer Res 2006; 66(1):362-71); EW541 (Novartis); GSK621659A (Glaxo Smith-Kline); INSM-18 (Insmed); and XL-228 (Exelixis).

Antibody-based IGF-1R kinase inhibitors include any anti-IGF-1R antibody or antibody fragment that can partially or completely block IGF-1R activation by its natural ligand. Antibody-based IGF-1R kinase inhibitors also include any anti-IGF-1 antibody or antibody fragment that can partially or completely block IGF-1R activation. Non-limiting examples of antibody-based IGF-1R kinase inhibitors include those described in Larsson, O. et al (2005) Brit. J. Cancer 92:2097-2101 and Ibrahim, Y. H. and Yee, D. (2005) Clin. Cancer Res. 11:944s-950s, or being developed by Imclone (e.g. A12) or Schering-Plough Research Institute (e.g. 19D12; or as described in US Patent Application Publication Nos. US 2005/0136063 A1 and US 2004/0018191 A1). The IGF-1R kinase inhibitor can be a monoclonal antibody, or an antibody or antibody fragment having the binding specificity thereof. Specific additional anti-IGF-1R antibodies that can be used in the invention include IMCL-A12 (a.k.a. cixutumumab; Imclone), MK-0646 (Merck), CP-751871(a.k.a. figitumumab; Pfizer), AMG-479 (Amgen), and SCH-717454 (a.k.a. robatumumab; Schering-Plough/Merck).

Additional antibody-based IGF-1R kinase inhibitors can be raised according to known methods by administering the appropriate antigen or epitope to a host animal selected, e.g., from pigs, cows, horses, rabbits, goats, sheep, and mice, among others. Various adjuvants known in the art can be used to enhance antibody production.

Although antibodies useful in practicing the invention can be polyclonal, monoclonal antibodies are preferred. Monoclonal antibodies against IGF-1R can be prepared and isolated using any technique that provides for the production of antibody molecules by continuous cell lines in culture. Techniques for production and isolation include but are not limited to the hybridoma technique originally described by Kohler and Milstein (Nature, 1975, 256: 495-497); the human B-cell hybridoma technique (Kosbor et al., 1983, Immunology Today 4:72; Cote et al., 1983, Proc. Nati. Acad. Sci. USA 80: 2026-2030); and the EBV-hybridoma technique (Cole et al, 1985, Monoclonal Antibodies and Cancer Therapy, Alan R. Liss, Inc., pp. 77-96).

Alternatively, techniques described for the production of single chain antibodies (see, e.g., U.S. Pat. No. 4,946,778) can be adapted to produce anti-IGF-1R single chain antibodies. Antibody-based IGF-1R kinase inhibitors useful in practicing the present invention also include anti-IGF-1R antibody fragments including but not limited to F(ab′).sub.2 fragments, which can be generated by pepsin digestion of an intact antibody molecule, and Fab fragments, which can be generated by reducing the disulfide bridges of the F(ab′).sub.2 fragments. Alternatively, Fab and/or scFv expression libraries can be constructed (see, e.g., Huse et al., 1989, Science 246: 1275-1281) to allow rapid identification of fragments having the desired specificity to IGF-1R.

Techniques for the production and isolation of monoclonal antibodies and antibody fragments are well-known in the art, and are described in Harlow and Lane, 1988, Antibodies: A Laboratory Manual, Cold Spring Harbor Laboratory, and in J. W. Goding, 1986, Monoclonal Antibodies: Principles and Practice, Academic Press, London. Humanized anti-IGF-1R antibodies and antibody fragments can also be prepared according to known techniques such as those described in Vaughn, T. J. et al., 1998, Nature Biotech. 16:535-539 and references cited therein, and such antibodies or fragments thereof are also useful in practicing the present invention.

IGF-1R kinase inhibitors for use in the present invention can alternatively be based on antisense oligonucleotide constructs. Anti-sense oligonucleotides, including anti-sense RNA molecules and anti-sense DNA molecules, would act to directly block the translation of IGF-1R mRNA by binding thereto and thus preventing protein translation or increasing mRNA degradation, thus decreasing the level of IGF-1R kinase protein, and thus activity, in a cell. For example, antisense oligonucleotides of at least about 15 bases and complementary to unique regions of the mRNA transcript sequence encoding IGF-1R can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as IGF-1R kinase inhibitors for use in the present invention. IGF-1R gene expression can be reduced by contacting the tumor, subject or cell with a small double stranded RNA (dsRNA), or a vector or construct causing the production of a small double stranded RNA, such that expression of IGF-1R is specifically inhibited (i.e. RNA interference or RNAi). Methods for selecting an appropriate dsRNA or dsRNA-encoding vector are well known in the art for genes whose sequence is known (e.g. see Tuschi, T., et al. (1999) Genes Dev. 13(24):3191-3197; Elbashir, S. M. et al. (2001) Nature 411:494-498; Hannon, G. J. (2002) Nature 418:244-251; McManus, M. T. and Sharp, P. A. (2002) Nature Reviews Genetics 3:737-747; Bremmelkamp, T. R. et al. (2002) Science 296:550-553; U.S. Pat. Nos. 6,573,099 and 6,506,559; and International Patent Publication Nos. WO 01/36646, WO 99/32619, and WO 01/68836).

Ribozymes can also function as IGF-1R kinase inhibitors for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of IGF-1R mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful as IGF-1R kinase inhibitors can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

In the context of the methods of treatment of this invention, IGF-1R kinase inhibitors are used as a composition comprised of a pharmaceutically acceptable carrier and a non-toxic therapeutically effective amount of an IGF-1R kinase inhibitor compound (including pharmaceutically acceptable salts thereof).

The term “pharmaceutically acceptable salts” refers to salts prepared from pharmaceutically acceptable non-toxic bases or acids. When a compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (cupric and cuprous), ferric, ferrous, lithium, magnesium, manganese (manganic and manganous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N′,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When a compound used in the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are citric, hydrobromic, hydrochloric, maleic, phosphoric, sulfuric and tartaric acids.

Pharmaceutical compositions used in the present invention comprising an IGF-1R kinase inhibitor compound (including pharmaceutically acceptable salts thereof) as active ingredient, can include a pharmaceutically acceptable carrier and optionally other therapeutic ingredients or adjuvants. Other therapeutic agents may include those cytotoxic, chemotherapeutic or anti-cancer agents, or agents which enhance the effects of such agents, as listed above. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the IGF-1R kinase inhibitor compounds (including pharmaceutically acceptable salts thereof) of this invention can be combined as the active ingredient in intimate admixture with a pharmaceutical carrier according to conventional pharmaceutical compounding techniques. The carrier may take a wide variety of forms depending on the form of preparation desired for administration, e.g. oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, an IGF-1R kinase inhibitor compound (including pharmaceutically acceptable salts of each component thereof) may also be administered by controlled release means and/or delivery devices. The combination compositions may be prepared by any of the methods of pharmacy. In general, such methods include a step of bringing into association the active ingredients with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

An IGF-1R kinase inhibitor compound (including pharmaceutically acceptable salts thereof) used in this invention, can also be included in pharmaceutical compositions in combination with one or more other therapeutically active compounds. Other therapeutically active compounds may include those cytotoxic, chemotherapeutic or anti-cancer agents, or agents which enhance the effects of such agents, as listed above.

Thus in one embodiment of this invention, the pharmaceutical composition can comprise an IGF-1R kinase inhibitor compound in combination with an anticancer agent, wherein said anti-cancer agent is a member selected from the group consisting of alkylating drugs, antimetabolites, microtubule inhibitors, podophyllotoxins, antibiotics, nitrosoureas, hormone therapies, kinase inhibitors, activators of tumor cell apoptosis, and antiangiogenic agents.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition used for this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably contains from about 0.05 mg to about 5 g of the active ingredient.

For example, a formulation intended for the oral administration to humans may contain from about 0.5 mg to about 5 g of active agent, compounded with an appropriate and convenient amount of carrier material that may vary from about 5 to about 95 percent of the total composition. Unit dosage forms will generally contain between from about 1 mg to about 2 g of the active ingredient, typically 25 mg, 50 mg, 100 mg, 200 mg, 300 mg, 400 mg, 500 mg, 600 mg, 800 mg, or 1000 mg.

Pharmaceutical compositions used in the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions used in the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions for the present invention can be in a form suitable for topical sue such as, for example, an aerosol, cream, ointment, lotion, dusting powder, or the like. Further, the compositions can be in a form suitable for use in transdermal devices. These formulations may be prepared, utilizing an IGF-1R kinase inhibitor compound (including pharmaceutically acceptable salts thereof), via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions for this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing an IGF-1R kinase inhibitor compound (including pharmaceutically acceptable salts thereof) may also be prepared in powder or liquid concentrate form.

Dosage levels for the compounds used for practicing this invention will be approximately as described herein, or as described in the art for these compounds. It is understood, however, that the specific dose level for any particular patient will depend upon a variety of factors including the age, body weight, general health, sex, diet, time of administration, route of administration, rate of excretion, drug combination and the severity of the particular disease undergoing therapy.

The present invention further provides for any of the “methods of treatment” described herein, a corresponding “method for manufacturing a medicament” for use with the same indications and under identical conditions or modalities described for the method of treatment, characterized in that an IGF-1R kinase inhibitor is used, such that where any additional agents, inhibitors or conditions are specified in alternative embodiments of the method of treatment they are also included in the corresponding alternative embodiment for the method for manufacturing a medicament. The present invention also provides an IGF-1R kinase inhibitor for use in any of the methods of treatment for cancer described herein.

Many alternative experimental methods known in the art may be successfully substituted for those specifically described herein in the practice of this invention, as for example described in many of the excellent manuals and textbooks available in the areas of technology relevant to this invention (e.g. Using Antibodies, A Laboratory Manual, edited by Harlow, E. and Lane, D., 1999, Cold Spring Harbor Laboratory Press, (e.g. ISBN 0-87969-544-7); Roe B. A. et. al. 1996, DNA Isolation and Sequencing (Essential Techniques Series), John Wiley & Sons.(e.g. ISBN 0-471-97324-0); Methods in Enzymology: Chimeric Genes and Proteins”, 2000, ed. J. Abelson, M. Simon, S. Emr, J. Thorner. Academic Press; Molecular Cloning: a Laboratory Manual, 2001, 3^{rd} Edition, by Joseph Sambrook and Peter MacCallum, (the former Maniatis Cloning manual) (e.g. ISBN 0-87969-577-3); Current Protocols in Molecular Biology, Ed. Fred M. Ausubel, et. al. John Wiley & Sons (e.g. ISBN 0-471-50338-X); Current Protocols in Protein Science, Ed. John E. Coligan, John Wiley & Sons (e.g. ISBN 0-471-11184-8); and Methods in Enzymology: Guide to protein Purification, 1990, Vol. 182, Ed. Deutscher, M. P., Academic Press, Inc. (e.g. ISBN 0-12-213585-7)), or as described in the many university and commercial websites devoted to describing experimental methods in molecular biology.

This invention will be better understood from the Experimental Details that follow. However, one skilled in the art will readily appreciate that the specific methods and results discussed are merely illustrative of the invention as described more fully in the claims which follow thereafter, and are not to be considered in any way limited thereto.

Experimental Details:

Materials and Methods

IGF-1R Inhibitor Compound

IGF-1R inhibitor compound OSI-906 was provided by OSI Pharmaceuticals, (Melville, N.Y.). OSIP-906 (cis-3-[8-amino-1-(2-phenyl-quinolin-7-yl)-imidazo[1,5-a]pyrazin-3-yl]-1-methyl-cyclobutanol) is synthesized by the methods described in patent application number WO 2005/097800. Compound identity and purity (>99%) were verified by ¹H and ¹³C nuclear magnetic resonance, mass spectrometry (MS), and high-performance liquid chromatography using Bruker Advance 400, WatersMicromass ZQ, and Waters LC Module I Plus instruments, respectively, as well as by elemental analysis. OSI-906 was dissolved in DMSO as a 10 mmol/L stock solution for use in biochemical or cellular assays in vitro.

Cell Lines and Culture. Human cancer cell lines, were obtained from American Type Culture Collection (ATCC, Manassas, Va.), or the following additional indicated sources, and cultured in media as described. Tumor types are also indicated: H295R (adrenocortical carcinoma; ATCC), NCI-H322 (NSCLC; ECACC), NCI-H460 (NSCLC; ATCC), SW1573 (NSCLC; ATCC), H1703 (NSCLC; ATCC), BxPC3 (pancreatic; ATCC), OVCAR5 (ovarian; NCI), MDAH-2774 (ovarian; ATCC), Igrov1 (ovarian; NCI), GEO (colon; Roswell Park Cancer Institute (RPCC)), HT-29 (colon; ATCC), RKO (colon; ATCC), H226 (NSCLC; ATCC), 8226 (myeloma; ATCC), H929 (myeloma; ATCC), U266 (myeloma; ATCC), SKES1 (Ewings sarcoma; ATCC), RDES (Ewings sarcoma; ATCC), RD (rhabdomyosarcoma; ATCC), DU4475 (breast; ATCC), SKNAS (neuroblastoma; ATCC), 2650 (nasal SCC; ATCC), OVCAR4 (ovarian; NCI), A673 (Ewings sarcoma; ATCC), BT474 (breast; ATCC), 1386 (oral SCC; MSKCC, NY), 1186 (SCCHN; MSKCC, NY), Colo205 (colon; ATCC), HCT-15 (colon; ATCC), Fadu (oral SCC; ATCC), SKBR3 (breast; ATCC), 1483 (HNSCC; MSKCC, NY), HSC-2 (HNSCC; RIKEN BioResource Center, Tsukuba, Ibaraki, 305-0074, Japan), SKOV-3 (ovarian; ATCC), OVCAR-3 (ovarian; NCI), OVCAR-8 (ovarian; NCI), CaOV3 (ovarian; ATCC). Cells were maintained at 37° C. in an incubator under an atmosphere containing 5% CO₂. The cells were routinely screened for the presence of mycoplasma (MycoAlert, Cambrex Bio Science, Baltimore, Md.). For growth inhibition assays, cells were plated and allowed to proliferate for 24 hours. After 24 hours, cells had reached approximately 15% confluency, at which time serial dilutions of OSI-906 were added and the cells grown for a further 72 hours. Cell viability was assayed using the Cell Titer-Glo reagent (Promega Corp., Madison, Wis.).

Proliferation Assay. Proliferation was assayed using Cell Titer Glo assays (Promega) and was determined 72 hours following dosing with OSI-906. The basis of the assay is a luminescent quantitation of ATP present in a cell culture plate well. In essence, the greater the number of viable cells in the well, the greater the level of ATP present. The assay utilizes a substrate that binds ATP to produce a luminescent signal, which can be read on a luminometer. Unless otherwise noted, the manufacturer's instructions were followed exactly. Briefly, on Day 1, cells were plated in 120 μl of 10% serum-containing growth media at a density of 4000 cells/well in a white polystyrene 96 well assay plate. On day 2, cells were treated with 15 μl of 10× concentration of the IGF-1R inhibitor (e.g. OSI-906) or DMSO alone for a final well volume of 150 μl. After 72 h incubation with the inhibitor, the cells were assayed. Results were calculated as a fraction of the DMSO controlled cells.

TABLE 1
Mutation Status in Tumor Cell Lines

| Cell Line | KRAS | BRAF | PIK3CA | PTEN |
|---|---|---|---|---|
| GEO | G12A | | | |
| H929 | | | | |
| 8226 | G12A | | | |
| 2650 | | | | |
| H295R | | | nd | |
| MDAH-2774 | G12V | | nd | |
| U266 | | K601N | | |
| H322 | | | | |
| DU4475 | | V600E | | |
| SKES1 | | | | |
| SKNAS | Q61K | | | |
| RDES | nd | nd | nd | |
| OVCAR4 | | | | |
| HT-29 | | V600E | | |
| RD | | | | |
| RKO | | V600E | H1047R | |
| A673 | | V600E | | |
| BT474 | | | K111N | |
| SW1573 | G12C | | K111E | |
| 1386 | nd | nd | Nd | |
| 1186 | nd | nd | nd | |
| OVCAR5 | G12V | | | |
| HCT-15 | G13D | | E545K, D549N | |
| Colo205 | | V600E | | |
| FaDu | | | | |
| Igrov1 | | | | |
| SKBR3 | nd | nd | | |
| 1483 | nd | nd | nd | |
| H460 | Q61H | | E545K | |
| H1703 | | | | |
| BxPC3 | | | | |
| HSC-2 | | | H1047R | |
| MCF7 | | | E545K | |
| T47D | | | H1047R | |
| HCC1954 | | | H1047R | |
| BT20 | | | P539R | |
| A2780 | | | | x |
| EFO-27 | | | | x |
| HSC-4 | | | | x |
| NCI-H446 | | | | x |
| KM12 | | | | G129*, |
| | | | | K267fs*9 |
| MC116 | | | | x |
| BT549 | | | | x |
| HCC70 | | | | x |
| PC3 | | | | R55fs*1 |

nd = not determined;
x = mutation present

Determination of mutant K-RAS status in tumor cells. The KRAS mutation status of tumor cells is that reported by the Sanger Wellcome Trust (See Table 1. Wellcome Trust Genome Campus, Hinxton, Cambridge, CB10 1SA, UK; internet address: www.sanger.ac.uk/genetics/CGP/cosmic/).

For additional tumor cell samples where the KRAS mutation status is unknown, any of the many methods known for determining mutant KRAS status may be employed. For example, for additional tumor cell types, DNA may be isolated using the Qiagen DNA extraction kit (Germantown, Md.). KRAS mutations can be analyzed, for example, by one of the following methods.

Tumor cell samples may be assayed with the DxS Scorpion method (DxS, Manchester, UK) using the manufacturer's instructions. Briefly, template DNA is analyzed for a set of seven known KRAS point mutations in codons 12 and 13 (i.e. G12D (GGT>GAT), G12A (GGT>GCT), G12V (GGT>GTT), G12S (GGT>AGT), G12R (GGT>CGT), G12C (GGT>TGT), and G13D (GGC>GAC)) using the THERASCREEN® KRAS Mutation Detection kit (DxS Ltd., Manchester, UK). Reactions and analysis are performed on a Lightcycler 480 real-time PCR instrument (LC480) that is calibrated using a dye calibration kit provided by the kit manufacturer. Reactions are performed on a 96-well plate in 20 μl reactions using approximately 60 ng of each DNA template. Sample DNA is amplified with eight separate primer sets (one for the wild-type sequence and one for each of seven different point mutations) with an internal Scorpion reporter probe. Cycle cross point (CP) values are calculated using the LC480 Fit-point software suite, and the control CP is subtracted from the CP of each mutation specific primer set. Because there may be spurious low level amplification in the absence of mutant template, amplification products are often visible at later cycle numbers for most of the primer sets. To avoid false-positive results due to background amplification, the assay is considered valid only if the control CP value is less than or equal to 35 cycles. CP thresholds are calculated to compensate for this background amplification. Mutations are called when the CP is less than the statistically-set 5% confidence-value threshold (Franklin W A. et al. (2009) J Mol Diagn: jmoldx.2010.080131v1).

Alternatively, tumor cell samples may be analyzed for KRAS mutations using a high resolution melting temperature method using custom primers and the Roche LC480 real time PCR machine (Mannheim, Germany). Briefly, template DNA is tested by High Resolution Melting (HRM) analysis using a Lightcycler 480 real-time PCR instrument (Roche Applied Science, Indianapolis, Ind.). Approximately 60 ng of tumor template DNA, wild type control DNA and mutant control DNA are amplified on the Lightcycler 480 instrument using HRM master mix (Roche cat #04909631001), with the RASO1 and RASA2 primers and 1.75 mM MgCl₂ in a 10 μl on a 96 well plate, using a 2-step cycling program (95° melting, 72° annealing and extension) for 45 cycles. PCR products are analyzed by HRM with 25 data acquisitions per degree of temperature increase, from 40° to 90° C. Lightcycler 480 Gene Scanning software using the known wild-type control samples for baseline calculation is used for these analyses.

Determination of Mutant B-RAF Status in Tumor Cells.

The B-RAF mutation status of tumor cells is that reported by the Sanger Wellcome Trust (See Table 1; Wellcome Trust Genome Campus, Hinxton, Cambridge, CB10 1SA, UK; internet address—www.sanger.ac.uk/genetics/CGP/cosmic/).

For additional tumor cell samples where the B-RAF mutation status is unknown, any of the many methods known for determining mutant B-RAF status may be employed. For example, for additional tumor cell types, DNA may be isolated using the Qiagen DNA extraction kit (Germantown, Md.). B-RAF mutations can be analyzed, for example, by one of the following methods.

BRAF mutations may be analyzed by PCR amplification and direct sequencing of the products as described previously (Jhawer M, et al. Cancer Res 2008; 68(6):1953-61). For example, suitable primers are F, AACACATTTCAAGCCCCAAA and R, GAAACTGGTTTCAAAATATTCGTT for amplification of exon 15 of BRAF.

Determination of Mutant PIK3CA Status in Tumor Cells.

The PIK3CA mutation status of tumor cells is that reported by the Sanger Wellcome Trust (See Table 1; Wellcome Trust Genome Campus, Hinxton, Cambridge, CB10 1SA, UK; internet address—www.sanger.ac.uk/genetics/CGP/cosmic/). In addition, the PIK3CA mutation status of GEO cells is that reported in Jhawer, M. et al. (2008) Cancer Res. 68(6):1953-1961, and the PIK3CA mutation status of H929 cells is that reported in Muller, C. I. et al. (2007) Leukemia Res. 31:27-32.

For additional tumor cell samples where the PIK3CA mutation status is unknown, any of the many methods known for determining mutant PIK3CA status may be employed. For example, for additional tumor cell types, DNA may be isolated using the Qiagen DNA extraction kit (Germantown, Md.). PIK3CA mutations can be analyzed, for example, by one of the following methods.

PIK3CA mutations may be analyzed by PCR amplification and direct sequencing of the products as described previously (Jhawer M, et al. Cancer Res 2008; 68(6):1953-61). For example, suitable primers for amplification are; F, GCTTTTTCTGTAAATCATCTGTG and R, CTGAGATCAGCCAAATTCAGT for exon 9 of PIK3CA; and F, CATTTGCTCCAAACTGACCA and R, TACTCCAAAGCCTCTTGCTC (for codon 1023 mutation) and F, ACATTCGAAAGACCCTAGCC and R, CAATTCCTATGCAATCGGTCT (for codon 1047 mutation) for exon 20 of PIK3CA.

The PTEN mutation status of tumor cells is that reported by the Sanger Wellcome Trust (See Table 1; Wellcome Trust Genome Campus, Hinxton, Cambridge, CB10 1SA, UK; internet address—www.sanger.ac.uk/genetics/CGP/cosmic/). For additional tumor cell samples where the PTEN mutation status is unknown, any of the many methods known for determining mutant PTEN status may be employed.

Measurement of IGF-1R and IR Phosphorylation. pIGF-1R and pIR were determined by RTK capture array (RTK Proteome Profiler, R&D Systems). Proteome profiler arrays housing 42 different RTKs were purchased from R&D systems (Minneapolis, Minn.) and processed according to the manufacturer's protocol. RTKs included on the array include: HER1, HER2, HER3, HER4, FGFR1, FGFR2a, FGFR3, FGFR4, IR, IGF-1R, Axl, Dtk, Mer, HGFR, MSPR, PDGFRα, PDGFRβ, SCFR, Flt-3, M-CSFR, c-Ret, ROR1, ROR2, Tie-1, Tie-2, TrkA, TrkB, TrkC, VEGFR1, VEGFR2, VEGFR3, MuSK, EphA1, EphA2, EphA3, EphA4, EphA6, EphA7, EphB1, EphB2, EphB4, EphB6. This array was used as an RTK capture assay for determining pIGF-1R and pIR levels.

Determination of IGF2 mRNA levels. The expression of IGF2 mRNA was determined by quantitative PCR. mRNA transcript levels were determined by RT-PCR as follows: Taqman probe and primer sets for IGF2 were obtained from Applied Biosystems (Foster City, Calif.). Quantitation of relative gene expression was conducted as described by the manufacturer using 30 ng of template. In order to determine relative expression across cell lines, amplification of the specific genes was normalized to amplification of the gene for GAPDH. IGF-1 mRNA may be determined by a similar procedure, using IGF1 specific probe and primer sets.

Measurement of apoptosis: Induction of apoptosis as measured by increased Caspase 3/7 activity was determined using the Caspase 3/7 Glo assay (Promega Corporation, Madison, Wis.). Cell lines were seeded at a density of 3000 cells per well in a 96-well plate. 24 hours after plating, cells were dosed with compounds. The signal for Caspase 3/7 Glo was determined 24 hours after dosing. The caspase 3/7 activity was normalized to cell number per well, using a parallel plate treated with Cell Titer Glo (Promega Corporation, Madison, Wis.). Signal for each well was normalized using the following formula: Caspase 3/7 Glo luminescence units/Cell Titer Glo fraction of DMSO control. All graphs were generated using PRISM® software (Graphpad Software, San Diego, Calif.).

Analysis of Additivity and Synergy: The Bliss additivism model was used to classify the effect of combining OSI-906 with paclitaxel as additive, synergistic, or antagonistic. A theoretical curve was calculated for combined inhibition using the equation: E_{bliss}=E_{A}+E_{B}−E_{A}*E_{B}, where E_{A} and E_{B} are the fractional inhibitions obtained by drug A alone and drug B alone at specific concentrations. Here, E_{bliss} is the fractional inhibition that would be expected if the combination of the two drugs was exactly additive. If the experimentally measured fractional inhibition is less than E_{bliss} the combination was said to be synergistic. If the experimentally measured fractional inhibition is greater than E_{bliss} the combination was said to be antagonistic. For dose response curves, the Bliss additivity value was calculated for varying doses of drug A when combined with a constant dose of drug B. This allowed an assessment as to whether drug B affected the potency of drug A or shifted its intrinsic activity. All plots were generated using PRISM® software (Graphpad Software, San Diego, Calif.).

Results

K-RAS Mutations are Predictive of Sensitivity of Ovarian Cancer Cell Growth to IGF-1R Kinase Inhibitors.

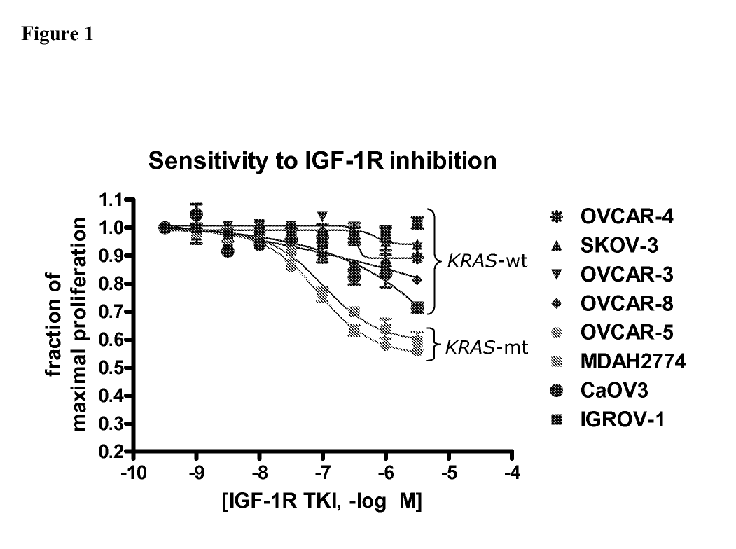

Herein, we find that the dual IGF-1R/IR inhibitor OSI-906 exhibits varying sensitivities to OSI-906 in in vitro proliferation assays for ovarian cancer (OvCa) tumor cell lines. Among a panel of eight tumor cell lines, OVCAR5 and MDAH-2774 cells were sensitive to OSI-906, exhibiting sub-micromolar EC50 values, while the other six cell lines in the panel were relatively insensitive to OSI-906, FIG. 1. OSI-906 sensitivity for the panel correlated with the presence of KRAS activating mutations. Both OVCAR5 and MDAH-2774 cells harbored activating mutations in KRAS, while the other insensitive cell lines harbored WT KRAS. These data suggest that KRAS mutations may be useful to identify OvCa tumors most likely to respond to an IGF-1R inhibitor or an agent that is a dual inhibitor of both IGF-1R and IR. In other tumor cell types tested (e.g. NSCL, CRC, breast), KRAS mutations are found in tumor cells that are sensitive as well as those that are resistant to IGF-1R inhibitors.

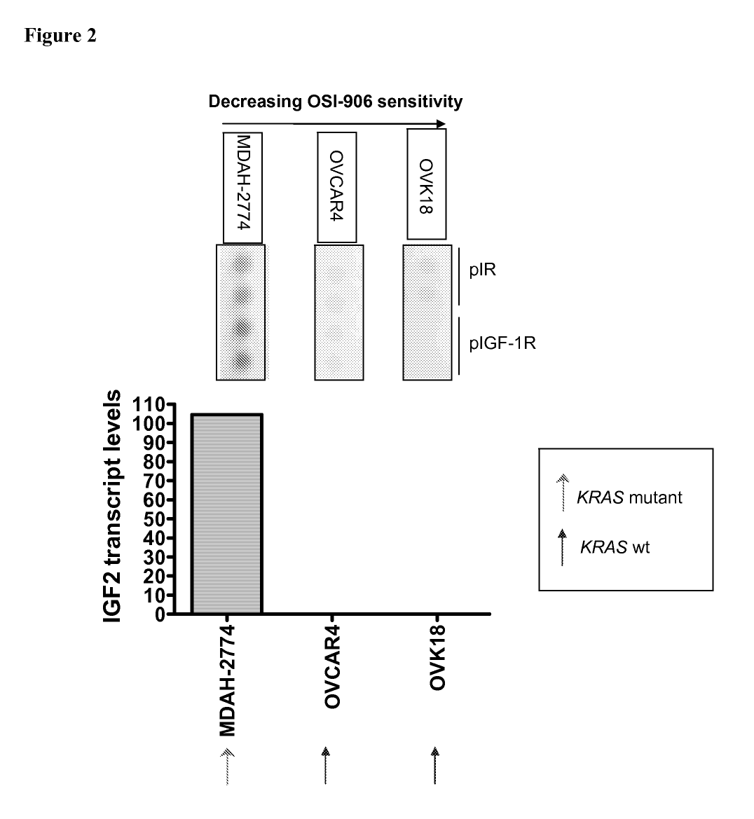

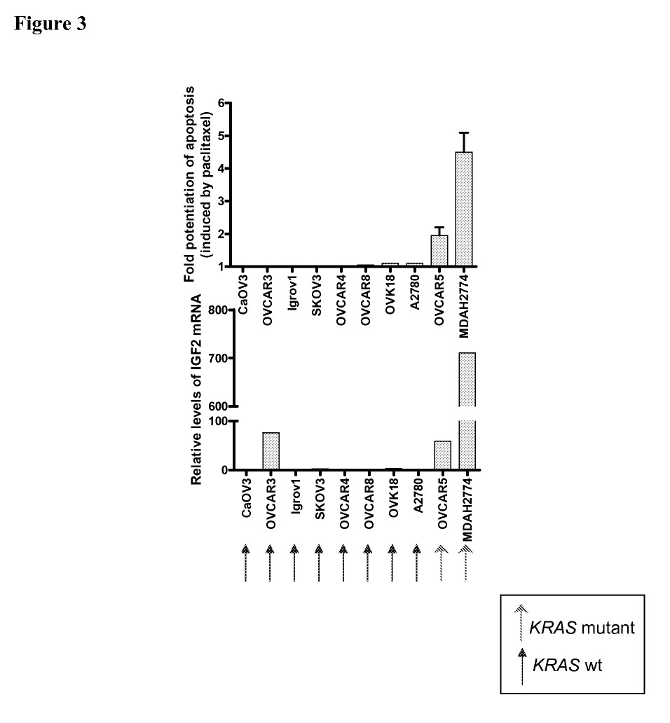

KRAS mutation status and OSI-906 sensitivity also correlated with increased phosphorylation of IGF-1R and IR as well as elevated expression of IGF2 transcripts. The OSI-906 sensitive cell line MDAH-2774 exhibits high expression of IGF2 transcripts as well as a high level of phosphorylation for both IGF-1R and IR, FIG. 2. In contrast two OSI-906 insensitive cell lines, OVK18 and OVCAR4, do not show comparatively high levels of IGF2 transcript expression, and levels of phospho-IGF-1R and IR are below the level of detection. We further find that OSI-906 may enhance the pro-apoptotic effects for paclitaxel in select OvCa tumor cell lines that harbor activating mutations in KRAS and IGF2 autocrine expression, FIG. 3.

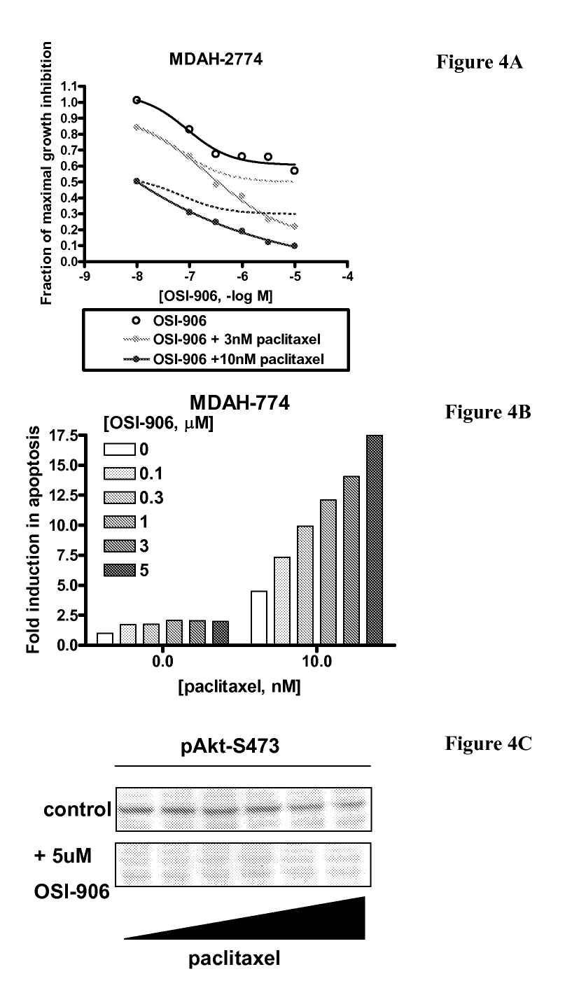

The IGF-1R kinase inhibitor OSI-906 in combination with paclitaxel synergistically inhibits tumor cell growth in ovarian tumor cells that are sensitive to IGF-1R kinase inhubitors (FIG. 4A). This effect is demonstrated at both 3 nM and 10 nM paclitaxel in combination with OSI-906, on MDAH-2774 ovarian tumor cell growth. The dotted line in the plot represents the calculated theoretical result if the combination was additive in nature, and was determined using the Bliss model for additivity. Under these conditions OSI-906 enhances the induction of apoptosis by 10 nM pactitaxel in MDAH-2774 ovarian tumor cells (FIG. 4B). A decrease in the phosphorylation of Akt (i.e. pAKT levels) is observed with 5 μmM OSI-906 in the presence or absence of pactitaxel (100, 30, 10, 3, 1 nM; FIG. 4C).

Characterizing biomarkers predictive of sensitivity to OSI-906+/−chemotherapy would aid our ability to select patient tumors that may optimally benefit from OSI-906. The identification of such biomarkers should have applicability to other IGF-1R/IR inhibitors. In this study we show that there is a correlation between the presence of KRAS mutations and OSI-906 sensitivity. Such a correlation has not been previously established. This finding may provide the foundation for a diagnostic that could be used to identify those OvCa patients most likely to benefit from treatment with OSI-906+/−chemotherapy (e.g. paclitaxel or doxorubicin).

The Presence in Tumor Cells of Either Mutant K-RAS or Mutant B-RAF, in the Absence of Mutant PIK3CA, is Predictive of Sensitivity of Tumor Cell Growth to IGF-1R Kinase Inhibitors.

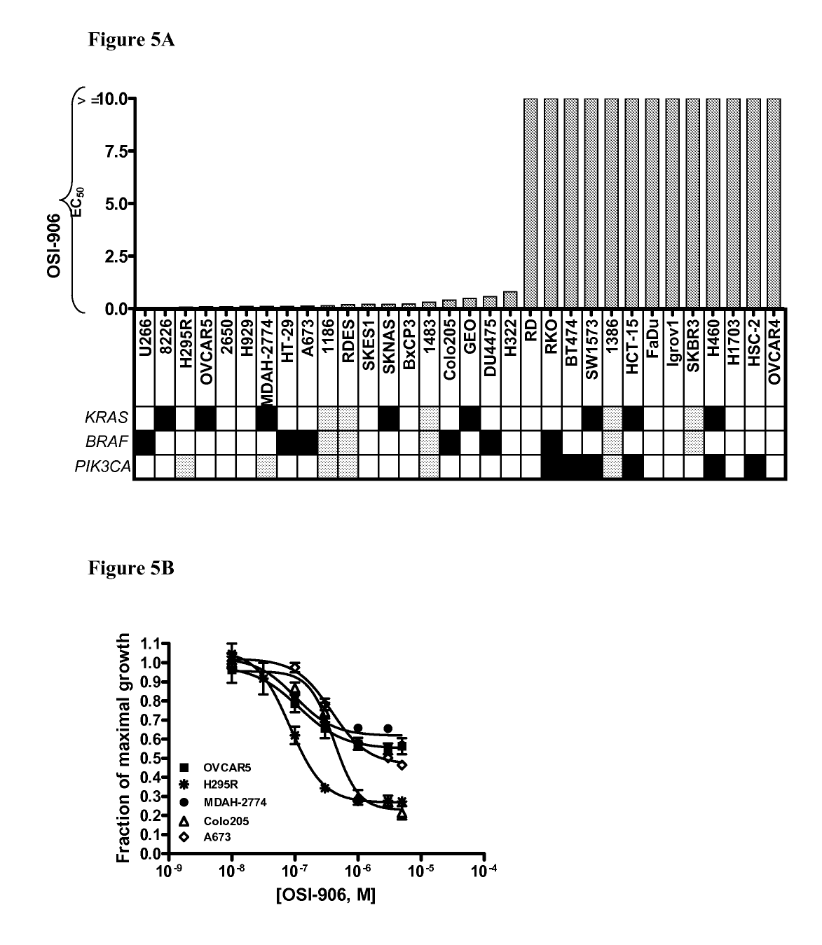

We sought to determine if gene mutations within the IGF-1R/IR axis were predictive of sensitivity to OSI-906, a small molecule dual inhibitor of IGF-1R and IR. OSI-906 selectively inhibits both IGF-1R (IC₅₀=35 nM) and IR (IC₅₀=75 nM) and is far less potent (<50% inhibition at 1 μM) against a broad panel (n=116) of additional RTKs and other protein kinases (Mulvihill M J, et al. Future Medicinal Chemistry 2009; 1(6):1153-71.). A panel of 32 tumor cell lines representing ten tumor types was selected based on differential sensitivity to OSI-906 in cell proliferation assays. Cell lines were categorized as either sensitive (EC₅₀<1 μM) or insensitive (EC₅₀>10 μM) to OSI-906 (FIG. 5A). For sensitive tumor cell lines, growth inhibition by OSI-906 was dose-dependent (FIG. 5B).

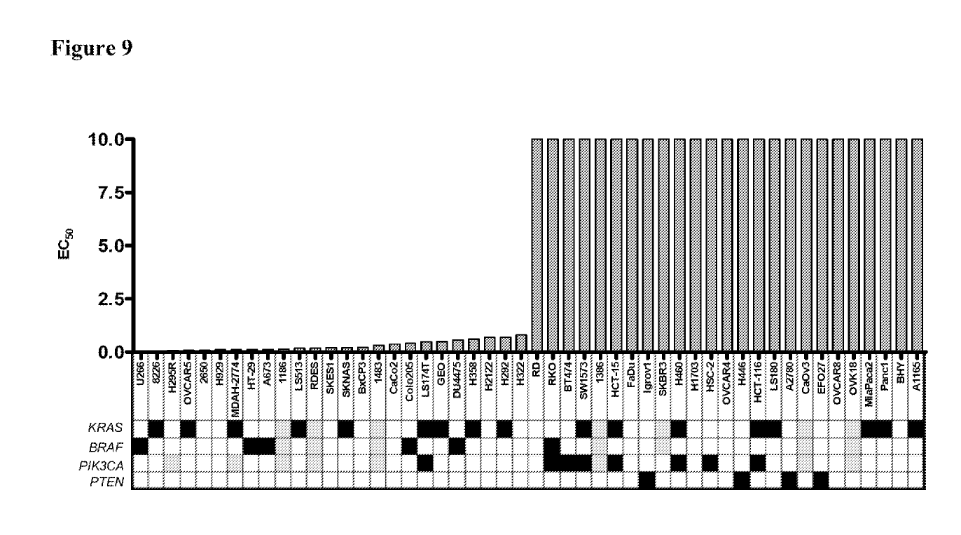

Mutations in KRAS or BRAF are reported to decrease sensitivity to the anti-EGFR antibody cetuximab. However, we found that such mutations occurred frequently in OSI-906-sensitive tumor cell lines. More than two-thirds of the OSI-906-sensitive tumor cells for which the mutational status is known harbor mutations in either KRAS or BRAF, while these mutations were much less frequent (˜27%) in OSI-906-insensitive tumor cells for which the mutational status is known (FIG. 5A). In contrast, mutations in PIK3CA were observed in about half (i.e. ten cell lines, including the breast cancer cell lines T47D, BT20, and HCC1954 not shown in FIG. 9) of the OSI-906-insensitive tumor cell lines for which the mutational status is known, and only occurred in two cell lines that were sensitive to OSI-906 (i.e. the breast cancer cell line MCF7, and the colon cancer cell line LS174T). IGF-1R and IR couple very strongly to the PI3K-AKT pathway, and therefore PIK3CA mutations resulting in constitutive downstream signaling may mitigate the activity of IGF-1R/IR RTK inhibitors.

Analysis of the results indicates that the presence in tumor cells of either mutant K-RAS or mutant B-RAF, in the absence of mutant PIK3CA, correlated with sensitivity of tumor cell growth to the IGF-1R kinase inhibitor. Thus the presence of either K-RAS or B-RAF mutations in tumor cells, in the absence of mutant PIK3CA, is predictive of sensitivity of tumor cell growth to IGF-1R kinase inhibitors, and can be utilized as a diagnostic method to identify patients with cancer who are most likely to benefit from treatment with an IGF-1R kinase inhibitor. Importantly, no tumor types were found with K-RAS or B-RAF mutations, in the absence of mutant PIK3CA, that were insensitive to IGF-1R kinase inhibitors. Tumor types with K-RAS or B-RAF mutations, which had mutant PIK3CA, were insensitive to IGF-1R kinase inhibitors, as were tumor types with no K-RAS or B-RAF mutations, but which had mutant PIK3CA. However, a small number of tumor cells were found to be sensitive to the IGF-1R kinase inhibitor, but did not possess mutant K-RAS or mutant B-RAF. Thus, while a determination of K-RAS, B-RAF and PIK3CA mutation status can be used to identify a large number of tumor cell types that will definitely be sensitive to IGF-1R kinase inhibitors, and also many of those that will be insensitive, absence of K-RAS or B-RAF mutations does not necessarily preclude sensitivity to a IGF-1R kinase inhibitor. All of the above tumor cells that have mutations in either K-RAS or B-RAF, and were found to be sensitive to an IGF-1R kinase inhibitor, were also found to express IGF-1 and/or IGF-1, as judged by mRNA transcript level assessed by RT-PCR, which probably results in autocrine stimulation of tumor cell growth.

In tumor xenograft studies, using tumor cells of a variety of tumor cell types that all have high sensitivity to OSI-906 in culture in vitro (<1 μM EC50), the tumors are also consistently inhibited in vivo with a high percentage tumor growth inhibition (TGI) (e.g. For the following tumor cells, the indicated % TGI was obtained after treatment with OSI-906 in vivo for 10-14 days: H295R: 85%; SKNAS: 71%; BxPC3: 56%; Colo205: 90%). In contast, in similar studies, using tumor cells that have low sensitivity to OSI-906 in culture in vitro (>10 μM EC50), the tumors are inhibited in vivo with only a low pencentage tumor growth inhibition (TGI) (e.g. For the following tumor cells, the indicated % TGI was obtained after treatment with OSI-906 in vivo for 10-14 days: FaDu: <30%; H460: <30%). These data indicate that sensitivity to IGF-1R kinase inhibitors such as OSI-906 in tumor cell culture is predictive of tumor sensitivity in vivo.

Determination if Mutations in Proteins Within Pathways Downstream of IGF-1R/IR Might be Predictive of Sensitivity to OSI-906 Using an Expanded 88 Tumor Cell Line Panel.

To determine if mutations in proteins within pathways downstream of IGF-1R/IR might be predictive of sensitivity to OSI-906 a panel of 88 tumor cell lines was established with varying sensitivity to OSI-906, for which mutations in KRAS, BRAF, PIK3CA, and PTEN had been reported by the Sanger Wellcome Trust. Sensitivity to OSI-906 was determined by measuring the effect of varying concentrations of OSI-906 on cell proliferation following 72 hours of dosing using Cell Titer Glo (Promega). It was found that activating mutations within BRAF trended toward a positive association with OSI-906 sensitivity by Pearson correlation, but this did not reach statistical significance (Table 2). PIK3CA activating mutations trended toward a negative association with OSI-906 sensitivity, however this also did not reach statistical significance. Activating mutations in KRAS were statistically significantly positively associated with OSI-906 sensitivity by Pearson correlation (R=0.22). 39% of OSI-906 sensitive tumor cell lines harbored KRAS mutations, compared with only a rate of 27% in OSI-906 insensitive cell lines. Inactivating mutations in PTEN were statistically significantly negatively associated with OSI-906 sensitivity by Pearson correlation (R=−0.27). All 9 tumor cell lines within the panel which harbored PTEN mutations were insensitive to OSI-906. This included cell lines representing ovarian cancer (A2780 and EFO-27), SCCHN (HSC-4), SCLC (NCI-H446), CRC (KM12), lymphoma (MC116), breast cancer (BT549 and HCC70), and prostate cancer (PC3). Collectively, these data indicate that both KRAS and PTEN mutational status may be a useful determinant of tumor cell OSI-906 sensitivity in the clinic, and may help to identify which patients may benefit from treatment with OSI-906, or other IGF-1R kinase inhibitors.

TABLE 2

| Mutations | n (88) | Correlation | P Value |
|---|---|---|---|
| KRAS | 28 | 0.22 | 0.02 |
| BRAF | 5 | 0.16 | 0.07 |
| PI3K | 12 | −0.11 | 0.16 |
| PTEN | 9 | −0.27 | 0.01 |

Abbreviations

EGF, epidermal growth factor; EMT, epithelial to mesenchymal transition; NSCLC, non-small cell lung carcinoma; SCLC, small cell lung carcinoma; SCC, squamous cell carcinoma; HNSCC or SCCHN, head and neck squamous cell carcinoma; CRC, colorectal cancer; MBC, metastatic breast cancer; EGFR, epidermal growth factor receptor; ErbB3, “v-erb-b2 erythroblastic leukemia viral oncogene homolog 3”, also known as HER-3; pHER3, phosphorylated HER3; Erk kinase, Extracellular signal-regulated protein kinase, also known as mitogen-activated protein kinase; pErk, phosphorylated Erk; Brk, Breast tumor kinase (also known as protein tyrosine kinase 6 (PTK6)); LC, liquid chromatography; MS, mass spectrometry; IGF-1, insulin-like growth factor-1; IGF-2, insulin-like growth factor-2; INSR or IR, insulin receptor; IGF-1R or IGFR, insulin-like growth factor-1 receptor; TGFα, transforming growth factor alpha; HB-EGF, heparin-binding epidermal growth factor; LPA, lysophosphatidic acid; TGFα, transforming growth factor alpha; IC₅₀, half maximal inhibitory concentration; RT, room temperature; pY, phosphotyrosine; pPROTEIN, phospho-PROTEIN, “PROTEIN” can be any protein that can be phosphorylated, e.g. EGFR, ERK, HER3, S6 etc; wt, wild-type; PI3K, phosphatidyl inositol-3 kinase; GAPDH, Glyceraldehyde 3-phosphate dehydrogenase; TKI, Tyrosine Kinase Inhibitor; PMID, PubMed Unique Identifier; NCBI, National Center for Biotechnology Information; NCI, National Cancer Institute; MSKCC, Memorial Sloan Kettering Cancer Center; ECACC, European Collection of Cell Cultures; ATCC, American Type Culture Collection; K-RAS, v-Ki-ras2 Kirsten rat sarcoma viral oncogene homolog; B-RAF, v-raf murine sarcoma viral oncogene homolog B1; PIK3CA, phosphoinositide-3-kinase, catalytic, alpha polypeptide; PTEN, phosphatase and tensin homolog.

### INCORPORATION BY REFERENCE

All patents, published patent applications and other references disclosed herein are hereby expressly incorporated herein by reference.

Equivalents

Those skilled in the art will recognize, or be able to ascertain, using no more than routine experimentation, many equivalents to specific embodiments of the invention described specifically herein. Such equivalents are intended to be encompassed in the scope of the following claims.

1-38. (canceled)

39. A method of identifying patients with ovarian cancer who are most likely to benefit from treatment with an IGF-1R kinase inhibitor, comprising:
determining whether tumor cells from a sample of a patient's tumor possess a mutant K-RAS gene; and
identifying the patient as one most likely to benefit from treatment with an IGF-1R kinase inhibitor if the tumor cells possess a mutant K-RAS gene.

40. The method of claim 39, wherein the IGF-1R kinase inhibitor is a small-molecule IGF-1R kinase inhibitor.

41. The method of claim 40, wherein the small-molecule IGF-1R kinase inhibitor is OSI-906.

42. The method of claim 39, wherein the IGF-1R kinase inhibitor is an anti-IGF-1R antibody or antibody fragment.

43. The method of claim 42, wherein the IGF-1R kinase inhibitor is an anti-IGF-1R antibody selected from the group consisting of cixutumumab, MK-0646, figitumumab, AMG-479, and robatumumab.

44. The method of claim 39, wherein the mutant K-RAS gene is a human K-RAS gene with an activating mutation in codon 12, 13, or 61.

45. A method for treating ovarian cancer in a patient, comprising administering to said patient a therapeutically effective amount of an IGF-1R kinase inhibitor if the patient has been diagnosed to be potentially responsive to an IGF-1R kinase inhibitor by determining that the tumor cells of the patient possess a mutant K-RAS gene.

46. The method of claim 45, wherein the IGF-1R kinase inhibitor is a small-molecule IGF-1R kinase inhibitor.

47. The method of claim 46, wherein the small-molecule IGF-1R kinase inhibitor is OSI-906.

48. The method of claim 45, wherein the IGF-1R kinase inhibitor is an anti-IGF-1R antibody or antibody fragment.

49. The method of claim 48, wherein the IGF-1R kinase inhibitor is an anti-IGF-1R antibody selected from the group consisting of cixutumumab, MK-0646, figitumumab, AMG-479, and robatumumab.

50. The method of claim 45, wherein one or more additional anti-cancer agents are co-administered simultaneously or sequentially with the IGF-1R kinase inhibitor.

51. The method of claim 45, wherein the mutant K-RAS gene is a human K-RAS gene with an activating mutation in codon 12, 13, or 61.

52. A method of identifying patients with cancer who are most likely to benefit from treatment with an IGF-1R kinase inhibitor, comprising:
determining if tumor cells from a sample of a patient's tumor possess a mutant K-RAS gene or a mutant B-RAF gene;
determining if tumor cells of the sample possess a mutant PIK3CA gene; and
identifying the patient as likely to benefit from treatment with an IGF-1R kinase inhibitor if mutant K-ras or mutant B-RAF is present in the tumor cells of the patient in the absence of mutant PIK3CA.

53. The method of claim 52, wherein the IGF-1R kinase inhibitor is a small-molecule IGF-1R kinase inhibitor.

54. The method of claim 53, wherein the small-molecule IGF-1R kinase inhibitor is OSI-906.

55. The method of claim 52, wherein the IGF-1R kinase inhibitor is an anti-IGF-1R antibody or antibody fragment.

56. The method of claim 55, wherein the IGF-1R kinase inhibitor is an anti-IGF-1R antibody selected from the group consisting of cixutumumab, MK-0646, figitumumab, AMG-479, and robatumumab.

57. The method of claim 52, wherein the mutant K-RAS gene is a human K-RAS gene with an activating mutation in codon 12, 13, or 61.

58. The method of claim 52, wherein the mutant B-RAF gene is a human B-RAF gene with an activating mutation in codon 600 or 601.

59. The method of claim 52, wherein the mutant PIK3CA gene is a human PIK3CA gene with an activating mutation in codon 111, 542, 545, 549, or 1047.

60. The method of claim 52, wherein the tumor cells are from a cancer selected from myeloma, NSCLC, ACC, ovarian cancer, HNSCC, colon cancer, Ewing's sarcoma, rhabdomyosarcoma, neuroblastoma, pancreatic cancer, or breast cancer.

61. A method for treating cancer in a patient, comprising administering to said patient a therapeutically effective amount of an IGF-1R kinase inhibitor if the patient has been diagnosed to be potentially responsive to an IGF-1R kinase inhibitor by determining that the tumor cells of the patient possess a mutant K-RAS or mutant B-RAF gene in the absence of a mutant PIK3CA gene.

62. The method of claim 61, wherein the IGF-1R kinase inhibitor is a small-molecule IGF-1R kinase inhibitor.

63. The method of claim 62, wherein the small-molecule IGF-1R kinase inhibitor is OSI-906.

64. The method of claim 61, wherein the IGF-1R kinase inhibitor is an anti-IGF-1R antibody or antibody fragment.

65. The method of claim 64, wherein the IGF-1R kinase inhibitor is an anti-IGF-1R antibody selected from the group consisting of cixutumumab, MK-0646, figitumumab, AMG-479, and robatumumab.

66. The method of claim 61, wherein one or more additional anti-cancer agents are co-administered simultaneously or sequentially with the IGF-1R kinase inhibitor.

67. The method of claim 61, wherein the mutant K-RAS gene is a human K-RAS gene with an activating mutation in codon 12, 13, or 61.

68. The method of claim 61, wherein the mutant B-RAF gene is a human B-RAF gene with an activating mutation in codon 600 or 601.

69. The method of claim 61, wherein the mutant PIK3CA gene is a human PIK3CA gene with an activating mutation in codon 111, 542, 545, 549, or 1047.

70. The method of claim 61, wherein the tumor cells are from a cancer selected from myeloma, NSCLC, ACC, ovarian cancer, HNSCC, colon cancer, Ewing's sarcoma, rhabdomyosarcoma, neuroblastoma, pancreatic cancer, or breast cancer.

71. A method of predicting the sensitivity of tumor cell growth to inhibition by an IGF-1R kinase inhibitor in a patient, comprising: determining if tumor cells from a sample of a patient's tumor possess a mutant PTEN gene or a mutant PIK3CA gene; and concluding that if the tumor cells possess mutant PTEN or mutant PIK3CA, low sensitivity to growth inhibition by an IGF-1R kinase inhibitor is predicted in the patient, based upon a predetermined correlation of the presence of mutant PTEN or mutant PIK3CA with low sensitivity.

72. The method of claim 71, wherein the IGF-1R kinase inhibitor is a small-molecule IGF-1R kinase inhibitor.

73. The method of claim 72, wherein the small-molecule IGF-1R kinase inhibitor is OSI-906.

74. The method of claim 71, wherein the IGF-1R kinase inhibitor is an anti-IGF-1R antibody or antibody fragment.

75. The method of claim 74, wherein the IGF-1R kinase inhibitor is an anti-IGF-1R antibody selected from the group consisting of cixutumumab, MK-0646, figitumumab, AMG-479, and robatumumab.

76. The method of claim 74, wherein the mutant PIK3CA gene is a human PIK3CA gene with an activating mutation in codon 111, 542, 545, 549, or 1047.

77. The method of claim 74, wherein the tumor cells are from a cancer selected from myeloma, NSCLC, ACC, ovarian cancer, HNSCC, colon cancer, Ewing's sarcoma, rhabdomyosarcoma, neuroblastoma, pancreatic cancer, or breast cancer.

78. A method for treating cancer in a patient, comprising administering to said patient a therapeutically effective amount of an IGF-1R kinase inhibitor if the patient has been diagnosed to be potentially responsive to an IGF-1R kinase inhibitor by a determination that the tumor cells of the patient do not possess a mutant PTEN gene or a mutant PIK3CA gene.

79. The method of claim 78, wherein the IGF-1R kinase inhibitor is a small-molecule IGF-1R kinase inhibitor.

80. The method of claim 79, wherein the small-molecule IGF-1R kinase inhibitor is OSI-906.

81. The method of claim 78, wherein the IGF-1R kinase inhibitor is an anti-IGF-1R antibody or antibody fragment.

82. The method of claim 81, wherein the IGF-1R kinase inhibitor is an anti-IGF-1R antibody selected from the group consisting of cixutumumab, MK-0646, figitumumab, AMG-479, and robatumumab.

83. The method of claim 78, wherein one or more additional anti-cancer agents are co-administered simultaneously or sequentially with the IGF-1R kinase inhibitor.

84. The method of claim 78, wherein the mutant PIK3CA gene is a human PIK3CA gene with an activating mutation in codon 111, 542, 545, 549, or 1047.

85. The method of claim 78, wherein the tumor cells are from a cancer selected from myeloma, NSCLC, ACC, ovarian cancer, HNSCC, colon cancer, Ewing's sarcoma, rhabdomyosarcoma, neuroblastoma, pancreatic cancer, or breast cancer.
